(19) United States
(12) Patent Application Publication
(10) Pub. No.: US 2024/0138259 A1
(43) Pub. Date: Apr. 25, 2024

(54) ORGANIC COMPOUND, LIGHT-EMITTING DEVICE, LIGHT-EMITTING APPARATUS, ELECTRONIC DEVICE, AND LIGHTING DEVICE

(71) Applicant: SEMICONDUCTOR ENERGY LABORATORY CO., LTD., Atsugi-shi, Kanagawa-ken (JP)

(72) Inventors: Yui YOSHIYASU, Atsugi, Kanagawa (JP); Hideko YOSHIZUMI, Atsugi, Kanagawa (JP); Nobuharu OHSAWA, Zama, Kanagawa (JP); Satoshi SEO, Sagamihara, Kanagawa (JP)

(21) Appl. No.: 18/262,154

(22) PCT Filed: Jan. 12, 2022
(86) PCT No.: PCT/IB2022/050197

§ 371 Date: Jul. 19, 2023

(30) Foreign Application Priority Data
Jan. 22, 2021 (JP) 2021-008653

(51) Int. Cl. H10K 85/60; C07D 491/22; C09K 11/06

(52) U.S. Cl. CPC H10K 85/6572; C07D 491/22; C09K 11/06; H10K 85/636; H10K 85/657; H10K 50/11

(57) ABSTRACT

A novel organic compound is to be provided. In other words, a novel organic compound effective in improving element characteristics is to be provided. The organic compound is represented by General Formula (G1) shown below.

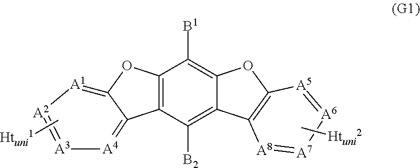

In General Formula (G1) above, at least one or two of A¹ to A⁴ represent nitrogen, and the others represent carbon. At least one or two of A⁵ to A⁸ represent nitrogen, and the others represent carbon. In addition, B¹ and B² each independently represent hydrogen, an alkyl group having 1 to 6 carbon atoms, or a cyano group. In addition, Htᵤₙᵢ¹ and Htᵤₙᵢ² each independently represent a hole-transport skeleton.

### TECHNICAL FIELD

One embodiment of the present invention relates to an organic compound, a light-emitting device, a light-emitting apparatus, an electronic device, and a lighting device. Note that one embodiment of the present invention is not limited to the above technical field. That is, one embodiment of the present invention relates to an object, a method, a manufacturing method, or a driving method. Another embodiment of the present invention relates to a process, a machine, manufacture, or a composition of matter. Specific examples include a semiconductor device, a display device, and a liquid crystal display device.

### Background Art

Light-emitting devices (organic EL devices) including organic compounds and utilizing electroluminescence (EL) have been put to more practical use. As a basic structure of such a light-emitting device, an organic compound layer containing a light-emitting substance (EL layer) is sandwiched between a pair of electrodes. When a voltage is applied to the light-emitting device, electrons and holes injected from the electrodes are recombined, which brings the light-emitting substance (organic compound) contained in the EL layer into an excited state; and light is emitted when the light-emitting substance returns to the ground state from the excited state.

The excited state can be a singlet excited state (S*) and a triplet excited state (T*): light emission from a singlet excited state is referred to as fluorescence, and light emission from a triplet excited state is referred to as phosphorescence. The statistical generation ratio thereof in the light-emitting device is considered to be S*:T*=1:3. Since the emission spectrum obtained from a light-emitting substance depends on the light-emitting substance, the use of different types of organic compounds as light-emitting substances offers light-emitting devices exhibiting various emission colors.

In order to improve device characteristics of such a light-emitting device, improvement of a device structure, development of a material, and the like have been actively carried out (see Patent Document 1, for example).

### REFERENCE

### Patent Document

[Patent Document 1] Japanese Published Patent Application No. 2010-182699

### SUMMARY OF THE INVENTION

### Problems to be Solved by the Invention

In development of light-emitting devices, organic compounds used in the light-emitting devices are very important for improving the characteristics. Thus, in one embodiment of the present invention, a novel organic compound is provided. That is, a novel organic compound that is effective in improving the element characteristics is provided. In one embodiment of the present invention, a novel organic compound that can be used in a light-emitting device is provided. In one embodiment of the present invention, a novel organic compound that can be used in an EL layer of a light-emitting device is provided. In addition, a highly efficient light-emitting device using a novel organic compound of one embodiment of the present invention is provided. In addition, a light-emitting device using a novel organic compound of one embodiment of the present invention and emitting blue light with high color purity is provided. In addition, a novel light-emitting apparatus, a novel electronic device, or a novel lighting device is provided.

The description of these objects does not preclude the existence of other objects. In one embodiment of the present invention, there is no need to achieve all of these objects. Other objects will be apparent from the description of the specification, the drawings, the claims, and the like, and other objects can be derived from the description of the specification, the drawings, the claims, and the like.

### Means for Solving the Problems

One embodiment of the present invention is an organic compound represented by General Formula (G1) below.

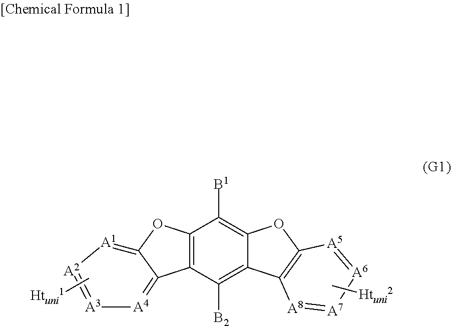

In General Formula (G1) shown above, at least one or two of A¹ to A⁴ represent nitrogen, and the others represent carbon. At least one or two of A⁵ to A⁸ represent nitrogen, and the others represent carbon. In addition, B¹ and B² each independently represent hydrogen, an alkyl group having 1 to 6 carbon atoms, or a cyano group. In addition, Htᵤₙᵢ¹ and Htᵤₙᵢ² each independently represent a hole-transport skeleton. Note that carbon in General Formula (G1) shown above may be bonded to hydrogen or a substituent.

In the above structure, it is preferable that Htᵤₙᵢ¹ and Htᵤₙᵢ² each independently have a carbazolyl group or an amino group.

In the above structure, it is preferable that Htᵤₙᵢ¹ and the Htᵤₙᵢ² be each independently a substituent represented by General Formula (Ht-1) or (Ht-2) below.

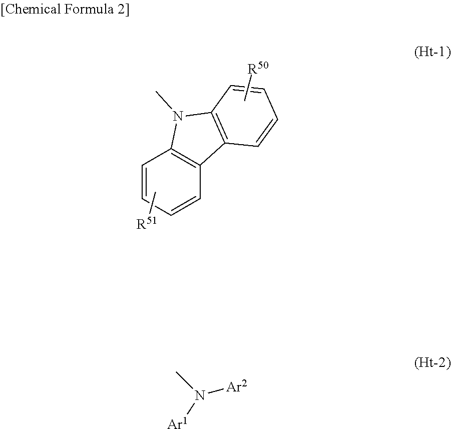

In General Formula (Ht-1) or (Ht-2) shown above, R⁵⁰ and R⁵¹ each represent 1 to 4 substituents and independently represent any one of hydrogen, an alkyl group having 1 to 6 carbon atoms, and a substituted or unsubstituted phenyl group. In addition, Ar¹ and Ar² represent any one of a substituted or unsubstituted phenyl group, a substituted or unsubstituted naphthyl group, a substituted or unsubstituted carbazolyl group, a substituted or unsubstituted fluorenyl group, a substituted or unsubstituted dibenzofuranyl group, and a substituted or unsubstituted dibenzothiophenyl group.

Another embodiment of the present invention is an organic compound represented by General Formula (G2) below.

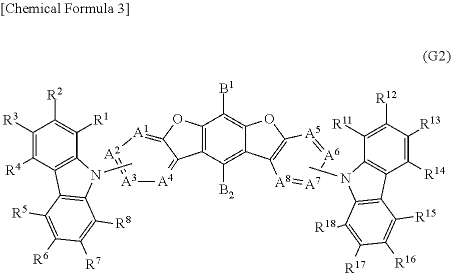

In General Formula (G2) shown above, at least one or two of A¹ to A⁴ represent nitrogen, and the others represent carbon. At least one or two of A⁵ to A⁸ represent nitrogen, and the others represent carbon. In addition, B¹ and B² each independently represent hydrogen, an alkyl group having 1 to 6 carbon atoms, or a cyano group. Furthermore, R¹ to R⁸ and R¹¹ to R¹⁸ each independently represent hydrogen, an alkyl group having 1 to 6 carbon atoms, a substituted or unsubstituted monocyclic saturated hydrocarbon group having 5 to 7 carbon atoms, a substituted or unsubstituted polycyclic saturated hydrocarbon group having 7 to 10 carbon atoms, a substituted or unsubstituted aryl group having 6 to 13 carbon atoms, or a substituted or unsubstituted heteroaryl group having 3 to 12 carbon atoms.

Another embodiment of the present invention is an organic compound represented by General Formula (G3) below.

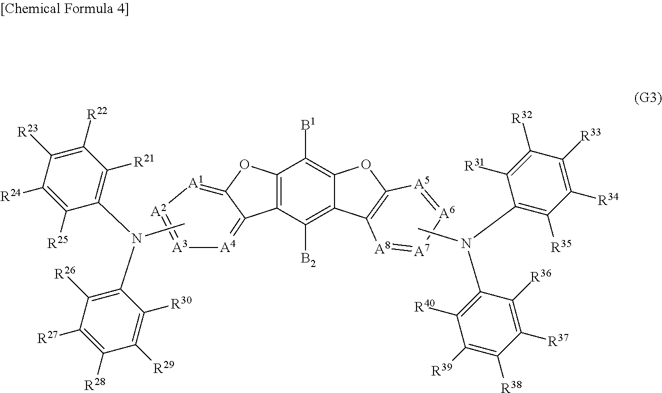

In General Formula (G3) shown above, at least one or two of A¹ to A⁴ represent nitrogen, and the others represent carbon. In addition, at least one or two of A⁵ to A⁸ represent nitrogen, and the others represent carbon. In addition, B¹ and B² each independently represent hydrogen, an alkyl group having 1 to 6 carbon atoms, or a cyano group. Furthermore, R²¹ to R³⁰ and R³¹ to R⁴⁰ each independently represent hydrogen, an alkyl group having 1 to 6 carbon atoms, a substituted or unsubstituted monocyclic saturated hydrocarbon group having 5 to 7 carbon atoms, a substituted or unsubstituted polycyclic saturated hydrocarbon group having 7 to 10 carbon atoms, a substituted or unsubstituted aryl group having 6 to 13 carbon atoms, or a substituted or unsubstituted heteroaryl group having 3 to 12 carbon atoms.

Another embodiment of the present invention is an organic compound represented by General Formula (G4) below.

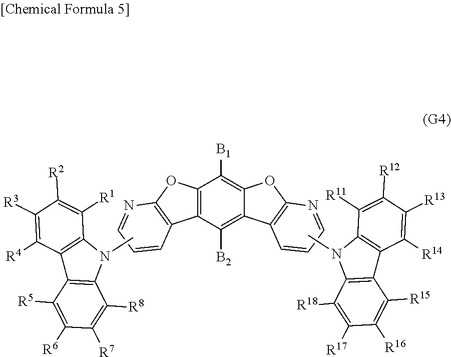

In General Formula (G4) shown above, B¹ and B² each independently represent hydrogen, an alkyl group having 1 to 6 carbon atoms, or a cyano group. Furthermore, R¹ to R⁸ and R¹¹ to R¹⁸ each independently represent hydrogen, an alkyl group having 1 to 6 carbon atoms, a substituted or unsubstituted monocyclic saturated hydrocarbon group having 5 to 7 carbon atoms, a substituted or unsubstituted polycyclic saturated hydrocarbon group having 7 to 10 carbon atoms, a substituted or unsubstituted aryl group having 6 to 13 carbon atoms, or a substituted or unsubstituted heteroaryl group having 3 to 12 carbon atoms.

Another embodiment of the present invention is an organic compound represented by General Formula (G5) below.

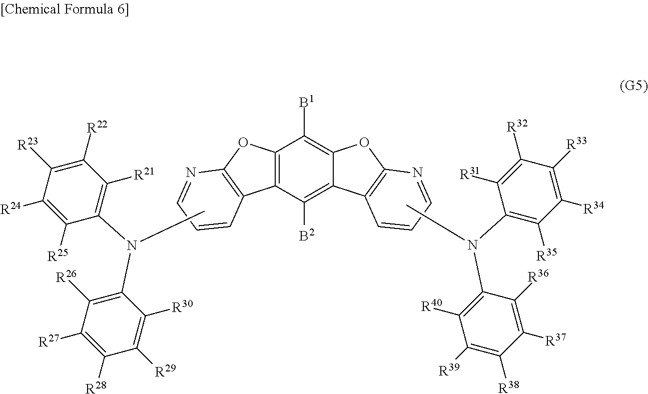

In General Formula (G5) shown above, B¹ and B² each independently represent hydrogen, an alkyl group having 1 to 6 carbon atoms, or a cyano group. Furthermore, R²¹ to R³⁰ and R³¹ to R⁴⁰ each independently represent hydrogen, an alkyl group having 1 to 6 carbon atoms, a substituted or unsubstituted monocyclic saturated hydrocarbon group having 5 to 7 carbon atoms, a substituted or unsubstituted polycyclic saturated hydrocarbon group having 7 to 10 carbon atoms, a substituted or unsubstituted aryl group having 6 to 13 carbon atoms, or a substituted or unsubstituted heteroaryl group having 3 to 12 carbon atoms.

Another embodiment of the present invention is an organic compound represented by Structural Formula (100), (101), or (102) below.

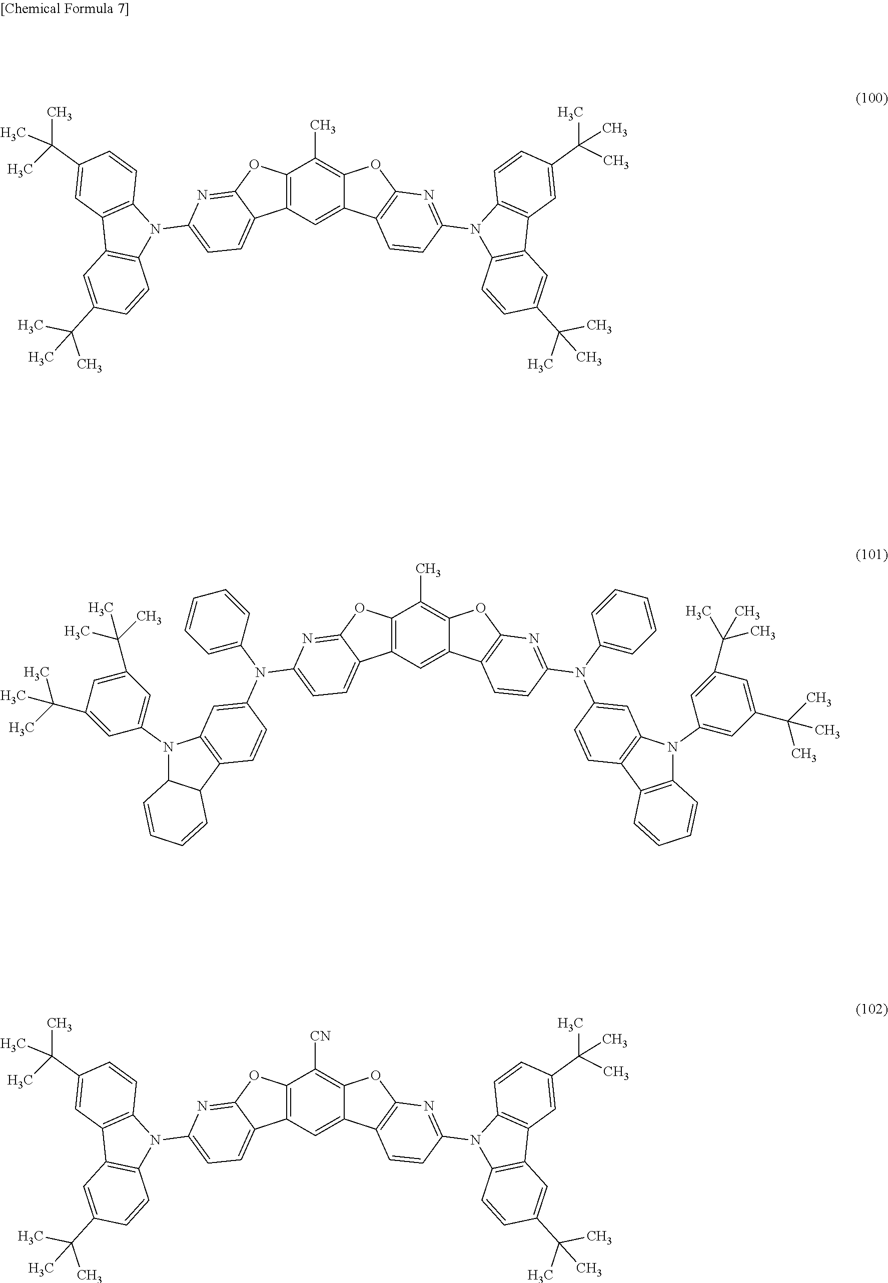

Another embodiment of the present invention is a light-emitting device using the above-described organic compound of one embodiment of the present invention.

Another embodiment of the present invention is a light-emitting device using the above-described organic compound of one embodiment of the present invention. Note that the present invention also includes a light-emitting device that is formed using the organic compound of one embodiment of the present invention for an EL layer between a pair of electrodes or a light-emitting layer included in the EL layer. In addition to the light-emitting devices, a light-emitting apparatus including a transistor, a substrate, and the like is also included in the scope of the invention. Furthermore, in addition to the light-emitting apparatus, an electronic device and a lighting device that include a microphone, a camera, an operation button, an external connection portion, a housing, a cover, a support, a speaker, or the like are also included in the scope of the invention.

In addition, the scope of one embodiment of the present invention includes a light-emitting apparatus including a light-emitting device, and a lighting device including the light-emitting apparatus. Accordingly, a light-emitting apparatus in this specification refers to an image display device or a light source (including a lighting device). In addition, a light-emitting apparatus includes a module in which a light-emitting apparatus is connected to a connector such as an FPC (Flexible Printed Circuit) or a TCP (Tape Carrier Package), a module in which a printed wiring board is provided on the tip of a TCP, or a module in which an IC (integrated circuit) is directly mounted on a light-emitting device by a COG (Chip On Glass) method.

### Effect of the Invention

According to one embodiment of the present invention, a novel organic compound can be provided. That is, a novel organic compound that is effective in improving the element characteristics can be provided. In one embodiment of the present invention, a novel organic compound that can be used in a light-emitting device can be provided. In one embodiment of the present invention, a novel organic compound that can be used in an EL layer of a light-emitting device can be provided. In addition, a highly efficient light-emitting device using a novel organic compound of one embodiment of the present invention can be provided. In addition, a highly efficient light-emitting device can be provided by using a novel organic compound of one embodiment of the present invention. In addition, a novel light-emitting apparatus, a novel electronic device, or a novel lighting device can be provided.

Note that the description of these effects does not preclude the existence of other effects. One embodiment of the present invention does not need to have all the effects. Other effects will be apparent from the description of the specification, the drawings, the claims, and the like, and other effects can be derived from the description of the specification, the drawings, the claims, and the like.

### BRIEF DESCRIPTION OF THE DRAWINGS

FIG. 1A to FIG. 1E are diagrams illustrating a structure of a light-emitting device of one embodiment.

FIG. 2A and FIG. 2B are diagrams illustrating structures of a light-emitting apparatus of one embodiment.

FIG. 3A and FIG. 3B are diagrams illustrating a manufacturing method of a light-emitting apparatus of one embodiment.

FIG. 4A to FIG. 4C are diagrams illustrating a manufacturing method of a light-emitting apparatus of one embodiment.

FIG. 5A to FIG. 5C are diagrams illustrating a manufacturing method of a light-emitting apparatus of one embodiment.

FIG. 6A and FIG. 6B are diagrams illustrating a manufacturing method of a light-emitting apparatus of one embodiment.

FIG. 7 is a diagram illustrating a structure of a light-emitting apparatus of one embodiment.

FIG. 8A and FIG. 8B are diagrams illustrating a light-emitting apparatus of one embodiment.

FIG. 9 is a diagram illustrating a manufacturing method of a light-emitting apparatus of one embodiment.

FIG. 10A to FIG. 10C are diagrams illustrating a manufacturing method of a light-emitting apparatus of one embodiment.

FIG. 11A and FIG. 11B are diagrams illustrating a manufacturing method of a light-emitting apparatus of one embodiment.

FIG. 12 is a diagram illustrating a light-emitting apparatus of one embodiment.

FIG. 13A and FIG. 13B are diagrams illustrating a light-emitting apparatus of one embodiment.

FIG. 14A and FIG. 14B are diagrams illustrating a light-emitting apparatus of one embodiment.

FIG. 15A and FIG. 15B are diagrams illustrating a light-emitting apparatus of one embodiment.

FIG. 16A and FIG. 16B are diagrams illustrating a light-emitting apparatus of one embodiment.

FIG. 17A to FIG. 17E are diagrams illustrating electronic devices of one embodiment.

FIG. 18A to FIG. 18E are diagrams illustrating electronic devices of one embodiment.

FIG. 19A and FIG. 19B are diagrams illustrating an electronic device of one embodiment.

FIG. 20A and FIG. 20B are diagrams illustrating an electronic device of one embodiment.

FIG. 21 is a diagram illustrating electronic devices of one embodiment.

FIG. 22 is a ¹H-NMR chart of an organic compound represented by Structural Formula (100).

FIG. 23 shows an ultraviolet-visible absorption spectrum and an emission spectrum of the organic compound represented by Structural Formula (100) in a toluene solution

FIG. 24 shows an ultraviolet-visible absorption spectrum and an emission spectrum of the organic compound represented by Structural Formula (100) in a solid thin film state.

FIG. 25 shows a ¹H-NMR chart of an organic compound represented by Structural Formula (101).

FIG. 26 shows an ultraviolet-visible absorption spectrum and an emission spectrum of the organic compound represented by Structural Formula (101) in a toluene solution.

FIG. 27 shows an ultraviolet-visible absorption spectrum and an emission spectrum of the organic compound represented by Structural Formula (101) in a solid thin film state.

FIG. 28 is a ¹H-NMR chart of an organic compound represented by Structural Formula (102).

FIG. 29 shows an ultraviolet-visible absorption spectrum and an emission spectrum of the organic compound represented by Structural Formula (102) in a toluene solution.

FIG. 30 shows an ultraviolet-visible absorption spectrum and an emission spectrum of the organic compound represented by Structural Formula (102) in a solid thin film state.

FIG. 31 is a diagram illustrating a structure of a light-emitting device.

FIG. 32 shows an electroluminescence spectrum of a light-emitting device 1.

FIG. 33 shows an electroluminescence spectrum of a light-emitting device 2.

FIG. 34 shows an electroluminescence spectrum of a light-emitting device 3.

### MODE FOR CARRYING OUT THE INVENTION

Embodiments of the present invention will be described in detail below with reference to the drawings. Note that the present invention is not limited to the following description, and the modes and details of the present invention can be modified in various ways without departing from the spirit and scope of the present invention. Therefore, the present invention should not be construed as being limited to the description in the following embodiments.

Note that the position, size, range, or the like of each component shown in drawings and the like is not accurately represented in some cases for easy understanding. Therefore, the disclosed invention is not necessarily limited to the position, size, range, and the like disclosed in the drawings and the like.

Furthermore, when describing the structures of the invention with reference to the drawings in this specification and the like, the reference numerals denoting the same components are commonly used in different drawings.

In this specification and the like, a structure in which light-emitting layers in light-emitting devices of different colors (here, blue (B), green (G), and red (R)) are separately formed or separately patterned may be referred to as an SBS (Side By Side) structure. In this specification and the like, a light-emitting device capable of emitting white light is sometimes referred to as a white-light-emitting device. Note that a combination of white-light-emitting devices with coloring layers (e.g., color filters) enables a full-color display apparatus.

Structures of light-emitting devices can be classified roughly into a single structure and a tandem structure. A device with a single structure includes one light-emitting unit between a pair of electrodes, and the light-emitting unit preferably includes one or more light-emitting layers. In the case of obtaining white light emission with use of two light-emitting layers, the two light-emitting layers may be selected such that their emission colors are complementary colors. For example, when emission colors of a first light-emitting layer and a second light-emitting layer are complementary colors, the light-emitting device can be configured to emit white light as a whole. In the case of obtaining white light emission with use of three or more light-emitting layers, a light-emitting device may be configured to emit white color as a whole by combining colors emitted from the three or more light-emitting layers.

A device having a tandem structure includes two or more light-emitting units between a pair of electrodes, and each light-emitting unit preferably includes one or more light-emitting layers. To obtain white light emission, the structure is employed in which light from light-emitting layers of a plurality of light-emitting units is combined to enable white light emission. Note that a structure for obtaining white light emission is similar to that in the case of a single structure. In the device with a tandem structure, it is preferable that an intermediate layer such as a charge-generation layer be provided between the plurality of light-emitting units.

When the above white-light-emitting device (having a single structure or a tandem structure) and a light-emitting device having an SBS structure are compared to each other, the light-emitting device having an SBS structure can have lower power consumption than the white light-emitting device. To reduce power consumption, a light-emitting device having an SBS structure is suitably used. Meanwhile, the white-light-emitting device is suitable in terms of lower manufacturing cost or higher manufacturing yield because the manufacturing process of the white-light-emitting device is simpler than that of the light-emitting device having an SBS structure.

### Embodiment 1

In this embodiment, an organic compound of one embodiment of the present invention will be described.

The organic compound described in this embodiment has a structure represented by General Formula (G1) shown below.

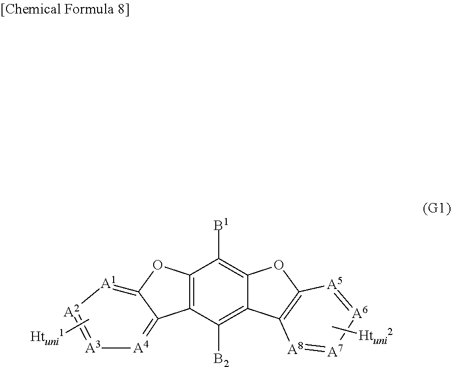

In General Formula (G1) shown above, at least one or two of A¹ to A⁴ represent nitrogen, and the others represent carbon. In addition, at least one or two of A⁵ to A⁸ represent nitrogen, and the others represent carbon. Furthermore, B¹ and B² each independently represent hydrogen, an alkyl group having 1 to 6 carbon atoms, or a cyano group. Furthermore, Htᵤₙᵢ¹ and Htᵤₙᵢ² each independently represent a hole-transport skeleton. Note that carbon in General Formula (G1) shown above may be bonded to hydrogen or a substituent.

In the above structure, Htᵤₙᵢ¹ and Htᵤₙᵢ² each independently have a carbazolyl group or an amino group.

In the above structure, it is preferable that Htᵤₙᵢ¹ and the Htᵤₙᵢ² be each independently a substituent represented by General Formula (Ht-1) or (Ht-2) below.

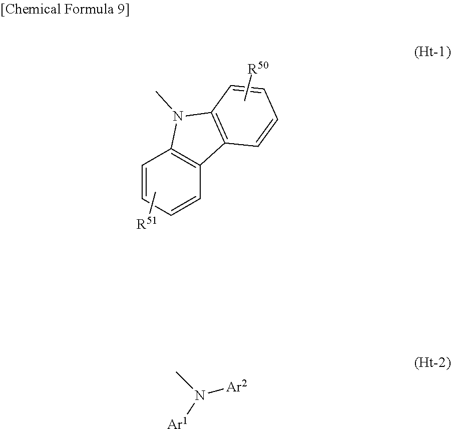

In General Formula (Ht-1) or (Ht-2) shown above, R⁵⁰ and R⁵¹ each represent 1 to 4 substituents and independently represent any one of hydrogen, an alkyl group having 1 to 6 carbon atoms, and a substituted or unsubstituted phenyl group. In addition, Ar¹ and Ar² represent any one of a substituted or unsubstituted phenyl group, a substituted or unsubstituted naphthyl group, a substituted or unsubstituted carbazolyl group, a substituted or unsubstituted fluorenyl group, a substituted or unsubstituted dibenzofuranyl group, and a substituted or unsubstituted dibenzothiophenyl group.

Another organic compound shown in this embodiment is represented by General Formula (G2) below.

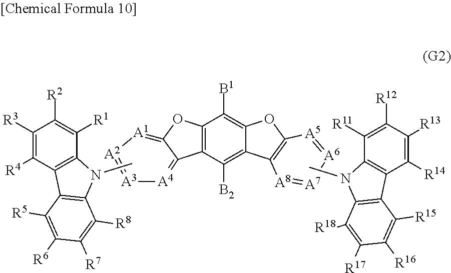

In General Formula (G2) shown above, at least one or two of A¹ to A⁴ represent nitrogen, and the others represent carbon. At least one or two of A⁵ to A⁸ represent nitrogen, and the others represent carbon. In addition, B¹ and B² each independently represent hydrogen, an alkyl group having 1 to 6 carbon atoms, or a cyano group. Furthermore, R¹ to R⁸ and R¹¹ to R¹⁸ each independently represent hydrogen, an alkyl group having 1 to 6 carbon atoms, a substituted or unsubstituted monocyclic saturated hydrocarbon group having 5 to 7 carbon atoms, a substituted or unsubstituted polycyclic saturated hydrocarbon group having 7 to 10 carbon atoms, a substituted or unsubstituted aryl group having 6 to 13 carbon atoms, or a substituted or unsubstituted heteroaryl group having 3 to 12 carbon atoms.

Another organic compound shown in this embodiment is represented by General Formula (G3) below.

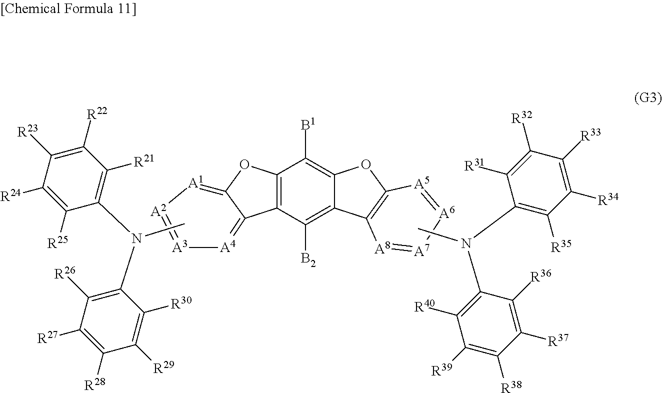

In General Formula (G3) shown above, at least one or two of A¹ to A⁴ represent nitrogen, and the others represent carbon. In addition, at least one or two of A⁵ to A⁸ represent nitrogen, and the others represent carbon. In addition, B¹ and B² each independently represent hydrogen, an alkyl group having 1 to 6 carbon atoms, or a cyano group. Furthermore, R²¹ to R³⁰ and R³¹ to R⁴⁰ each independently represent hydrogen, an alkyl group having 1 to 6 carbon atoms, a substituted or unsubstituted monocyclic saturated hydrocarbon group having 5 to 7 carbon atoms, a substituted or unsubstituted polycyclic saturated hydrocarbon group having 7 to 10 carbon atoms, a substituted or unsubstituted aryl group having 6 to 13 carbon atoms, or a substituted or unsubstituted heteroaryl group having 3 to 12 carbon atoms.

Another organic compound shown in this embodiment is represented by General Formula (G4) below.

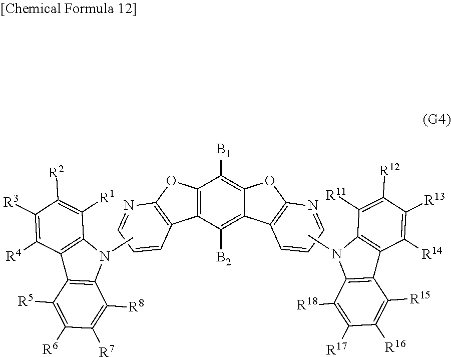

In General Formula (G4) shown above, B¹ and B² each independently represent hydrogen, an alkyl group having 1 to 6 carbon atoms, or a cyano group. Furthermore, R¹ to R⁸ and R¹¹ to R¹⁸ each independently represent hydrogen, an alkyl group having 1 to 6 carbon atoms, a substituted or unsubstituted monocyclic saturated hydrocarbon group having 5 to 7 carbon atoms, a substituted or unsubstituted polycyclic saturated hydrocarbon group having 7 to 10 carbon atoms, a substituted or unsubstituted aryl group having 6 to 13 carbon atoms, or a substituted or unsubstituted heteroaryl group having 3 to 12 carbon atoms.

In each of the above structures, R⁶ is preferably a monocyclic saturated hydrocarbon group having 3 to 20 carbon atoms. The monocyclic saturated hydrocarbon group having 3 to 20 carbon atoms is further preferably a cyclohexyl group.

Another organic compound shown in this embodiment is represented by General Formula (G5) below.

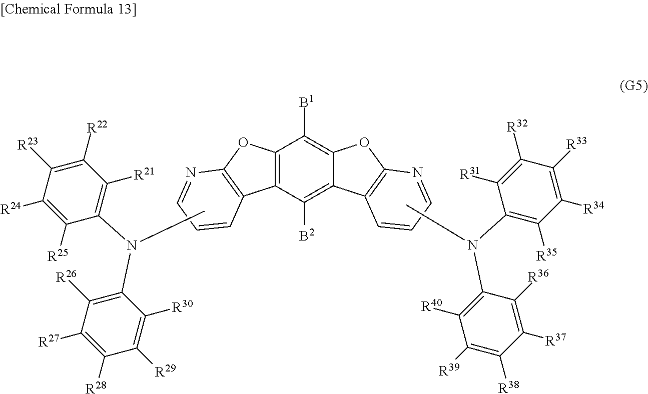

In General Formula (G5) shown above, B¹ and B² each independently represent hydrogen, an alkyl group having 1 to 6 carbon atoms, or a cyano group. Furthermore, R²¹ to R³⁰ and R³¹ to R⁴⁰ each independently represent hydrogen, an alkyl group having 1 to 6 carbon atoms, a substituted or unsubstituted monocyclic saturated hydrocarbon group having 5 to 7 carbon atoms, a substituted or unsubstituted polycyclic saturated hydrocarbon group having 7 to 10 carbon atoms, a substituted or unsubstituted aryl group having 6 to 13 carbon atoms, or a substituted or unsubstituted heteroaryl group having 3 to 12 carbon atoms.

In the case where a substituent is included in any of the substituted or unsubstituted aryl group having 6 to 13 carbon atoms, the substituted or unsubstituted monocyclic saturated hydrocarbon group having 5 to 7 carbon atoms, the substituted or unsubstituted polycyclic saturated hydrocarbon group having 7 to 10 carbon atoms, or a substituted or unsubstituted heteroaryl group having 3 to 12 carbon atoms in General Formulae (G2) to (G5) shown above or the substituted or unsubstituted phenyl group, the substituted or unsubstituted naphthyl group, the substituted or unsubstituted carbazolyl group, the substituted or unsubstituted fluorenyl group, the substituted or unsubstituted dibenzofuranyl group, or the substituted or unsubstituted dibenzothiophenyl group in General Formulae (Ht-1) and (Ht-2) shown above, examples of the substituent include an alkyl group having 1 to 7 carbon atoms such as a methyl group, an ethyl group, a propyl group, an isopropyl group, a butyl group, an isobutyl group, a sec-butyl group, a tert-butyl group, a pentyl group, a hexyl group, or a heptyl group, a cycloalkyl group having 5 to 7 carbon atoms such as a cyclopentyl group, a cyclohexyl group, a cycloheptyl group, or a 8,9,10-trinorbornanyl group, and an aryl group having 6 to 12 carbon atoms such as a phenyl group, a naphthyl group, or a biphenyl group.

Specific examples of the monocyclic saturated hydrocarbon group having 5 to 7 carbon atoms in General Formulae (G2) to (G5) shown above include a cyclopentyl group, a cyclohexyl group, a cycloheptyl group, and a 2-methylcyclohexyl group.

Specific examples of the polycyclic saturated hydrocarbon group having 7 to 10 carbon atoms in General Formulae (G2) to (G5) shown above include a 8,9,10-trinorbornanyl group, a decahydronaphthyl group, and an adamantyl group.

Specific examples of the aryl group having 6 to 13 carbon atoms in General Formulae (G2) to (G5) shown above are a phenyl group, an o-tolyl group, an m-tolyl group, a p-tolyl group, a mesityl group, an o-biphenyl group, an m-biphenyl group, a p-biphenyl group, a 1-naphthyl group, a 2-naphthyl group, a fluorenyl group, a 9,9-dimethylfluorenyl group, and the like.

Specific examples of the alkyl group having 1 to 6 carbon atoms in General Formulae (G1) to (G6) shown above are a methyl group, an ethyl group, a propyl group, an isopropyl group, a butyl group, a sec-butyl group, an isobutyl group, a tert-butyl group, a pentyl group, an isopentyl group, a sec-pentyl group, a tert-pentyl group, a neopentyl group, a hexyl group, an isohexyl group, a 3-methylpentyl group, a 2-methylpentyl group, a 2-ethylbutyl group, a 1,2-dimethylbutyl group, a 2,3-dimethylbutyl group, and the like.

Next, specific structural formulae of the above organic compound of one embodiment of the present invention are shown below. Note that the present invention is not limited to these formulae.

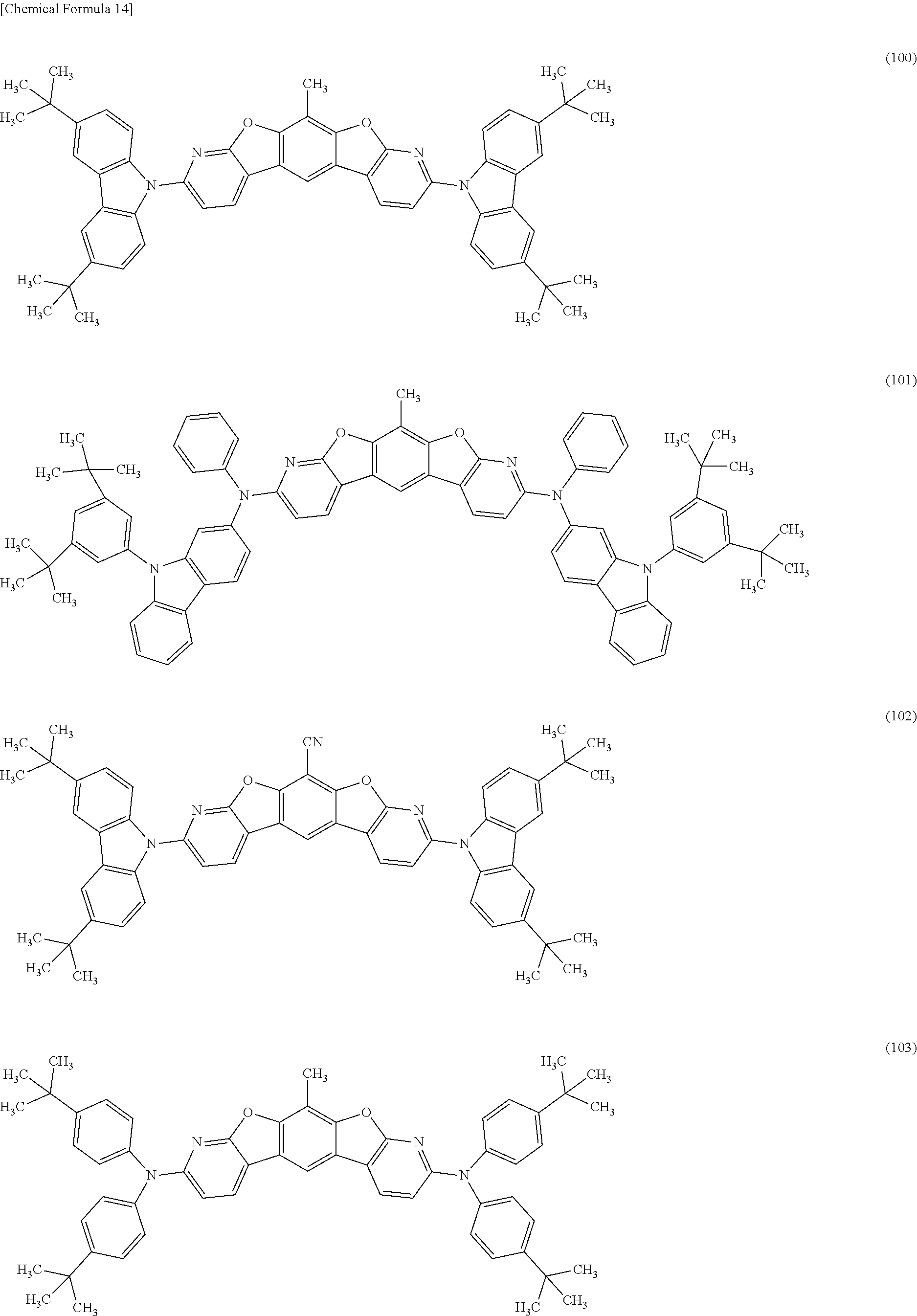

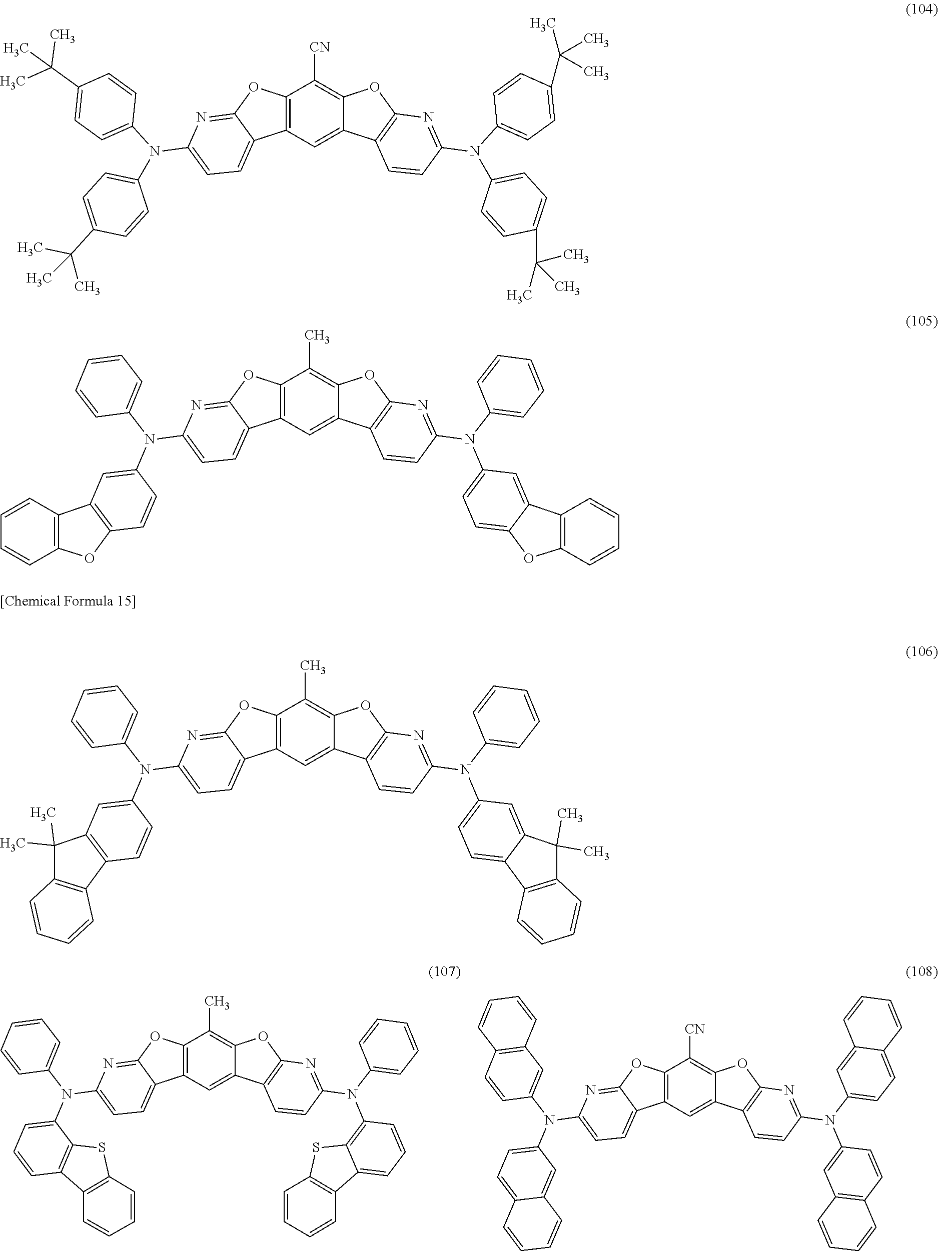

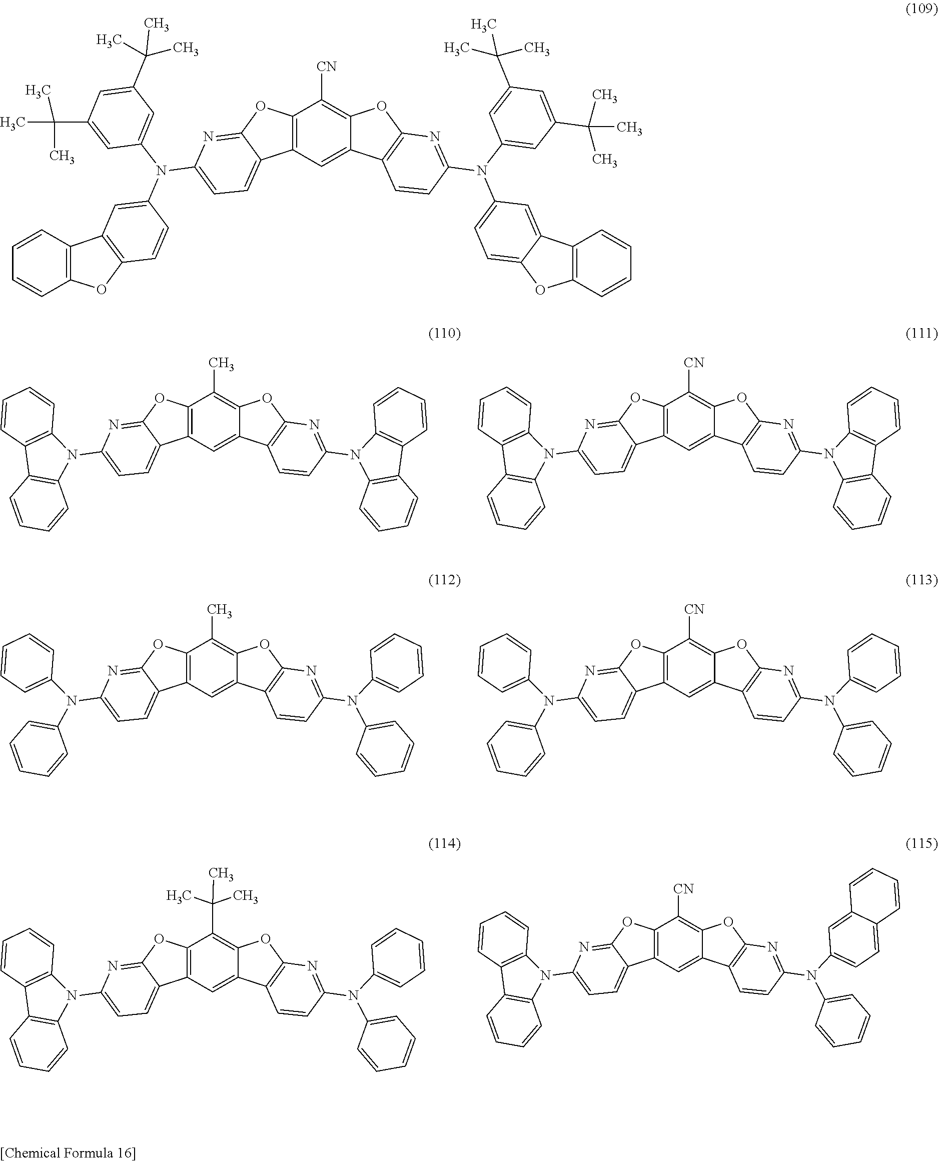

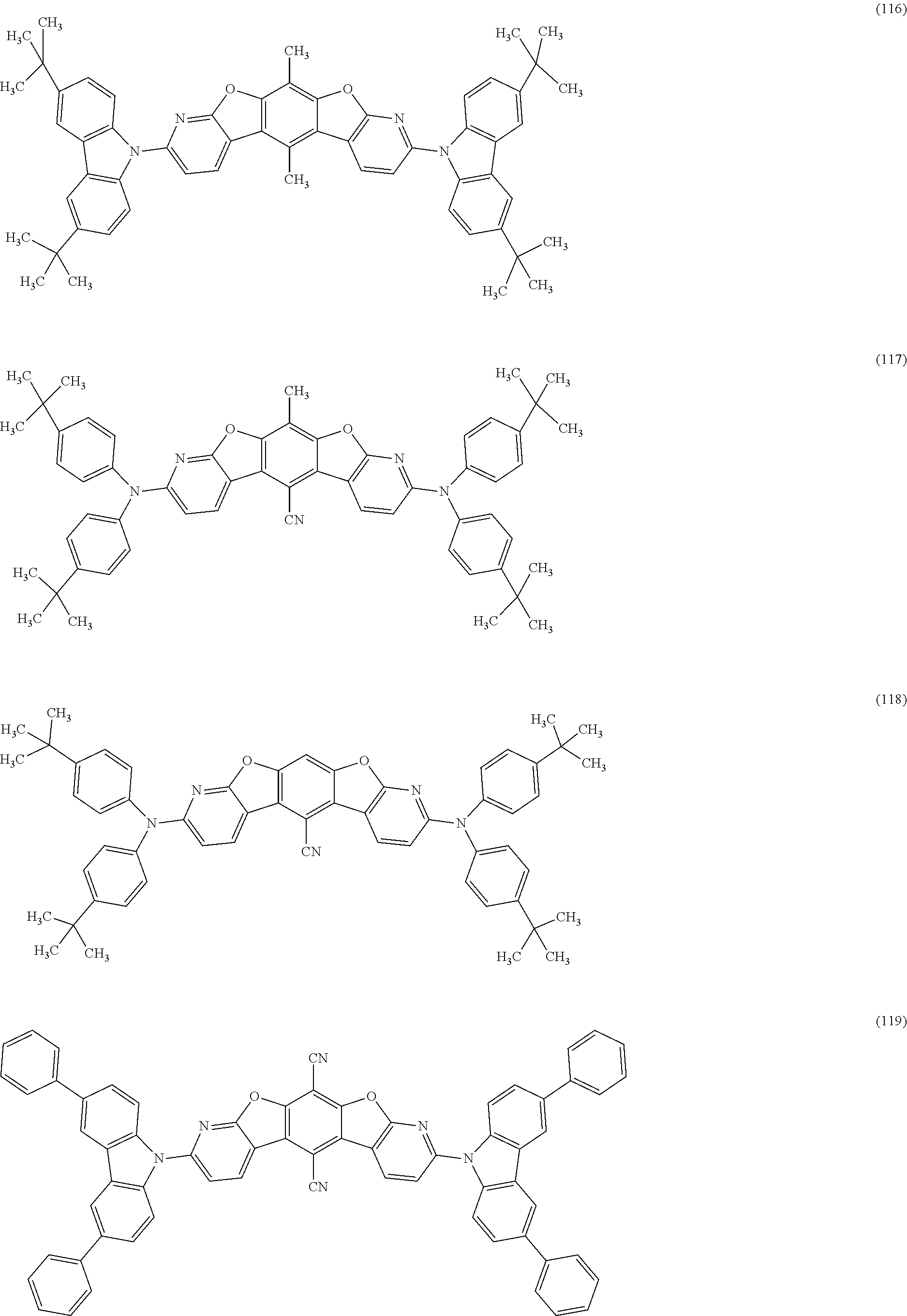

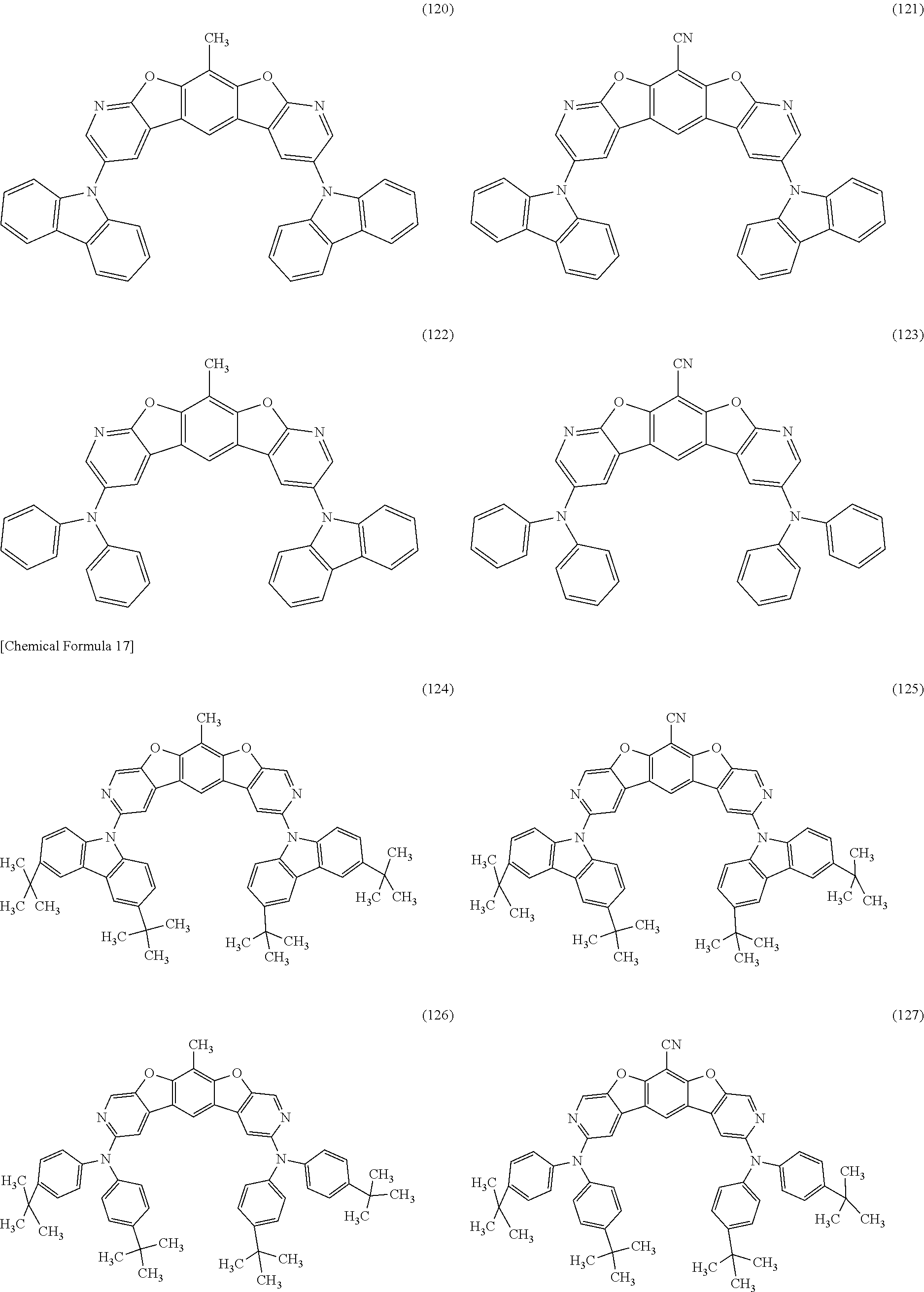

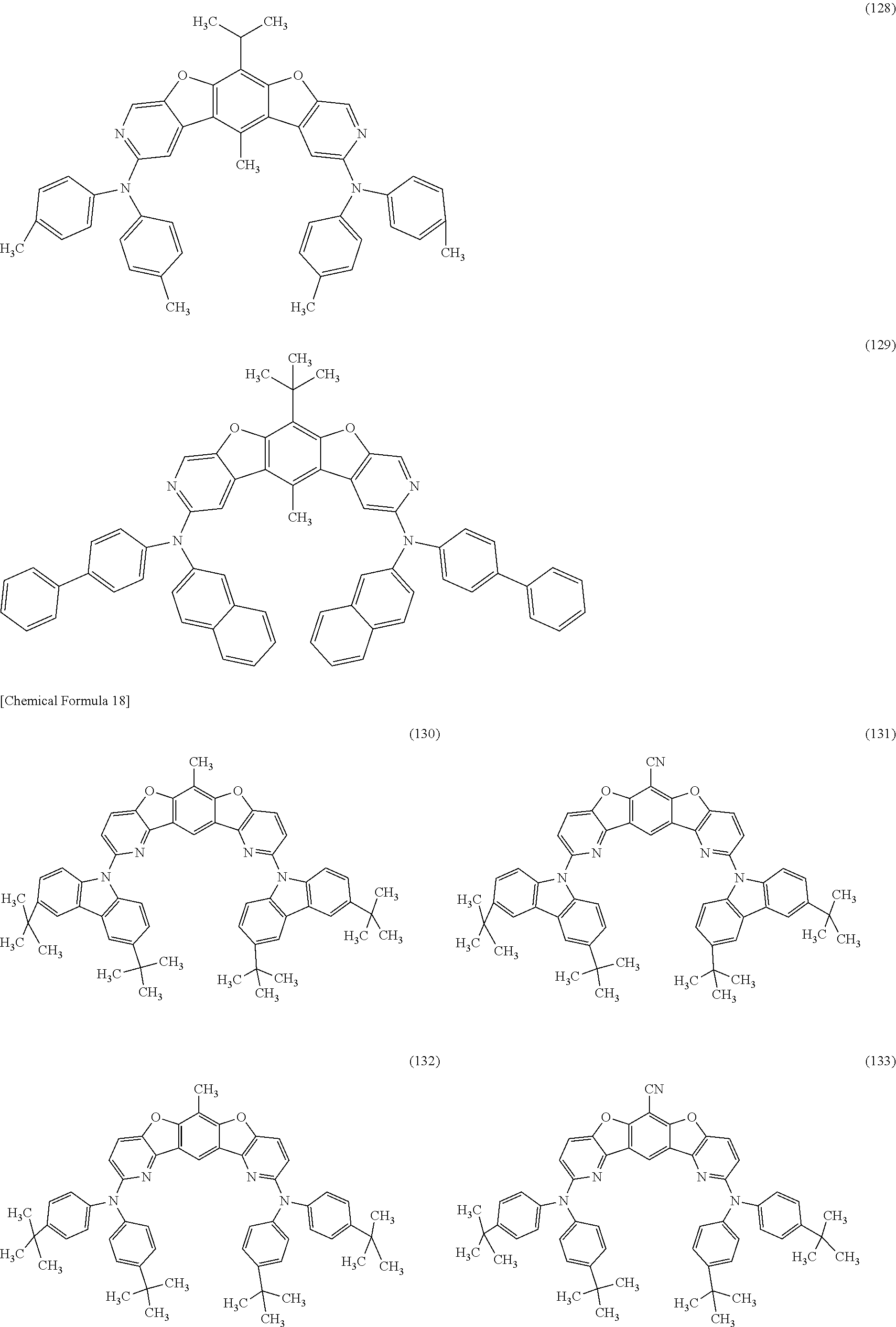

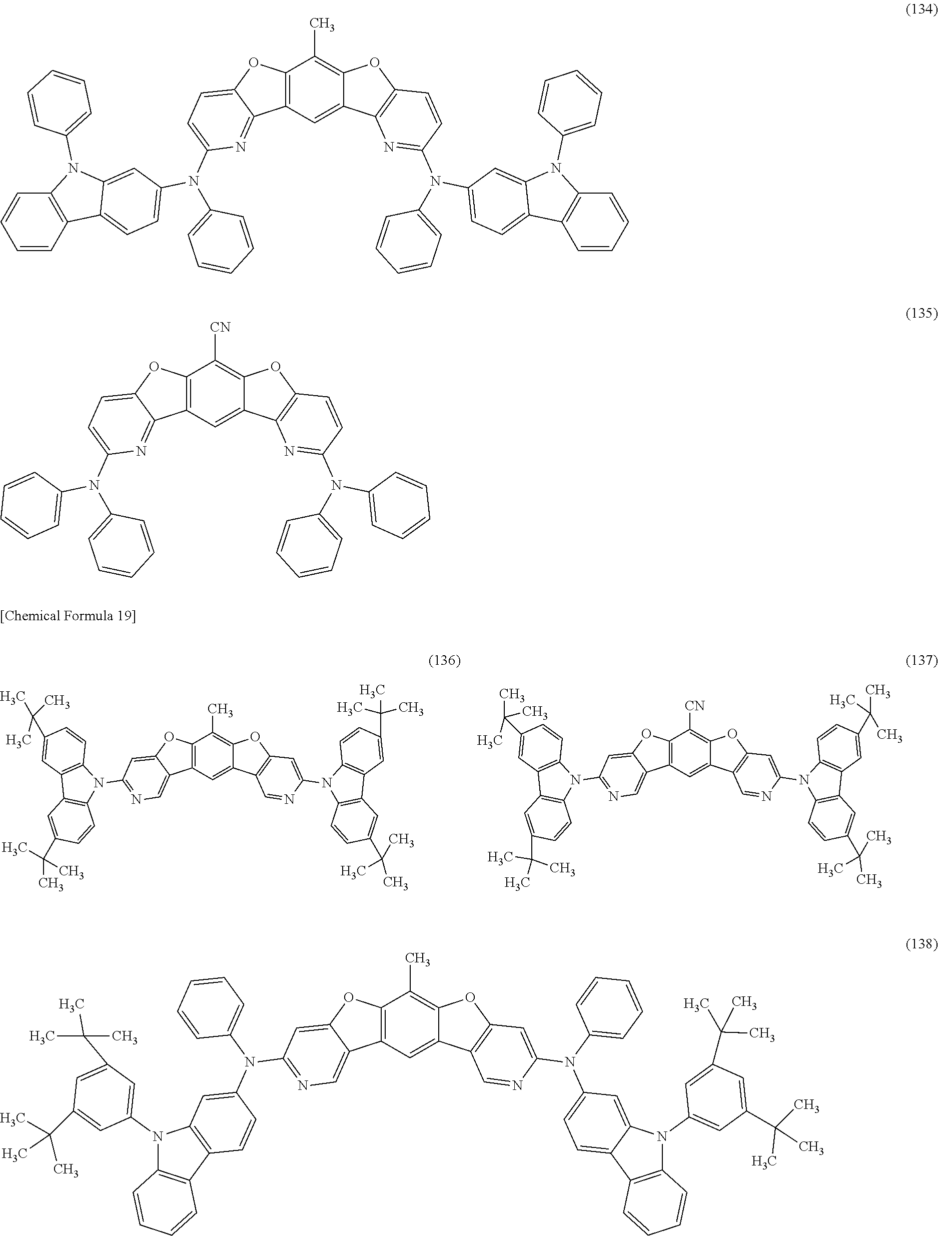

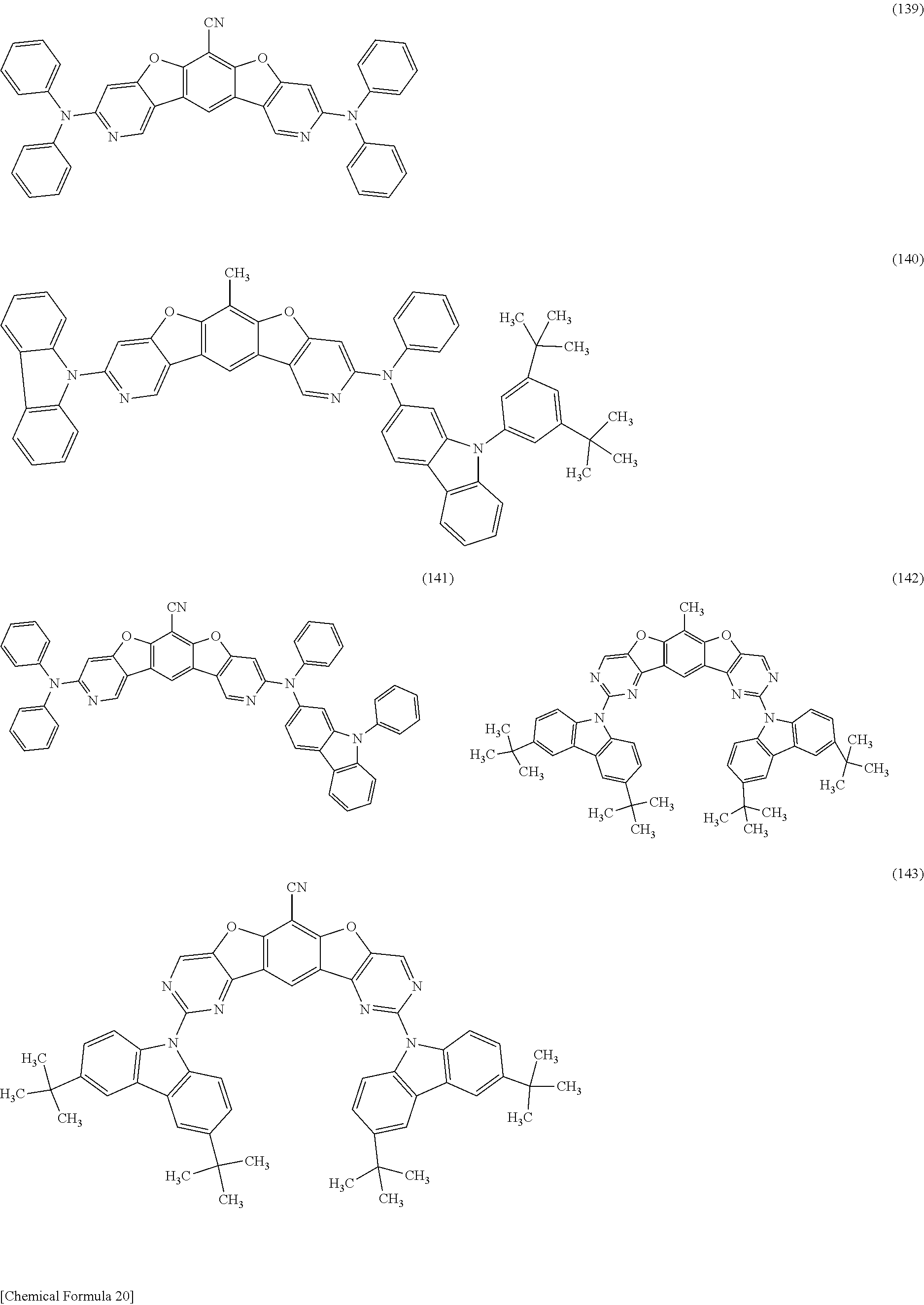

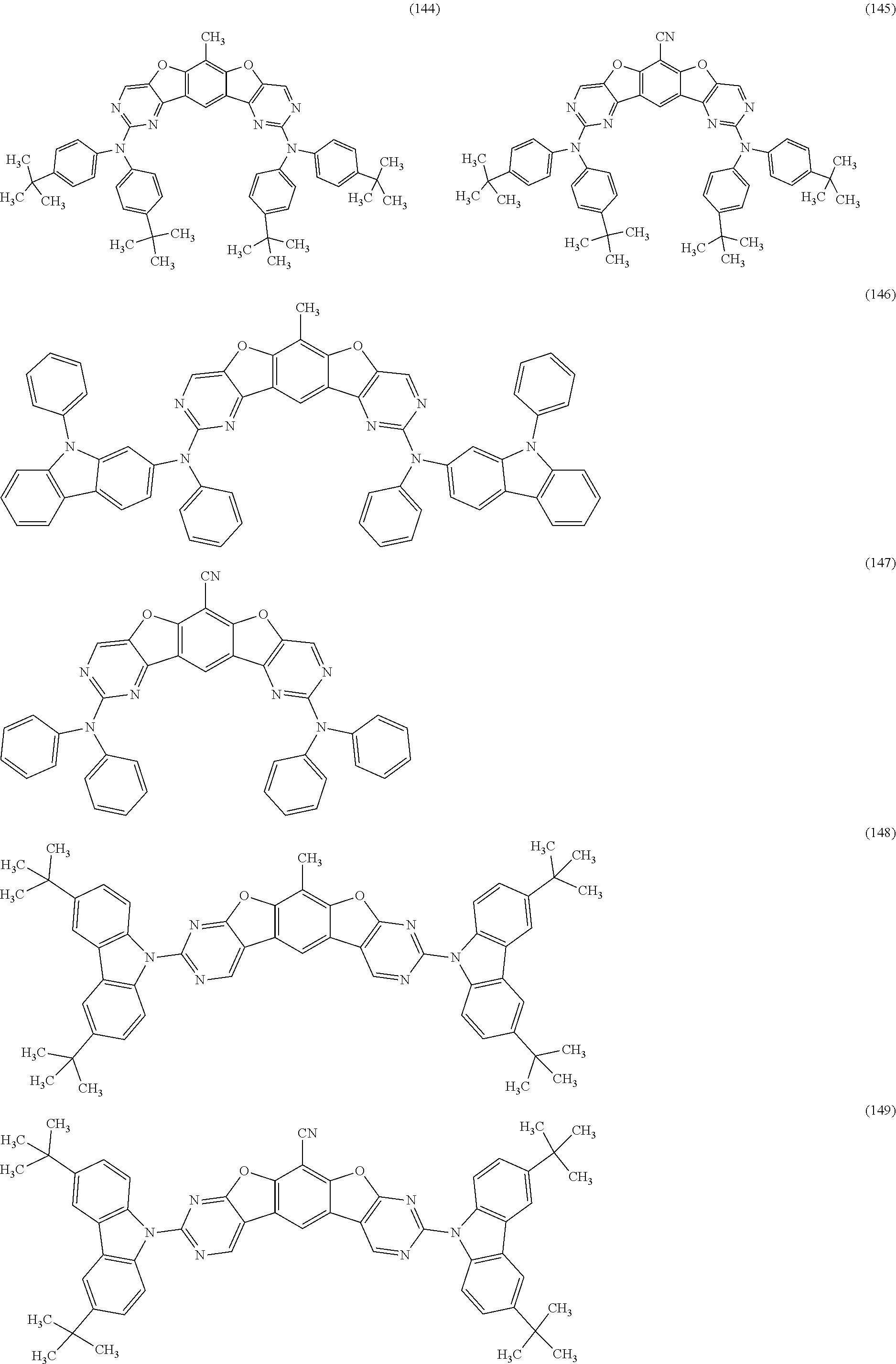

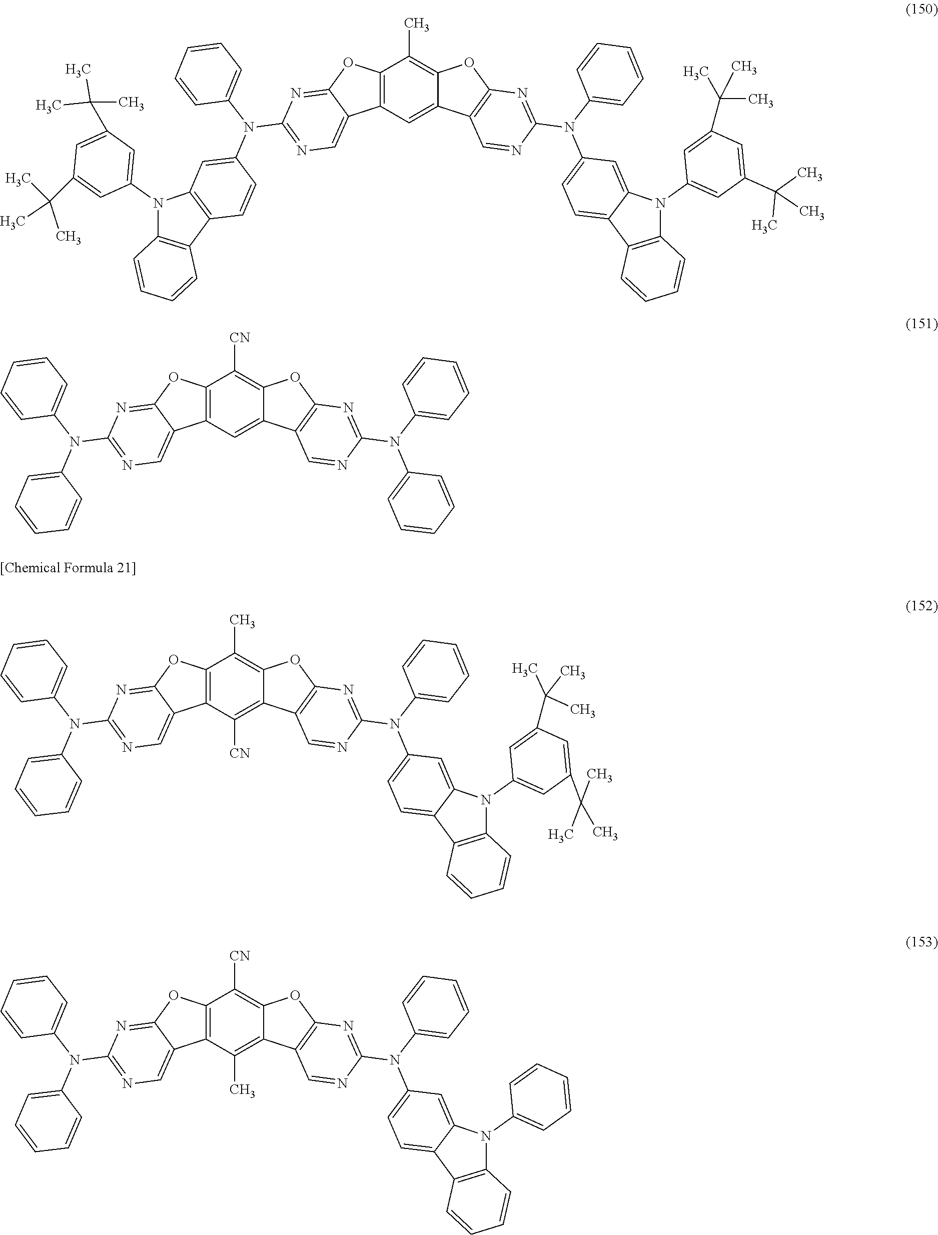 [SMILES: Cc1c2oc3nc(N(c4ccccc4)c4ccc5c6ccccc6n(-c6cc(C(C)(C)C)cc(C(C)(C)C)c6)c5c4)ncc3c2cc2c1oc1nc(N(c3ccccc3)c3ccc4c5ccccc5n(-c5cc(C(C)(C)C)cc(C(C)(C)C)c5)c4c3)ncc12 ; N#Cc1c2oc3nc(N(c4ccccc4)c4ccccc4)ncc3c2cc2c1oc1nc(N(c3ccccc3)c3ccccc3)ncc12 ; Cc1c2oc3nc(N(c4ccccc4)c4ccccc4)ncc3c2c(C#N)c2c1oc1nc(N(c3ccccc3)c3ccc4c5ccccc5n(-c5cc(C(C)(C)C)cc(C(C)(C)C)c5)c4c3)ncc12 ; Cc1c2c(oc3nc(N(c4ccccc4)c4ccccc4)ncc32)c(C#N)c2oc3nc(N(c4ccccc4)c4ccc5c6ccccc6n(-c6ccccc6)c5c4)ncc3c12]

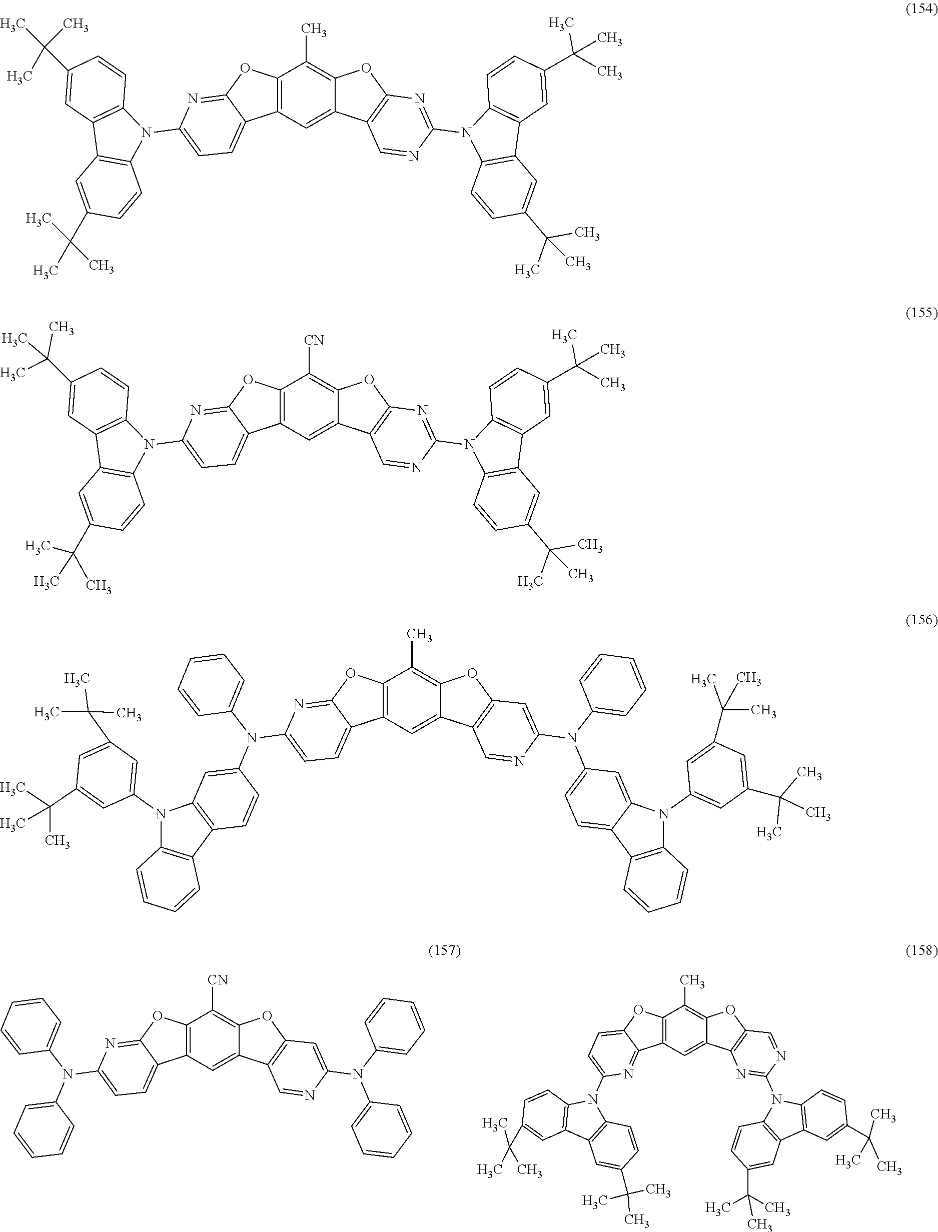

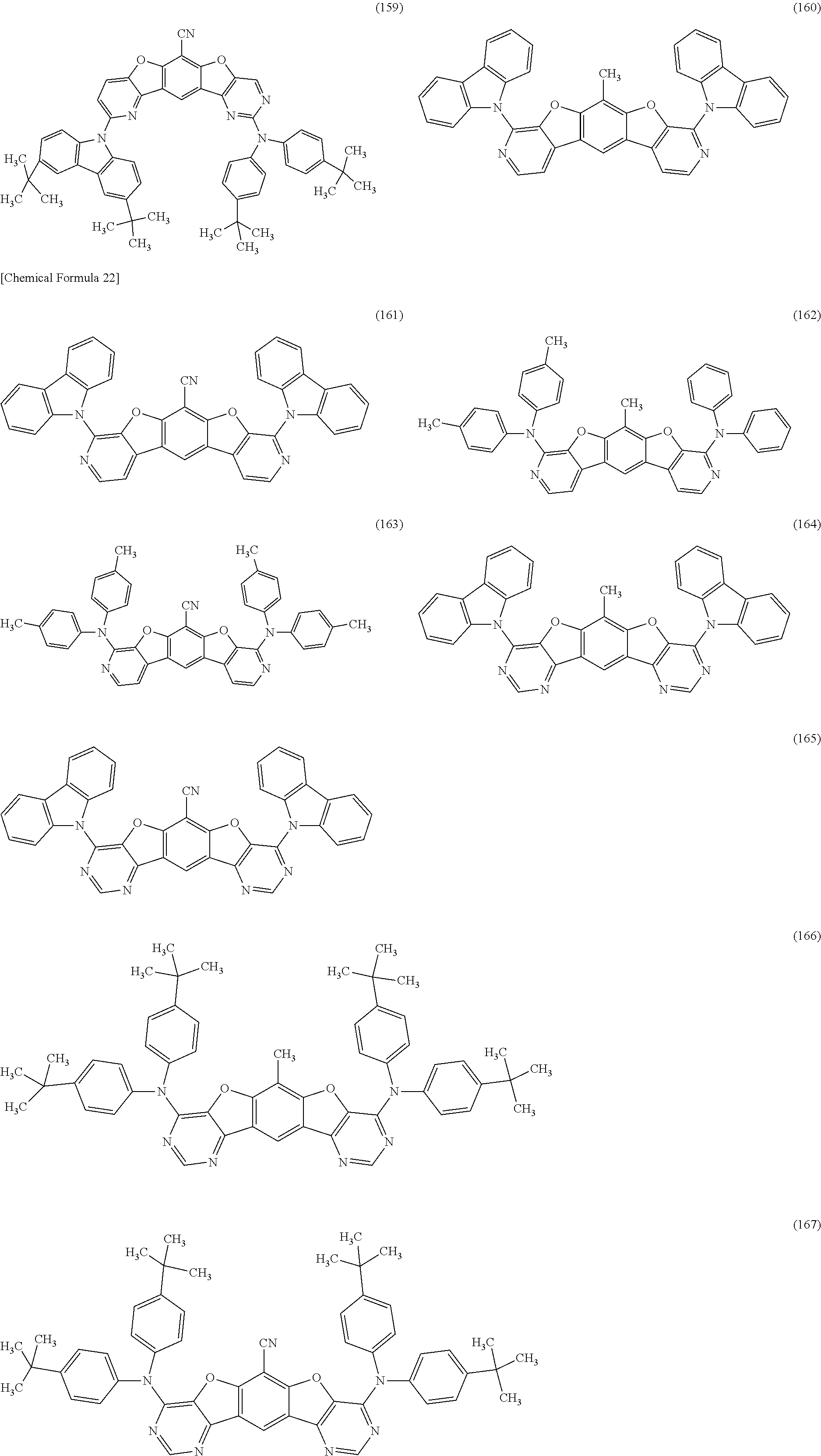

Note that the organic compounds represented by Structural Formulae (100) to (167) shown above are examples of the organic compounds represented by General Formulae (G1) to (G5) shown above. The organic compound of one embodiment of the present invention is not limited thereto.

Next, an example of a method for synthesizing the organic compound of one embodiment of the present invention, which is represented by General Formula (G1), will be described.

### <<Method for Synthesizing Organic Compound Represented by General Formula (G1)>>

An example of a method for synthesizing the organic compound represented by General Formula (G1) below is described.

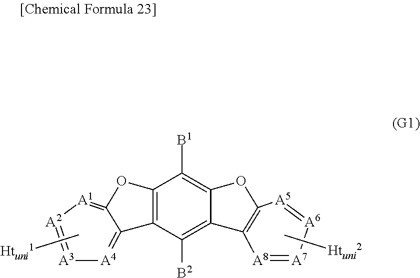

In General Formula (G1) shown above, at least one or two of A¹ to A⁴ represent nitrogen, and the others represent carbon. In addition, at least one or two of A⁵ to A⁸ represent nitrogen, and the others represent carbon. Furthermore, B¹ and B² each independently represent hydrogen, an alkyl group having 1 to 6 carbon atoms, or a cyano group. Furthermore, Htᵤₙᵢ¹ and Htᵤₙᵢ² each independently represent a hole-transport skeleton and has any of a carbazolyl group or an amino group.

First, as shown in Synthesis Scheme (A-1) below, a substituted benzene having B¹, B², Q¹, and Q² (a compound 1), a substituted hetero six-membered cyclic compound having Q³ and X¹ (a compound 2), and a substituted hetero six-membered cyclic compound having Q⁴ and X¹ (a compound 3) are reacted to obtain a compound 4.

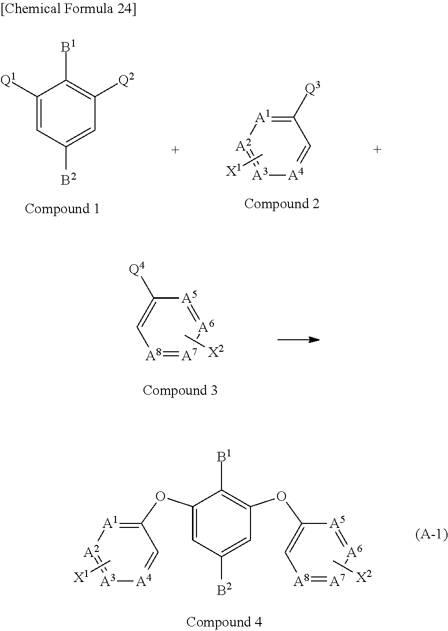

Note that in Synthesis Scheme (A-1) shown above, at least one or two of A¹ to A⁴ represent nitrogen, and the others represent carbon. In addition, at least one or two of A⁵ to A⁸ represent nitrogen, and the others represent carbon. Furthermore, B¹ and B² each independently represent hydrogen, an alkyl group having 1 to 6 carbon atoms, or a cyano group. Furthermore, one of Q¹ and Q³ represents a halogen, and the other represents a hydroxy group. Furthermore, one of Q² and Q⁴ represents a halogen, and the other represents a hydroxy group. Furthermore, X¹ and X² each represent a halogen.

The reaction shown in Synthesis Scheme (A-1) above may be performed under the presence of a base. Potassium carbonate, cesium carbonate, or the like can be used as the base. Furthermore, N,N-dimethylformamide (DMF), toluene, xylene, mesitylene, benzene, tetrahydrofuran, dioxane, or the like can be used as a solvent. Reagents that can be used in the reaction are not limited thereto.

In the case where the compound 2 and the compound 3 have different structures in Synthesis Scheme (A-1) above, it is preferable that the compound 1 and the compound 2 be reacted first to form a product and then the product obtained through the reaction and the compound 3 be reacted. In the case where the compound 1 is reacted with the compound 2 and the compound 3 in different stages, it is preferable that Q¹ and Q² be different halogens or hydroxy groups and selectively subjected to reactions.

In Synthesis Scheme (A-1) shown above, it is preferable that one of Q¹ and Q³ and one of Q² and Q⁴ be halogens having higher reactivity than X¹ and X² and selectively subjected to reactions. For example, in the case where X¹ and X² are chlorine, bromine, or iodine, fluorine is used for one of Q¹ and Q³ and one of Q² and Q⁴, whereby selective reaction can be made. The compound 4 is synthesized in Synthesis Scheme (A-1) shown above, whereby a compound 5 can be easily obtained through intermolecular carbon-hydrogen (C—H) binding activity reaction in subsequent Synthesis Scheme (A-2). Note that in Synthesis Scheme (A-2), it is preferable that X¹ and X² in the compound 4 be chlorine, in which case the compound 5 can be selectively synthesized.

Specific examples of the compound 4 shown in Synthesis Scheme (A-1) include those which are represented by Structural Formulae (200) to (229) below.

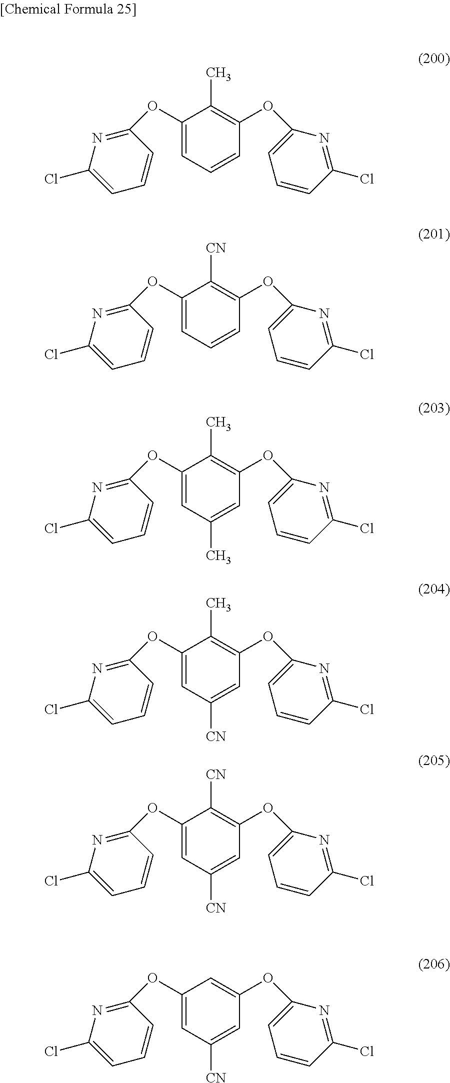

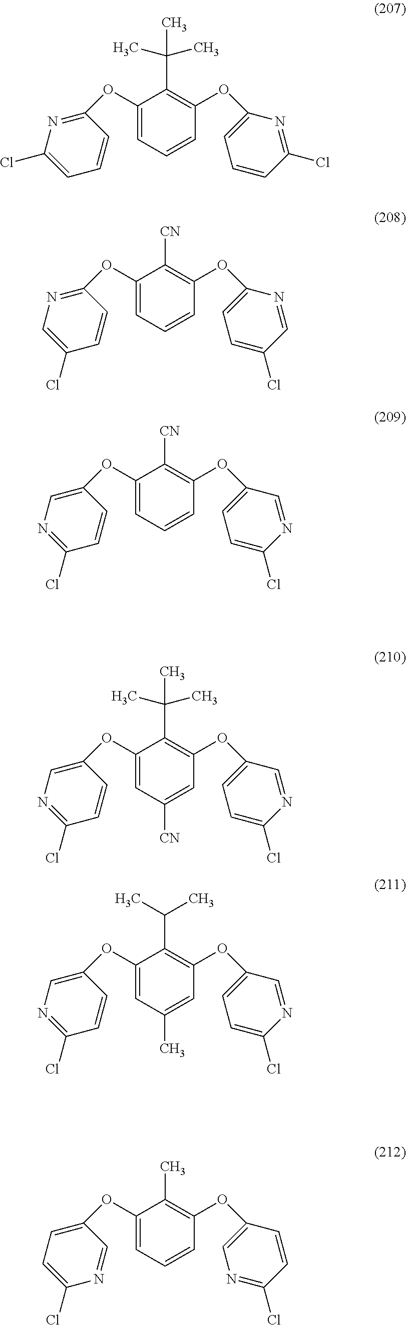 [SMILES: CC(C)(C)c1c(Oc2cccc(Cl)n2)cccc1Oc1cccc(Cl)n1 ; N#Cc1c(Oc2ccc(Cl)cn2)cccc1Oc1ccc(Cl)cn1 ; N#Cc1c(Oc2ccc(Cl)nc2)cccc1Oc1ccc(Cl)nc1 ; CC(C)(C)c1c(Oc2ccc(Cl)nc2)cc(C#N)cc1Oc1ccc(Cl)nc1 ; Cc1cc(Oc2ccc(Cl)nc2)c(C(C)C)c(Oc2ccc(Cl)nc2)c1 ; Cc1c(Oc2ccc(Cl)nc2)cccc1Oc1ccc(Cl)nc1]

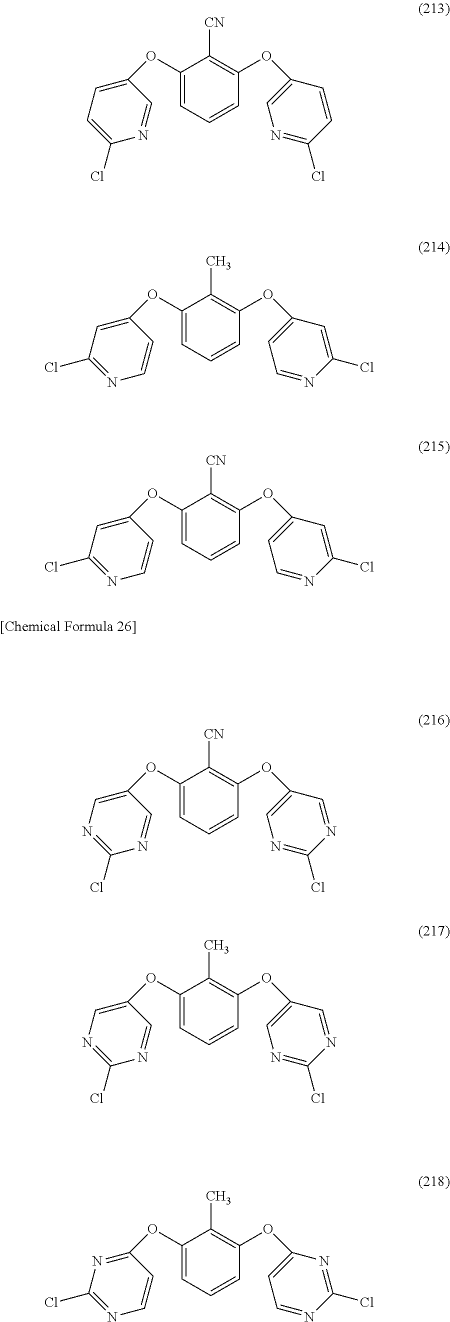 [SMILES: N#Cc1c(Oc2ccc(Cl)nc2)cccc1Oc1ccc(Cl)nc1 ; Cc1c(Oc2ccnc(Cl)c2)cccc1Oc1ccnc(Cl)c1 ; N#Cc1c(Oc2ccnc(Cl)c2)cccc1Oc1ccnc(Cl)c1 ; N#Cc1c(Oc2cnc(Cl)nc2)cccc1Oc1cnc(Cl)nc1 ; Cc1c(Oc2cnc(Cl)nc2)cccc1Oc1cnc(Cl)nc1 ; Cc1c(Oc2ccnc(Cl)n2)cccc1Oc1ccnc(Cl)n1]

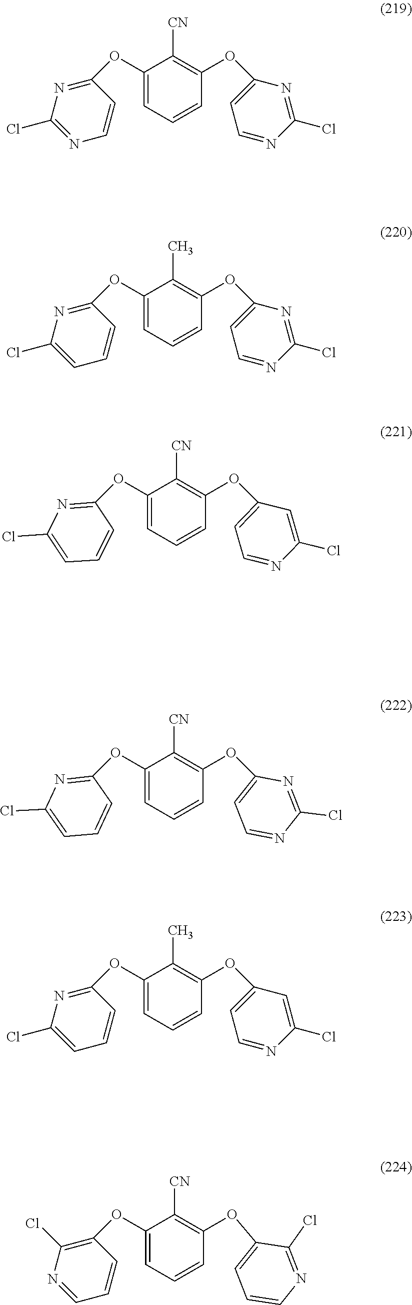 [SMILES: N#Cc1c(Oc2ccnc(Cl)n2)cccc1Oc1ccnc(Cl)n1 ; Cc1c(Oc2cccc(Cl)n2)cccc1Oc1ccnc(Cl)n1 ; N#Cc1c(Oc2ccnc(Cl)c2)cccc1Oc1cccc(Cl)n1 ; N#Cc1c(Oc2cccc(Cl)n2)cccc1Oc1ccnc(Cl)n1 ; Cc1c(Oc2ccnc(Cl)c2)cccc1Oc1cccc(Cl)n1 ; N#Cc1c(Oc2cccnc2Cl)cccc1Oc1cccnc1Cl]

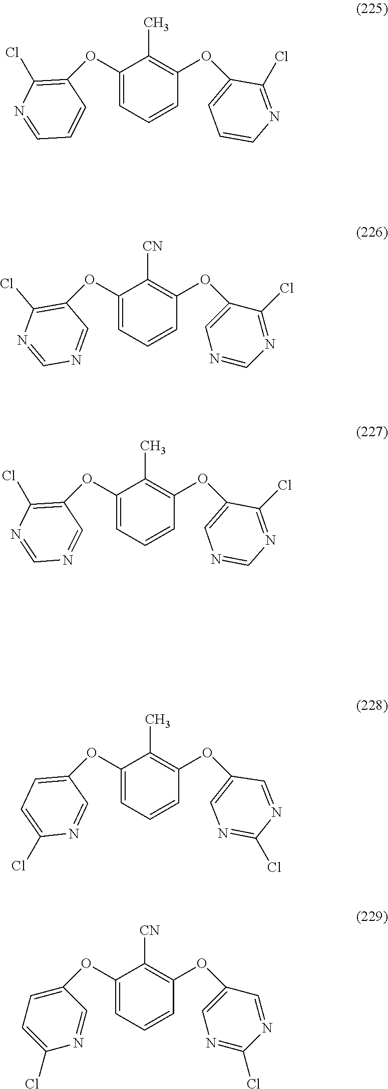 [SMILES: Cc1c(Oc2cccnc2Cl)cccc1Oc1cccnc1Cl ; N#Cc1c(Oc2cncnc2Cl)cccc1Oc1cncnc1Cl ; Cc1c(Oc2cncnc2Cl)cccc1Oc1cncnc1Cl ; Cc1c(Oc2ccc(Cl)nc2)cccc1Oc1cnc(Cl)nc1 ; N#Cc1c(Oc2ccc(Cl)nc2)cccc1Oc1cnc(Cl)nc1]

Next, as shown in Synthesis Scheme (A-2) below, the compound 5 is obtained from the compound 4 through the intermolecular carbon-hydrogen (C—H) binding activity reaction using a transition metal catalyst.

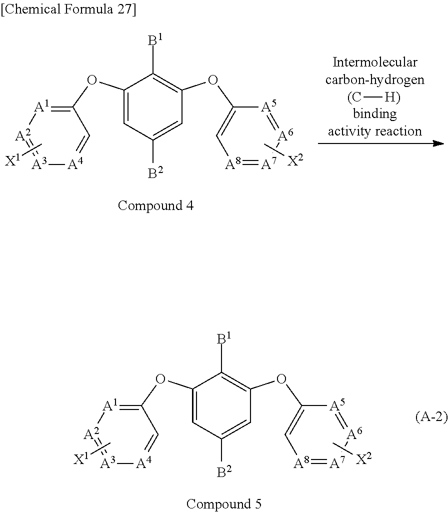

In the Synthesis Scheme (A-2) shown above, at least one or two of A¹ to A⁴ represent nitrogen, and the others represent carbon. In addition, at least one or two of A⁵ to A⁸ represent nitrogen, and the others represent carbon. Furthermore, B¹ and B² each independently represent hydrogen, an alkyl group having 1 to 6 carbon atoms, or a cyano group. Furthermore, X¹ and X² each represent a halogen.

In Synthesis Scheme (A-2) shown above, palladium acetate, trifluoroacetic palladium acetate, and the like can be used as the transition metal catalyst. Alternatively, as another transition metal catalyst, tetrakis(triphenylphosphine)palladium, dichlorobis(triphenylphosphine)palladium, or tris(dibenzylideneacetone)dipalladium may be used. The reaction shown in Synthesis Scheme (A-2) above may be performed under the presence of an oxidizer. As the oxidizer, silver acetate, silver trifluoroacetate, pivalic acid silver, or the like can be used. Furthermore, pivalic acid, N,N-dimethylformamide (DMF), toluene, xylene, mesitylene, benzene, tetrahydrofuran, dioxane, or the like can be used as a solvent. Reagents that can be used in the reaction are not limited thereto.

As described above, in Synthesis Scheme (A-2) shown above, it is further preferable that X¹ and X² be chlorine, in which case the compound 5 can be selectively synthesized.

Next, as shown in Scheme (A-3) below, the compound 5 is subjected to coupling reaction with a carbazole compound or an amine compound (Y¹-Htᵤₙᵢ¹ and Y²-Htᵤₙᵢ²), whereby an organic compound represented by General Formula (G1) shown above can be obtained.

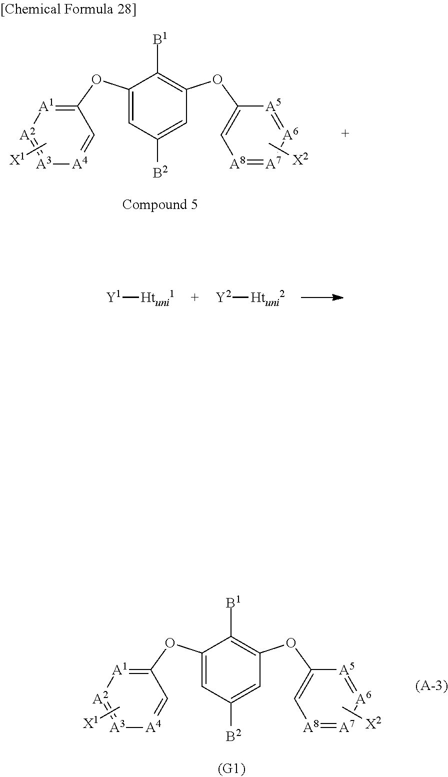

In Synthesis Scheme (A-3) shown above, at least one or two of A¹ to A⁴ represent nitrogen, and the others represent carbon. In addition, at least one or two of A⁵ to A⁸ represent nitrogen, and the others represent carbon. Furthermore, B¹ and B² each independently represent hydrogen, an alkyl group having 1 to 6 carbon atoms, or a cyano group. Furthermore, Htᵤₙᵢ¹ and Htᵤₙᵢ² each independently represent a hole-transport skeleton and has any of a carbazolyl group or an amino group. Furthermore, Y¹ and Y² each represent hydrogen, an organotin group, or the like.

The above reaction shown in Synthesis Scheme (A-3) can proceed under various conditions. For example, a synthesis method in which a metal catalyst is used under the presence of a base can be employed. For example, Ullmann coupling or the Buchwald-Hartwig reaction can be used. For example, in the case of using the Buchwald-Hartwig reaction, a palladium compound such as bis(dibenzylideneacetone)palladium(0) or palladium(II) acetate and a ligand such as tri(tert-butyl)phosphine, tri(n-hexyl)phosphine, tricyclohexylphosphine, di(1-adamantyl)-n-butylphosphine, or 2-dicyclohexylphosphino-2′,6′-dimethoxy-1,1′-biphenyl can be used as a metal catalyst. An organic base such as sodium tert-butoxide, an inorganic base such as potassium carbonate, cesium carbonate, or sodium carbonate, or the like can be used as the base. Furthermore, toluene, xylene, mesitylene, benzene, tetrahydrofuran, dioxane, or the like can be used as a solvent. Reagents that can be used in the reaction are not limited thereto.

When Y¹-Htᵤₙᵢ¹ and Y²-Htᵤₙᵢ² have different structure in Synthesis Scheme (A-3) shown above, it is preferable that the compound 5 and Y¹-Htᵤₙᵢ¹ be reacted first to form a product and then the product obtained through the reaction be reacted with Y²-Htᵤₙᵢ². In the case where the compound 5 is reacted with Y¹-Htᵤₙᵢ¹ and Y²-Htᵤₙᵢ² in different stages, it is preferable that X¹ and X² be different halogens and selectively subjected to reactions.

Since a wide variety kind of compound 1, the compound 2, the compound 3, and the compound 4 are commercially available or their synthesis is feasible, a great variety of the organic compounds represented by General Formula (G1) can be synthesized. Thus, the organic compound of one embodiment of the present invention is characterized by having numerous variations.

Described above are the methods for synthesizing the organic compound of one embodiment of the present invention and is represented by General Formula (G1); however, the present invention is not limited thereto and the organic compound may be synthesized by another synthesis method.

With use of the organic compound of one embodiment of the present invention, a light-emitting device, a light-emitting apparatus, an electronic device, or a lighting device with high emission efficiency can be obtained. In addition, a light-emitting device, a light-emitting apparatus, an electronic device, or a lighting device with low power consumption can be achieved.

Note that, in this embodiment, one embodiment of the present invention has been described. Other embodiments of the present invention are described in the other embodiments. However, one embodiment of the present invention is not limited thereto. In other words, since various embodiments of the invention are described in this embodiment and the other embodiments, embodiments of the present invention are not limited to particular embodiments.

The structures described in this embodiment can be used in an appropriate combination with the structures described in the other embodiments.

### Embodiment 2

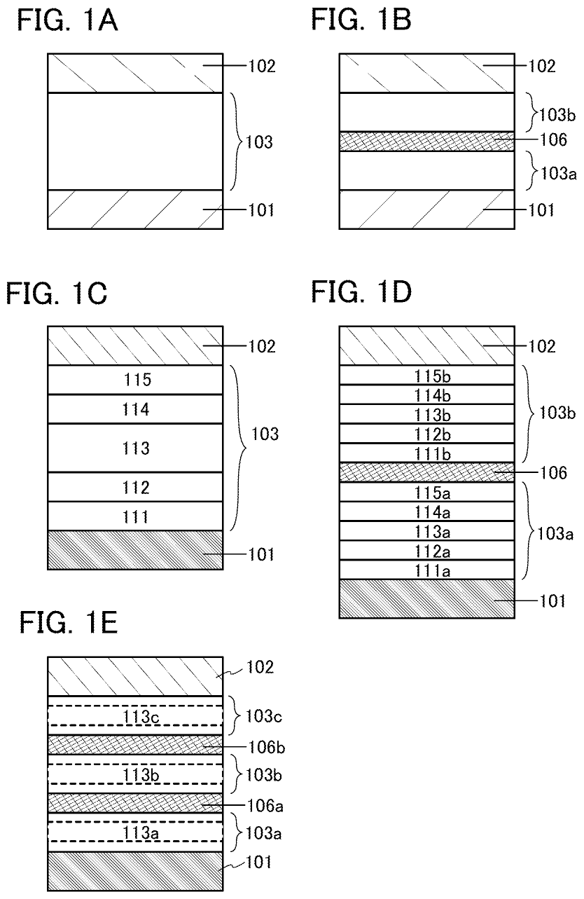

In this embodiment, a light-emitting device using the organic compound described in Embodiment 1 will be described with reference to FIG. 1A to FIG. 1E.

### <<Specific Structure of Light-Emitting Device>>

Among light-emitting devices shown in FIG. 1A to FIG. 1E, the light-emitting devices shown in FIG. 1A and FIG. 1C each have a structure in which an EL layer is sandwiched between a pair of electrodes, whereas the light-emitting devices shown in FIG. 1B, FIG. 1D, and FIG. 1E each have a structure in which, between a pair of electrodes, two or more EL layers are stacked with a charge-generation layer positioned therebetween (a tandem structure). Note that the structure of the EL layer is common between these structures. In the case where the light-emitting element in FIG. 1D has a microcavity structure, a first electrode 101 is formed as a reflective electrode and a second electrode 102 is formed as a transflective electrode. Thus, a single-layer structure or a stacked-layer structure can be formed using one or more kinds of desired electrode materials. Note that the second electrode 102 is formed after formation of an EL layer 103b, with the use of a material selected as described above.

### <First Electrode and Second Electrode>

As materials for forming the first electrode 101 and the second electrode 102, any of the following materials can be used in an appropriate combination as long as the functions of the electrodes described above can be fulfilled. For example, a metal, an alloy, an electrically conductive compound, and a mixture of these can be used as appropriate. Specifically, an In—Sn oxide (also referred to as ITO), an In—Si—Sn oxide (also referred to as ITSO), an In—Zn oxide, or an In—W—Zn oxide can be given. It is also possible to use a metal such as aluminum (Al), titanium (Ti), chromium (Cr), manganese (Mn), iron (Fe), cobalt (Co), nickel (Ni), copper (Cu), gallium (Ga), zinc (Zn), indium (In), tin (Sn), molybdenum (Mo), tantalum (Ta), tungsten (W), palladium (Pd), gold (Au), platinum (Pt), silver (Ag), yttrium (Y), or neodymium (Nd) or an alloy containing an appropriate combination of any of these metals. It is also possible to use an element belonging to Group 1 or Group 2 in the periodic table, which is not listed above as an example (e.g., lithium (Li), cesium (Cs), calcium (Ca), or strontium (Sr)), a rare earth metal such as europium (Eu) or ytterbium (Yb), an alloy containing an appropriate combination of any of these elements, graphene, or the like.

When the first electrode 101 is an anode in the light-emitting device illustrated in FIG. 1A and FIG. 1C, an EL layer 103 is formed over the first electrode 101 by a vacuum evaporation method. Specifically, as shown in FIG. 1C, a hole-injection layer 111, a hole-transport layer 112, a light-emitting layer 113, an electron-transport layer 114, and an electron-injection layer 115 are sequentially stacked as the EL layer 103 between the first electrode 101 and the second electrode 102 by a vacuum evaporation method. In each of the light-emitting devices in FIG. 1B, FIG. 1D, and FIG. 1E, when the first electrode 101 is an anode, a hole-injection layer 111a and a hole-transport layer 112a of an EL layer 103a are sequentially stacked over the first electrode 101 by a vacuum evaporation method. After the EL layer 103a and a charge-generation layer 106 are formed, a hole-injection layer 111b and a hole-transport layer 112b of the EL layer 103b are sequentially stacked over the charge-generation layer 106 in a similar manner.

### <Hole-Injection Layer>

The hole-injection layers (111, 111a, and 111b) are each a layer that injects holes from the first electrode 101 which is an anode and the charge-generation layers (106, 106a, and 106b) to the EL layers (103, 103a, and 103b) and contains an organic acceptor material and a material with a high hole-injection property.

The organic acceptor material allows holes to be generated in another organic compound whose HOMO (highest occupied molecular orbital) level is close to the LUMO (lowest unoccupied molecular orbital) level of the organic acceptor material when charge separation is caused between the organic acceptor material and the organic compound. Thus, as the organic acceptor material, a compound having an electron-withdrawing group (a halogen group or a cyano group), such as a quinodimethane derivative, a chloranil derivative, or a hexaazatriphenylene derivative, can be used. For example, it is possible to use, 7,7,8,8-tetracyano-2,3,5,6-tetrafluoroquinodimethane (abbreviation: F₄-TCNQ), 3,6-difluoro-2,5,7,7,8,8-hexacyanoquinodimethane, chloranil, 2,3,6,7,10,11-hexacyano-1,4,5,8,9,12-hexaazatriphenylene (abbreviation: HAT-CN), 1,3,4,5,7,8-hexafluorotetracyano-naphthoquinodimethane (abbreviation: F6-TCNNQ), 2-(7-dicyanomethylen-1,3,4,5,6,8,9,10-octafluoro-7H-pyren-2-ylidene)malononitrile, or the like can be used. Note that among organic acceptor materials, a compound in which electron-withdrawing groups are bonded to fused aromatic rings each having a plurality of heteroatoms, such as HAT-CN, is particularly preferred because it has a high acceptor property and stable film quality against heat. In addition, a [3]radialene derivative having an electron-withdrawing group (in particular, a cyano group, a halogen group such as a fluoro group, or the like) has a very high electron-accepting property and thus is preferable. Specific examples include α,α′,α″−1,2,3-cyclopropanetriylidenetris[4-cyano-2,3,5,6-tetrafluorobenzeneacetonitrile], α,α′,α″−1,2,3-cyclopropanetriylidenetris[2,6-dichloro-3,5-difluoro-4-(trifluoromethyl)benzeneacetonitrile], and α,α′,α″-1,2,3-cyclopropanetriylidenetris[2,3,4,5,6-pentafluorobenzeneacetonitrile].

As the material having a high hole-injection property, an oxide of a metal belonging to Group 4 to Group 8 in the periodic table (e.g., a transition metal oxide such as a molybdenum oxide, a vanadium oxide, a ruthenium oxide, a tungsten oxide, or a manganese oxide) can be used. As specific examples, molybdenum oxide, vanadium oxide, niobium oxide, tantalum oxide, chromium oxide, tungsten oxide, manganese oxide, and rhenium oxide can be given. In particular, molybdenum oxide is preferable because it is stable in the air, has a low hygroscopic property, and is easily handled. It is also possible to use a phthalocyanine-based compound such as phthalocyanine (abbreviation: H₂Pc) or copper phthalocyanine (abbreviation: CuPc), or the like.

In addition to the above materials, it is also possible to use an aromatic amine compound, which is a low molecular compound, such as 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenyl amino]biphenyl (abbreviation: DPAB), N,N′-bis{4-[bis(3-methy 1phenyl)amino]phenyl}-N,N′-diphenyl-(1,1′-biphenyl)-4,4′-diamine (abbreviation: DNTPD), 1,3,5-tris[N-(4-diphenylaminophenyl)-N-phenylamino]benzene (abbreviation: DPA3B), 3-[N-(9-phenyl carbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), or 3-[N-(1-naphthyl)-N-(9-phenylcarbazol-3-yl)amino]-9-phenylcarbazole (abbreviation: PCzPCN1).

It is also possible to use a high molecular compound (an oligomer, a dendrimer, a polymer, or the like) such as poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide] (abbreviation: PTPDMA), or poly[N,N′-bis(4-butylphenyl)-N,N′-bis(phenyl)benzidine] (abbreviation: Poly-TPD). Alternatively, it is also possible to use a high molecular compound to which acid is added, such as poly(3,4-ethylenedioxythiophene)/poly(styrenesulfonic acid) (abbreviation: PEDOT/PSS) or polyaniline/poly(styrenesulfonic acid) (PAni/PSS).

Alternatively, as the material having a high hole-injection property, a composite material containing a hole-transport material and the above-described organic acceptor material (electron-accepting material) can be used. In this case, the organic acceptor material extracts electrons from a hole-transport material, so that holes are generated in the hole-injection layer 111 and the holes are injected into the light-emitting layer 113 through the hole-transport layer 112. Note that the hole-injection layer 111 may be formed as a single layer made of a composite material containing a hole-transport material and an organic acceptor material (electron-accepting material), or may be formed by stacking a layer containing a hole-transport material and a layer containing an organic acceptor material (electron-accepting material).

The hole-transport material is preferably a substance having a hole mobility higher than or equal to 1×10⁻⁶ cm²/Vs in the case where the square root of the electric field strength [V/cm] is 600. Note that other substances can be used as long as they have a property of transporting more holes than electrons.

As the hole-transport material, materials having a high hole-transport property, such as a π-electron rich heteroaromatic compound (e.g., a carbazole derivative, a furan derivative, and a thiophene derivative) and an aromatic amine (a compound having an aromatic amine skeleton), are preferred.

Examples of the above carbazole derivative (a compound having a carbazole skeleton) include a bicarbazole derivative (e.g., a 3,3′-bicarbazole derivative) and an aromatic amine having a carbazolyl group.

Specific examples of the bicarbazole derivative (e.g., a 3,3′-bicarbazole derivative) include 3,3′-bis(9-phenyl-9H-carbazole) (abbreviation: PCCP), 9,9′-bis(1,1′-biphenyl-4-yl)-3,3′-bi-9H-carbazole (BisBPCz), 9,9′-bis(1,1′-biphenyl-3-yl)-3,3′-bi-9H-carbazole, 9-(1,1′-biphenyl-3-yl)-9′-(1,1′-biphenyl-4-yl)-9H,9′H-3,3′-bicarbazole (abbreviation: mBPCCBP), and 9-(2-naphthyl)-9′-phenyl-9H,9′H-3,3′-bicarbazole (abbreviation: (3NCCP).

Specific examples of the above aromatic amine having a carbazolyl group include 4-phenyl-4′-(9-phenyl-9H-carbazol-3-yl)triphenylamine (abbreviation: PCBA1BP), N-(4-biphenyl)-N-(9,9-dimethyl-9H-fluoren-2-yl)-9-phenyl-9H-carbazol-3-amine (abbreviation: PCBiF), N-(1,1′-biphenyl-4-yl)-N-[4-(9-phenyl-9H-carbazol-3-yl)phenyl]-9,9-dimethyl-9H-fluoren-2-amine (abbreviation: PCBBiF), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 4-phenyldiphenyl-(9-phenyl-9H-carbazol-3-yl)amine (abbreviation: PCA1BP), N,N-bis(9-phenylcarbazol-3-yl)-N,N-diphenylbenzene-1,3-diamine (abbreviation: PCA2B), N,N,N′-triphenyl-N,N,N′-tris(9-phenylcarbazol-3-yl)benzene-1,3,5-triamine (abbreviation: PCA3B), 9,9-dimethyl-N-phenyl-N-[4-(9-phenyl-9H-carbazol-3-yl)phenyl]fluoren-2-amine (abbreviation: PCBAF), N-phenyl-N-[4-(9-phenyl-9H-carbazol-3-yl)phenyl]spiro-9,9′-bifluoren-2-amine (abbreviation: PCBASF), 3-[N-(9-phenylcarbazol-3-yl)-N-phenyl amino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), 3-[N-(1-naphthyl)-N-(9-phenylcarbazol-3-yl)amino]-9-phenylcarbazole (abbreviation: PCzPCN1), 3-[N-(4-diphenylaminophenyl)-N-phenylamino]-9-phenylcarbazole (abbreviation: PCzDPA1), 3,6-bis[N-(4-diphenylaminophenyl)-N-phenylamino]-9-phenylcarbazole (abbreviation: PCzDPA2), 3,6-bis[N-(4-diphenylaminophenyl)-N-(1-naphthyl)amino]-9-phenylcarbazole (abbreviation: PCzTPN2), 2-[N-(9-phenylcarbazol-3-yl)-N-phenylamino]spiro-9,9′-bifluorene (abbreviation: PCASF), N-[4-(9H-carbazol-9-yl)phenyl]-N-(4-phenyl)phenylaniline (abbreviation: YGA1BP), N,N-bis[4-(carbazol-9-yl)phenyl]-N,N′-diphenyl-9,9-Dimethylfluorene-2,7-diamine (abbreviation: YGA2F), and 4,4′,4″-tris(carbazol-9-yl)triphenylamine (abbreviation: TCTA).

In addition to the above, other examples of the carbazole derivative include 3-[4-(9-phenanthryl)-phenyl]-9-phenyl-9H-carbazole (abbreviation: PCPPn), 3-[4-(1-naphthyl)-phenyl]-9-phenyl-9H-carbazole (abbreviation: PCPN), 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), 1,3,5-tris[4-(N-carbazolyl)phenyl]benzene (abbreviation: TCPB), and 9-[4-(10-phenyl-9-anthracenyl)phenyl]-9H-carbazole (abbreviation: CzPA).

Specific examples of the furan derivative (the compound having a furan skeleton) include 4,4′,4″-(benzene-1,3,5-triyl)tri(dibenzofuran) (abbreviation: DBF3P-II), and 4-{3-[3-(9-phenyl-9H-fluoren-9-yl)phenyl]phenyl}dibenzofuran (abbreviation: mmDBFFLBi-II).

Specific examples of the thiophene derivative (the compound having a thiophene skeleton) include compounds having a thiophene skeleton), such as 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), and 4-[4-(9-phenyl-9H-fluoren-9-yl)phenyl]-6-phenyldibenzothiophene (abbreviation: DBTFLP-IV).

Specific examples of the aromatic amine include 4,4′-bis[N-(1-naphthyl)-N-phenylamino]biphenyl (abbreviation: NPB or α-NPD), N,N-bis(3-methylphenyl)-N,N-diphenyl-[1,1′-biphenyl]-4,4′-diamine (abbreviation: TPD), 4,4′-bis[N-(spiro-9,9′-bifluoren-2-yl)-N-phenylamino]biphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), N-(9,9-dimethyl-9H-fluoren-2-yl)-N-{9,9-dimethyl-2-[N-phenyl-N′-(9,9-dimethyl-9H-fluoren-2-yl)amino]-9H-fluoren-7-yl}phenylamine (abbreviation: DFLADFL), N-(9,9-dimethyl-2-diphenylamino-9H-fluoren-7-yl)diphenylamine (abbreviation: DPNF), 2-[N-(4-diphenylaminophenyl)-N-phenylamino]spiro-9,9′-bifluorene (abbreviation: DPASF), 2,7-bis[N-(4-diphenylaminophenyl)-N-phenylamino]-spiro-9,9′-bifluorene (abbreviation: DPA2SF), 4,4′,4″-tris[N-(1-naphthyl)-N-phenylamino]triphenylamine (abbreviation: 1′-TNATA), 4,4′,4″-tris(N,N-diphenylamino)triphenylamine (abbreviation: TDATA), 4,4′,4″-tris[N-(3-methylphenyl)-N-phenylamino]triphenylamine (abbreviation: m-MTDATA), N,N-di(p-tolyl)-N,N-diphenyl-p-phenylenediamine (abbreviation: DTDPPA), 4,4′-bis[N-(4-diphenylaminophenyl)-N-phenylamino]biphenyl (abbreviation: DPAB), DNTPD, 1,3,5-tris[N-(4-diphenylaminophenyl)-N-phenyl amino]benzene (abbreviation: DPA3B), N-(4-biphenyl)-6,N-diphenylbenzo[b]naphtho[1,2-d]furan-8-amine (abbreviation: BnfABP), N,N-bis(4-biphenyl)-6-phenylbenzo[b]naphtho[1,2-d]furan-8-amine (abbreviation: BBABnf), 4,4′-bis(6-phenylbenzo[b]naphtho[1,2-d]furan-8-yl)-4″-phenyltriphenylamine (abbreviation: BnfBB1BP), N,N-bis(4-biphenyl)benzo[b]naphtho[1,2-d]furan-6-amine (abbreviation: BBABnf(6)), N,N-bis(4-biphenyl)benzo[b]naphtho[1,2-d]furan-8-amine (abbreviation: BBABnf(8)), N,N-bis(4-biphenyl)benzo[b]naphtho[2,3-d]furan-4-amine (abbreviation: BBABnf(II)(4)), N,N-bis[4-(dibenzofuran-4-yl)phenyl]-4-amino-p-terphenyl (abbreviation: DBfBB1TP), N-[4-(dibenzothiophen-4-yl)phenyl]-N-phenyl-4-biphenylamine (abbreviation: ThBA1BP), 4-(2-naphthyl)-4′,4″-diphenyltriphenylamine (abbreviation: BBAβNB), 4-[4-(2-naphthyl)phenyl]-4′,4″-diphenyltriphenylamine (abbreviation: BBAβNBi), 4,4′-diphenyl-4″-(6;1′-binaphthyl-2-yl)triphenylamine (abbreviation: BBAαNβNB), 4,4′-diphenyl-4″-(7;1′-binaphthyl-2-yl)triphenylamine (abbreviation: BBAαNβNB-03), 4,4′-diphenyl-4″-(7-phenyl)naphthyl-2-yltriphenylamine (abbreviation: BBAPβNB-03), 4,4′-diphenyl-4″-(6;2′-binaphthyl-2-yl)triphenylamine (abbreviation: BBA(βN2)B), 4,4′-diphenyl-4″-(7;2′-binaphthyl-2-yl)triphenylamine (abbreviation: BBA(βN2)B-03), 4,4′-diphenyl-4″-(4;2′-binaphthyl-1-yl)triphenylamine (abbreviation: BBAβNαNB), 4,4′-diphenyl-4″-(5;2′-binaphthyl-1-yl)triphenylamine (abbreviation: BBAβNαNB-02), 4-(4-biphenylyl)-4′-(2-naphthyl)-4″-phenyltriphenylamine (abbreviation: TPBiAβNB), 4-(3-biphenylyl)-4′-[4-(2-naphthyl)phenyl]-4″-phenyltriphenylamine (abbreviation: mTPBiAβNBi), 4-(4-biphenylyl)-4′-[4-(2-naphthyl)phenyl]-4″-phenyltriphenylamine (abbreviation: TPBiAβNBi), 4-phenyl-4′-(1-naphthyl)-triphenylamine (abbreviation: αNBA1BP), 4,4′-bis(1-naphthyl)triphenylamine (abbreviation: αNBB1BP), 4,4′-diphenyl-4″-[4′-(carbazol-9-yl)biphenyl-4-yl]triphenylamine (abbreviation: YGTBi1BP), 4′-[4-(3-phenyl-9H-carbazol-9-yl)phenyl]tris(1,1′-biphenyl-4-yl)amine (abbreviation: YGTBi1BP-02), 4-[4′-(carbazol-9-yl)biphenyl-4-yl]-4′-(2-naphthyl)-4″-phenyltriphenylamine (abbreviation: YGTBiβNB), N-[4-(9-phenyl-9H-carbazol-3-yl)phenyl]-N-[4-(1-naphthyl)phenyl]-9,9′-spirobi[9H-fluoren]-2-amine (abbreviation: PCBNBSF), N,N-bis([1,1′-biphenyl]-4-yl)-9,9′-spirobi[9H-fluoren]-2-amine (abbreviation: BBASF), N,N-bis([1,1′-biphenyl]-4-yl)-9,9′-spirobi[9H-fluoren]-4-amine (abbreviation: BBASF(4)), N-(1,1′-biphenyl-2-yl)-N-(9,9-dimethyl-9H-fluoren-2-yl)-9,9′-spirobi[9H-fluoren]-4-amine (abbreviation: oFBiSF), N-(4-biphenyl)-N-(9,9-dimethyl-9H-fluoren-2-yl)dibenzofuran-4-amine (abbreviation: FrBiF), N-[4-(1-naphthyl)phenyl]-N-[3-(6-phenyldibenzofuran-4-yl)phenyl]-1-naphthylamine (abbreviation: mPDBfBNBN), 4-phenyl-4′-[4-(9-phenylfluoren-9-yl)phenyl]triphenylamine (abbreviation: BPAFLBi), N,N-bis(9,9-dimethyl-9H-fluoren-2-yl)-9,9′-spirobi-9H-fluoren-4-amine, N,N-bis(9,9-dimethyl-9H-fluoren-2-yl)-9,9′-spirobi-9H-fluoren-3-amine, N,N-bis(9,9-dimethyl-9H-fluoren-2-yl)-9,9′-spirobi-9H-fluoren-2-amine, and N,N-bis(9,9-dimethyl-9H-fluoren-2-yl)-9,9′-spirobi-9H-fluoren-1-amine.

It is also possible to use a high molecular compound (an oligomer, a dendrimer, a polymer, or the like) such as poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide] (abbreviation: PTPDMA), or poly[N,N-bis(4-butylphenyl)-N,N-bis(phenyl)benzidine] (abbreviation: Poly-TPD) as a hole-transport material. Alternatively, it is also possible to use a high molecular compound to which acid is added, such as poly(3,4-ethylenedioxythiophene)/poly(styrenesulfonic acid) (abbreviation: PEDOT/PSS) or polyaniline/poly(styrenesulfonic acid) (PAni/PSS).

Note that the hole-transport material is not limited to the above, and one or a combination of various known materials may be used as the hole-transport material.

Note that the hole-injection layers (111, 111a, and 111b) can be formed by any of various known deposition methods, and can be formed by a vacuum evaporation method, for example.

### <Hole-Transport Layer>

The hole-transport layers (112, 112a, and 112b) are each a layer that transports the holes, which are injected from the first electrode 101 by the hole-injection layers (111, 111a, and 111b), to the light-emitting layers (113, 113a, and 113b). Note that the hole-transport layers (112, 112a, and 112b) are each a layer containing a hole-transport material. Thus, for the hole-transport layers (112, 112a, and 112b), a hole-transport material that can be used for the hole-injection layers (111, 111a, and 111b) can be used.

Note that in the light-emitting device of one embodiment of the present invention, the organic compound used for the hole-transport layers (112, 112a, and 112b) can also be used for the light-emitting layers (113, 113a, and 113b). The use of the same organic compound for the hole-transport layers (112, 112a, and 112b) and the light-emitting layers (113, 113a, and 113b) is preferable, in which case holes can be efficiently transported from the hole-transport layers (112, 112a, and 112b) to the light-emitting layers (113, 113a, and 113b).

### <Light-Emitting Layer>

The light-emitting layers (113, 113a, and 113b) each contain a light-emitting substance. The organic compound of one embodiment of the present invention is preferably used in the light-emitting layers (113, 113a, and 113b). For the light-emitting substance that can be used for the light-emitting layers (113, 113a, and 113b), it is possible to use a substance that exhibits emission color of blue, purple, bluish purple, green, yellowish green, yellow, orange, red, or the like can be used as appropriate. When different light-emitting substances are used for a plurality of light-emitting layers, different emission colors can be exhibited (for example, complementary emission colors are combined to obtain white light emission). Furthermore, a stacked-layer structure in which one light-emitting layer contains different light-emitting substances may be employed.

The light-emitting layers (113, 113a, and 113b) may each contain one or more kinds of organic compounds (a host material and the like) in addition to a light-emitting substance (a guest material).

In the case where a plurality of host materials are used in the light-emitting layers (113, 113a, and 113b), a second host material that is additionally used is preferably a substance having a larger energy gap than those of a known guest material and a first host material. Preferably, the lowest singlet excitation energy level (S1 level) of the second host material is higher than that of the first host material, and the lowest triplet excitation energy level (T1 level) of the second host material is higher than that of the guest material. Furthermore, the lowest triplet excitation energy level (T1 level) of the second host material is preferably higher than the Ti level of the first host material. With such a structure, an exciplex can be formed by the two kinds of host materials. In order to form an exciplex efficiently, it is particularly preferable to combine a compound that easily accepts holes (a hole-transport material) and a compound that easily accepts electrons (an electron-transport material). With the structure, high efficiency, low voltage, and a long lifetime can be achieved at the same time.

As an organic compound used as the host material (including the first host material and the second host material), organic compounds such as the hole-transport materials usable for the hole-transport layers (112, 112a, and 112b) described above and electron-transport materials usable for electron-transport layers (114, 114a, and 114b) described later can be used as long as they satisfy requirements for the host material used in the light-emitting layer. Another example is an exciplex formed by two or more kinds of organic compounds (the first host material and the second host material). An exciplex (also referred to as Exciplex) whose excited state is formed by a plurality of kinds of organic compounds has an extremely small difference between the S1 level and the T1 level and functions as a TADF material that can convert triplet excitation energy into singlet excitation energy. As a combination of the plurality of kinds of organic compounds forming an exciplex, for example, it is preferable that one have a t-electron deficient heteroaromatic ring and the other have a t-electron rich heteroaromatic ring. A phosphorescent substance such as an iridium-, rhodium-, or platinum-based organometallic complex or a metal complex may be used as one of the combination forming an exciplex.

The light-emitting substance that can be used for the light-emitting layers (113, 113a, and 113b) is not particularly limited, and a light-emitting substance that converts singlet excitation energy into light emission in a visible light region or a light-emitting substance that converts triplet excitation energy into light emission in a visible light region can be used.

<<Light-Emitting Substance that Converts Singlet Excitation Energy into Light>>

Other than the organic compound of one embodiment of the present invention, the following substances emitting fluorescence (fluorescent substances) can be given as the light-emitting substance that can be used for the light-emitting layer 113 and convert singlet excitation energy into light emission. Examples include a pyrene derivative, an anthracene derivative, a triphenylene derivative, a fluorene derivative, a carbazole derivative, a dibenzothiophene derivative, a dibenzofuran derivative, a dibenzoquinoxaline derivative, a quinoxaline derivative, a pyridine derivative, a pyrimidine derivative, a phenanthrene derivative, and a naphthalene derivative. A pyrene derivative is particularly preferable because it has a high emission quantum yield. Specific examples of pyrene derivatives include N,N-bis(3-methylphenyl)-N,N-bis[3-(9-phenyl-9H-fluoren-9-yl)phenyl]pyrene-1,6-diamine (abbreviation: 1,6mMemFLPAPrn), N,N′-diphenyl-N,N′-bis[4-(9-phenyl-9H-fluoren-9-yl)phenyl]pyrene-1,6-diamine (abbreviation: 1,6FLPAPrn), N,N-bis(dibenzofuran-2-yl)-N,N-diphenylpyrene-1,6-diamine (abbreviation: 1,6FrAPrn), N,N-bis(dibenzothiophen-2-yl)-N,N-diphenylpyrene-1,6-diamine (abbreviation: 1,6ThAPrn), N,N-(pyrene-1,6-diyl)bis[(N-phenylbenzo[b]naphtho[1,2-d]furan)-6-amine] (abbreviation: 1,6BnfAPrn), N,N-(pyrene-1,6-diyl)bis[(N-phenylbenzo[b]naphtho[1,2-d]furan)-8-amine] (abbreviation: 1,6BnfAPrn-02), and N,N-(pyrene-1,6-diyl)bis[(6,N-diphenylbenzo[b]naphtho[1,2-d]furan)-8-amine] (abbreviation: 1,6BnfAPrn-03).

In addition, it is possible to use 5,6-bis[4-(10-phenyl-9-anthryl)phenyl]-2,2′-bipyridine (abbreviation: PAP2BPy), 5,6-bis[4′-(10-phenyl-9-anthryl)biphenyl-4-yl]-2,2′-bipyridine (abbreviation: PAPP2BPy), N,N-bis[4-(9H-carbazol-9-yl)phenyl]-N,N-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), 4-(10-phenyl-9-anthryl)-4′-(9-phenyl-9H-carbazol-3-yl)triphenylamine (abbreviation: PCBAPA), 4-[4-(10-phenyl-9-anthryl)phenyl]-4′-(9-phenyl-9H-carbazol-3-yl)tri phenyl amine (abbreviation: PCBAPBA), perylene, 2,5,8,11-tetra-tert-butylperylene (abbreviation: TBP), N,N′-(2-tert-butyl anthracene-9,10-diyldi-4,1-phenylene)bis[N,N,N-triphenyl-1,4-phenylenediamine] (abbreviation: DPABPA), N,9-diphenyl-N-[4-(9,10-diphenyl-2-anthryl)phenyl]-9H-carbazol-3-amine (abbreviation: 2PCAPPA), N-[4-(9,10-diphenyl-2-anthryl)phenyl]-N,N′,N′-triphenyl-1,4-phenylenedi amine (abbreviation: 2DPAPPA), or the like.

It is also possible to use, for example, N-[9,10-bis(1,1′-biphenyl-2-yl)-2-anthryl]-N,9-diphenyl-9H-carbazol-3-amine (abbreviation: 2PCABPhA), N-(9,10-diphenyl-2-anthryl)-N,N,N-triphenyl-1,4-phenylenediamine (abbreviation: 2DPAPA), N-[9,10-bis(1,1′-biphenyl-2-yl)-2-anthryl]-N,N,N-tri phenyl-1,4-phenylenediamine (abbreviation: 2DPABPhA), 9,10-bis(1,1′-biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), coumarin 545T, N,N′-diphenylquinacridone (abbreviation: DPQd), rubrene, 5,12-bis(1,1′-biphenyl-4-yl)-6,11-diphenyltetracene (abbreviation: BPT), 2-(2-{2-[4-(dimethylamino)phenyl]ethenyl}-6-methyl-4H-pyran-4-ylidene)propanedinitrile (abbreviation: DCM1), 2-{2-methyl-6-[2-(2,3,6,7-tetrahydro-1H,5H-benzo[i]quinolizin-9-yl)ethenyl]-4H-pyran-4-ylidene}propanedinitrile (abbreviation: DCM2), N,N,N′,N′-tetrakis(4-methylphenyl)tetracene-5,11-diamine (abbreviation: p-mPhTD), 7,14-diphenyl-N,N,N,N-tetrakis(4-methylphenyl)acenaphtho[1,2-a]fluoranthene-3,10-diamine (abbreviation: p-mPhAFD), 2-{2-isopropyl-6-[2-(1,1,7,7-tetramethyl-2,3,6,7-tetrahydro-1H,5H-benzo quinolizin-9-yl)ethenyl]-4H-pyran-4-ylidene}propanedinitrile (abbreviation: DCJT1), 2-{2-tert-butyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTB), 2-(2,6-bis{2-[4-(dimethylamino)phenyl]ethenyl}-4H-pyran-4-ylidene)propanedinitrile (abbreviation: BisDCM), 2-{2,6-bis[2-(8-methoxy-1,1,7,7-tetramethyl-2,3,6,7-tetrahydro-1H,5H-benzo quinolizin-9-yl)ethenyl]-4H-pyran-4-ylidene}propanedinitrile (abbreviation: BisDCJTM), 1,6BnfAPrn-03,3,10-bis[N-(9-phenyl-9H-carbazol-2-yl)-N-phenyl amino]naphtho[2,3-b;6,7-b′]bisbenzofuran (abbreviation: 3,10PCA2Nbf(IV)-02), or 3,10-bis[N-(dibenzofuran-3-yl)-N-phenylamino]naphtho[2,3-b;6,7-b′]bisbenzofuran (abbreviation: 3,10FrA2Nbf(IV)-02). In particular, pyrenediamine compounds such as 1,6FLPAPrn, 1,6mMemFLPAPrn, and 1,6BnfAPrn-03 can be used, for example.

<<Light-Emitting Substance that Converts Triplet Excitation Energy into Light>>

Next, as an example of the light-emitting substance that converts triplet excitation energy into light, a substance that emits phosphorescence (a phosphorescent substance) and a thermally activated delayed fluorescent (TADF) material that exhibits thermally activated delayed fluorescence can be given as a substance that can be used for the light-emitting layer 113.

A phosphorescent substance refers to a compound that exhibits phosphorescence but does not exhibit fluorescence at a temperature higher than or equal to low temperatures (e.g., 77 K) and lower than or equal to room temperature (i.e., higher than or equal to 77 K and lower than or equal to 313 K). The phosphorescent substance preferably contains a metal element with large spin-orbit interaction, and can be an organometallic complex, a metal complex (platinum complex), a rare earth metal complex, or the like. Specifically, a transition metal element is preferable and it is particularly preferable that a platinum group element (ruthenium (Ru), rhodium (Rh), palladium (Pd), osmium (Os), iridium (Ir), or platinum (Pt)), especially iridium, be contained, in which case the transition probability relating to direct transition between the singlet ground state and the triplet excited state can be increased.

<<Phosphorescent Substance (from 450 nm to 570 nm: Blue or Green)>>

As a phosphorescent substance that emits blue or green light and whose emission spectrum has a peak wavelength greater than or equal to 450 nm and less than or equal to 570 nm, the following substances can be given.

The examples include organometallic complexes having a 4H-triazole skeleton, such as tris{2-[5-(2-methylphenyl)-4-(2,6-dimethylphenyl)-4H-1,2,4-triazol-3-yl-κN²]phenyl-κC}iridium(III) (abbreviation: [Ir(mpptz-dmp)₃]), tris(5-methyl-3,4-diphenyl-4H-1,2,4-triazolato) iridium(III) (abbreviation: [Ir(Mptz)₃]), tris[4-(3-biphenyl)-5-isopropyl-3-phenyl-4H-1,2,4-triazolato]iridium(III) (abbreviation: [Ir(iPrptz-3b)₃]), and tris[3-(5-biphenyl)-5-isopropyl-4-phenyl-4H-1,2,4-triazolato]iridium(III) (abbreviation: [Ir(iPr5btz)₃]); organometallic complexes having a 1H-triazole skeleton, such as tris[3-methyl-1-(2-methylphenyl)-5-phenyl-1H-1,2,4-triazolato]iridium(III) (abbreviation: [Ir(Mptz1-mp)₃]) and tris(1-methyl-5-phenyl-3-propyl-1H-1,2,4-triazolato)iridium(III) (abbreviation: [Ir(Prptz1-Me)₃]); organometallic complexes having an imidazole skeleton, such as fac-tris[1-(2,6-diisopropylphenyl)-2-phenyl-1H-imidazole]iridium(III) (abbreviation: [Ir(iPrpmi)₃]) and tris[3-(2,6-dimethylphenyl)-7-methylimidazo[1,2-f]phenanthridinato]iridium(III) (abbreviation: [Ir(dmpimpt-Me)₃]); and organometallic complexes in which a phenylpyridine derivative having an electron-withdrawing group is a ligand, such as bis[2-(4′,6′-difluorophenyl) pyridinato-N,C^{2′}]iridium(III) tetrakis(1-pyrazolyl) borate (abbreviation: FIr6), bis[2-(4′,6′-difluorophenyl)pyridinato-N,C^{2′}]iridium(III) picolinate (abbreviation: FIrpic), bis{2-[3′,5′-bis(trifluoromethyl) phenyl]pyridinato-N,C^{2′}}iridium(III) picolinate (abbreviation: [Ir(CF₃ppy)₂(pic)]), and bis[2-(4′,6′-difluorophenyl) pyridinato-N,C^{2′}]iridium(III) acetylacetonate (abbreviation: FIr(acac)).

<<Phosphorescent Substance (from 495 nm to 590 nm: Green or Yellow)>>

As a phosphorescent substance that exhibits green or yellow and whose emission spectrum has a peak wavelength greater than or equal to 495 nm and less than or equal to 590 nm, the following substances can be given.

For example, organometallic iridium complexes having a pyrimidine skeleton, such as tris(4-methyl-6-phenylpyrimidinato)iridium(III) (abbreviation: [Ir(mppm)₃]), tris(4-t-butyl-6-phenylpyrimidinato)iridium(III) (abbreviation: [Ir(tBuppm)₃]), (acetylacetonato)bis(6-methyl-4-phenylpyrimidinato)iridium(III) (abbreviation: [Ir(mppm)₂(acac)]), (acetylacetonato)bis(6-tert-butyl-4-phenylpyrimidinato)iridium(III) (abbreviation: [Ir(tBuppm)₂(acac)]), (acetyl acetonato)bis[6-(2-norbornyl)-4-phenylpyrimidinato]iridium(III) (abbreviation: [Ir(nbppm)₂(acac)]), (acetyl acetonato)bis[5-methyl-6-(2-methylphenyl)-4-phenylpyrimidinato]iridium(III) (abbreviation: [Ir(mpmppm)₂(acac)]), (acetyl acetonato)bi s {4,6-dimethyl-2-[6-(2,6-dimethylphenyl)-4-pyrimidinyl-κN3]phenyl-κC}iridium(III) (abbreviation: [Ir(dmppm-dmp)₂(acac)]), and (acetylacetonato)bi s (4,6-diphenylpyrimidinato)iridium(III) (abbreviation: [Ir(dppm)₂(acac)]); organometallic iridium complexes having a pyrazine skeleton, such as (acetylacetonato)bis(3,5-dimethyl-2-phenylpyrazinato)iridium(III) (abbreviation: [Ir(mppr-Me)₂(acac)]) and (acetyl acetonato)bis(5-isopropyl-3-methyl-2-phenylpyrazinato)iridium(III) (abbreviation: [Ir(mppr-iPr)₂(acac)]); organometallic iridium complexes having a pyridine skeleton, such as (acetylacetonato)bis(3,5-dimethyl-2-phenylpyrazinato)iridium(III) (abbreviation: [Ir(mppr-Me)₂(acac)]) and (acetylacetonato)bis(5-isopropyl-3-methyl-2-phenylpyrazinato)iridium(III) (abbreviation: [Ir(mppr-iPr)₂(acac)]); organometallic iridium complexes having a pyridine skeleton, such as tris(2-phenylpyridinato-N,C^{2′})iridium(III) (abbreviation: [Ir(ppy)₃]), bis(2-phenylpyridinato-N,C^{2′})iridium(III) acetylacetonate (abbreviation: [Ir(ppy)₂(acac)]), bis(benzo[h]quinolinato)iridium(III) acetyl acetonate (abbreviation: [Ir(bzq)₂(acac)]), tris(benzo[h]quinolinato)iridium(III) (abbreviation: [Ir(bzq)₃]), tris(2-phenylquinolinato-N,C^{2′})iridium(III) (abbreviation: [Ir(pq)₃]), bis(2-phenylquinolinato-N,C^{2′})iridium(III) acetylacetonate (abbreviation: [Ir(pq)₂(acac)]), bis[2-(2-pyridinyl-κN)phenyl-κC][2-(4-phenyl-2-pyridinyl-κN) phenyl-κC]iridium(III) (abbreviation: [Ir(ppy)₂(4dppy)]), and bis[2-(2-pyridinyl-κN)phenyl-κC][2-(4-methyl-5-phenyl-2-pyridinyl-κN) phenyl-KC]; organometallic complexes such as bis(2,4-diphenyl-1,3-oxazolato-N,C^{2′})iridium(III) acetylacetonate (abbreviation: [Ir(dpo)₂(acac)]), bis{2-[4′-(perfluorophenyl)phenyl]pyridinato-N,C^{2′}}iridium(III) acetylacetonate (abbreviation: [Ir(p-PF-ph)₂(acac)]), and bis(2-phenylbenzothiazolato-N,C^{2′})iridium(III) acetylacetonate (abbreviation: [Ir(bt)₂(acac)]); and rare earth metal complexes such as tris(acetylacetonato) (monophenanthroline)terbium(III) (abbreviation: [Tb(acac)₃(Phen)]) can be given.

<<Phosphorescent Substance (from 570 nm to 750 nm: Yellow or Red)>>

As a phosphorescent substance that exhibits yellow or red and whose emission spectrum has a peak wavelength greater than or equal to 570 nm and less than or equal to 750 nm, the following substances can be given.

For example, organometallic complexes having a pyrimidine skeleton, such as (diisobutyrylmethanato)bis[4,6-bis(3-methylphenyl)pyrimidinato]iridium(III) (abbreviation: [Ir(5mdppm)₂(dibm)]), bis[4,6-bis(3-methylphenyl)pyrimidinato](dipivaloylmethanato)iridium(III) (abbreviation: [Ir(5mdppm)₂(dpm)]), and (dipivaloylmethanato)bis[4,6-di(naphthalen-1-yl)pyrimidinato]iridium(III) (abbreviation: [Ir(d1npm)₂(dpm)]); organometallic complexes having a pyrazine skeleton, such as (acetylacetonato)bis(2,3,5-triphenylpyrazinato)iridium(III) (abbreviation: [Ir(tppr)₂(acac)]), bis(2,3,5-triphenylpyrazinato)(dipivaloylmethanato)iridium(III) (abbreviation: [Ir(tppr)₂(dpm)]), bis {4,6-dimethyl-2-[3-(3,5-dimethylphenyl)-5-phenyl-2-pyrazinyl-κN]phenyl-κC}(2,6-dimethyl-3,5-heptanedionato-κ²O,O′)iridium(III) (abbreviation: [Ir(dmdppr-P)₂(dibm)]), bis{4,6-dimethyl-2-[5-(4-cyano-2,6-dimethylphenyl)-3-(3,5-dimethylphenyl)-2-pyrazinyl-_{κ}N]phenyl-_{κ}C}(2,2,6,6-tetramethyl-3,5-heptanedionato-κ²O,O′)iridium(III) (abbreviation: [Ir(dmdppr-dmCP)₂(dpm)]), bis[2-[5-(2,6-dimethylphenyl)-3-(3,5-dimethylphenyl)-2-pyrazinyl-_{κ}N]-4,6-dimethylphenyl-_{κ}C](2,2′,6,6′-tetramethyl-3,5-heptadionato-_{κ}2O,O′)iridium(III) (abbreviation: [Ir(dmdppr-dmp)₂(dpm)]), (acetylacetonato)bis[2-methyl-3-phenylquinoxalinato-N,C^{2′}]iridium(III) (abbreviation: [Ir(mpq)₂(acac)]), (acetylacetonato)bis(2,3-diphenylquinoxalinato-N,C^{2′})iridium(III) (abbreviation: [Ir(dpq)₂(acac)]), and (acetylacetonato)bis[2,3-bis(4-fluorophenyl)quinoxalinato]iridium(III) (abbreviation: [Ir(Fdpq)₂(acac)]); organometallic complexes having a pyridine skeleton, such as tris(1-phenylisoquinolinato-N,C^{2′})iridium(III) (abbreviation: [Ir(piq)₃]), bis(1-phenylisoquinolinato-N,C^{2′})iridium(III) acetylacetonate (abbreviation: [Ir(piq)₂(acac)]), and bis[4,6-dimethyl-2-(2-quinolinyl-_{κ}N)phenyl-_{κ}C](2,4-pentanedionato-_{κ}²O,O′)iridium(III) (abbreviation: [Ir(dmpqn)₂(acac)]); platinum complexes such as 2,3,7,8,12,13,17,18-octaethyl-21H,23H-porphyrinplatinum(II) (abbreviation: [PtOEP]); and rare earth metal complexes such as tris(1,3-diphenyl-1,3-propanedionato)(monophenanthroline)europium(III) (abbreviation: [Eu(DBM)₃(Phen)]) and tris[1-(2-thenoyl)-3,3,3-trifluoroacetonato](monophenanthroline)europium(III) (abbreviation: [Eu(TTA)₃(Phen)]) can be given.

### <<TADF Material>>

Any of materials shown below can be used as the TADF material. The TADF material refers to a material that has a small difference (preferably, less than or equal to 0.2 eV) between the S1 level and the T1 level, can up-convert triplet excited state into singlet excited state (reverse intersystem crossing) using a little thermal energy, and efficiently exhibits light emission (fluorescence) from the singlet excited state. The thermally activated delayed fluorescence is efficiently obtained under the condition where the difference in energy between the triplet excited energy level and the singlet excited energy level is greater than or equal to 0 eV and less than or equal to 0.2 eV, preferably greater than or equal to 0 eV and less than or equal to 0.1 eV. Note that delayed fluorescence by the TADF material refers to light emission having a spectrum similar to that of normal fluorescence and an extremely long lifetime. The lifetime is 1×10⁻⁶ seconds or longer, preferably 1×10⁻³ seconds or longer.

Examples of the TADF material include fullerene, a derivative thereof, an acridine derivative such as proflavine, and eosin. Other examples include a metal-containing porphyrin such as a porphyrin containing magnesium (Mg), zinc (Zn), cadmium (Cd), tin (Sn), platinum (Pt), indium (In), or palladium (Pd). Examples of the metal-containing porphyrin include a protoporphyrin-tin fluoride complex (abbreviation: SnF₂(Proto IX)), a mesoporphyrin-tin fluoride complex (abbreviation: SnF₂(Meso IX)), a hematoporphyrin-tin fluoride complex (abbreviation: SnF₂(Hemato IX)), a coproporphyrin tetramethyl ester-tin fluoride complex (abbreviation: SnF₂(Copro III-4Me)), an octaethylporphyrin-tin fluoride complex (abbreviation: SnF₂(OEP)), an etioporphyrin-tin fluoride complex (abbreviation: SnF₂ (Etio I)), and an octaethylporphyrin-platinum chloride complex (abbreviation: PtCl₂OEP).

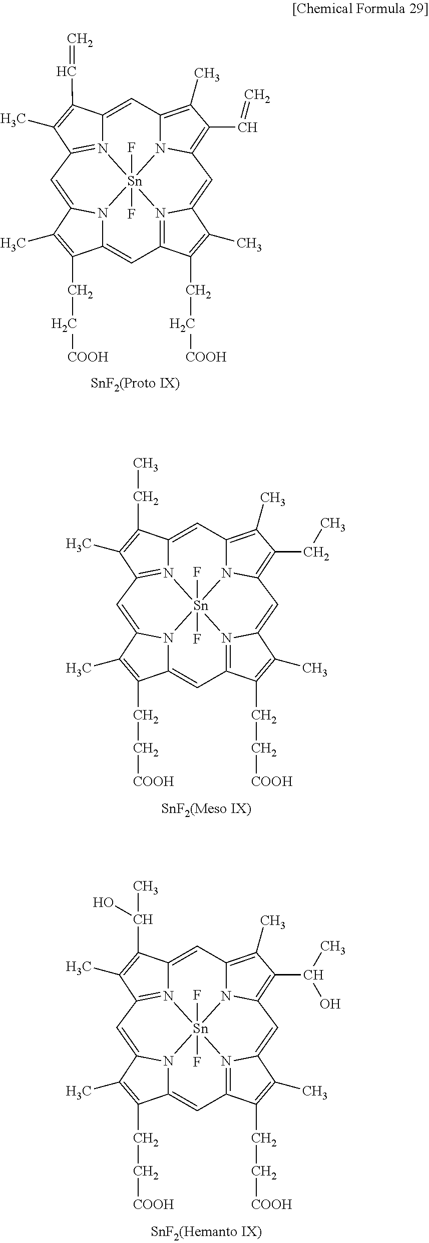

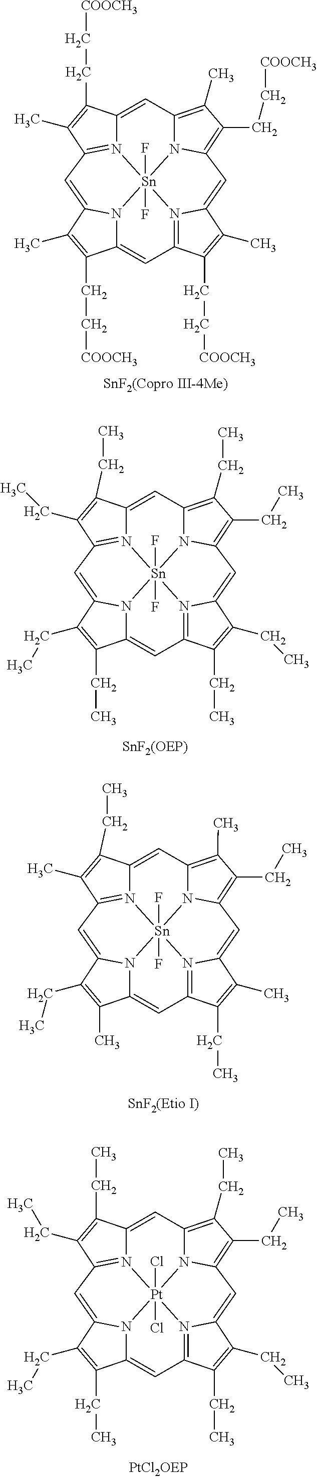

Alternatively, a heterocyclic compound having a n-electron rich heteroaromatic ring and a π-electron deficient heteroaromatic ring, such as 2-(biphenyl-4-yl)-4,6-bis(12-phenylindolo[2,3-a]carbazol-11-yl)-1,3,5-triazine (abbreviation: PIC-TRZ), 2-{4-[3-(N-phenyl-9H-carbazol-3-yl)-9H-carbazol-9-yl]phenyl}-4,6-diphenyl-1,3,5-triazine (abbreviation: PCCzPTzn), 2-[4-(10H-phenoxazin-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4-(9,9-dimethyl-9,10-dihydroacridine)phenyl]sulfone (abbreviation: DMAC-DP S), 10-phenyl-10H,10′H-spiro[acridin-9,9′-anthracen]-10′-one (abbreviation: ACRSA), 4-(9′-phenyl-3,3′-bi-9H-carbazol-9-yl)benzofuro[3,2-d]pyrimidine (abbreviation: 4PCCzBfpm), 4-[4-(9′-phenyl-3,3′-bi-9H-carbazol-9-yl)phenyl]benzofuro[3,2-d]pyrimidine (abbreviation: 4PCCzPBfpm), and 9-[3-(4,6-diphenyl-1,3,5-triazin-2-yl)phenyl]-9′-phenyl-2,3′-bi-9H-carbazole (abbreviation: mPCCzPTzn-02), may be used.

Note that a substance in which a π-electron rich heteroaromatic ring is directly bonded to a π-electron deficient heteroaromatic ring is particularly preferable because both the donor property of the π-electron rich heteroaromatic ring and the acceptor property of the π-electron deficient heteroaromatic ring are improved and the energy difference between the singlet excited state and the triplet excited state becomes small.

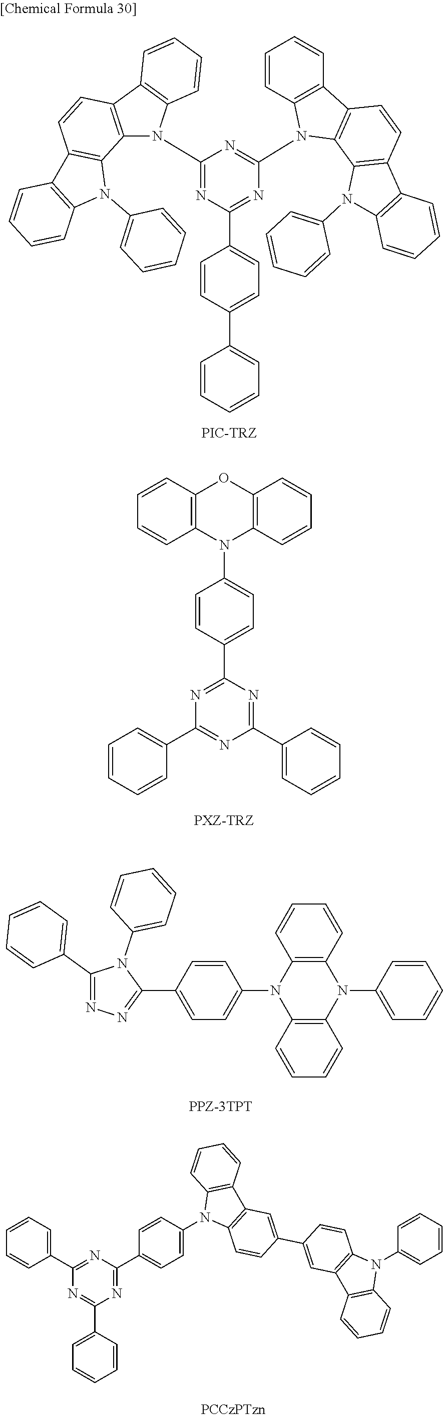 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-c3ccc(N4c5ccccc5Oc5ccccc54)cc3)n2)cc1 ; c1ccc(-c2nnc(-c3ccc(N4c5ccccc5N(c5ccccc5)c5ccccc54)cc3)n2-c2ccccc2)cc1 ; c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1]

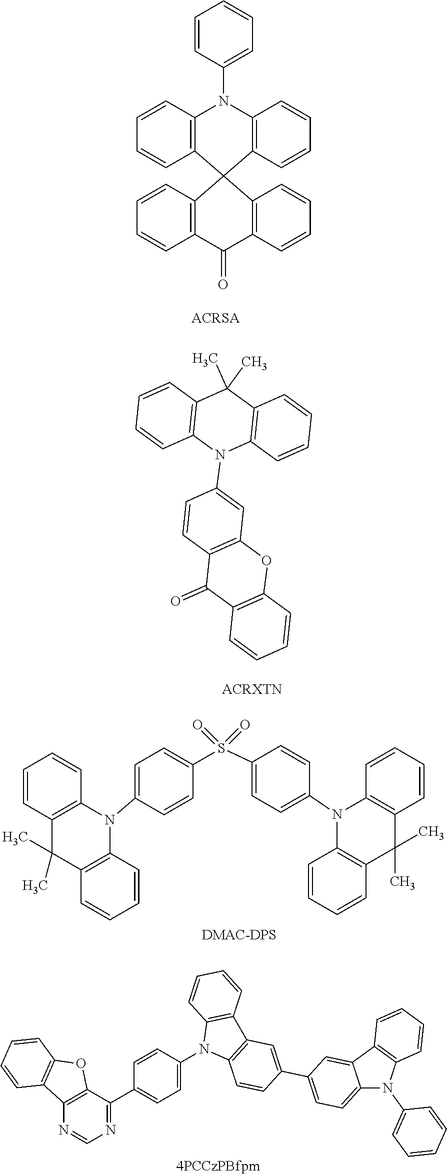 [SMILES: O=C1c2ccccc2C2(c3ccccc31)c1ccccc1N(c1ccccc1)c1ccccc12 ; CC1(C)c2ccccc2N(c2ccc3c(=O)c4ccccc4oc3c2)c2ccccc21 ; CC1(C)c2ccccc2N(c2ccc(S(=O)(=O)c3ccc(N4c5ccccc5C(C)(C)c5ccccc54)cc3)cc2)c2ccccc21 ; c1ccc(-n2c3ccccc3c3cc(-c4ccc5c(c4)c4ccccc4n5-c4ccc(-c5ncnc6c5oc5ccccc56)cc4)ccc32)cc1]

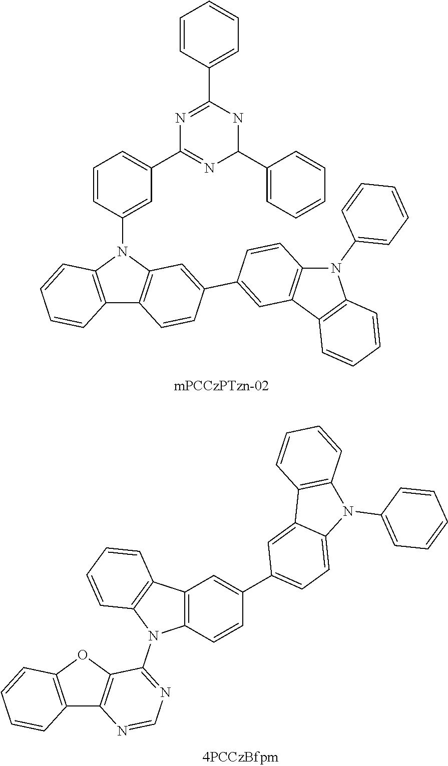

In addition to the above, as the material that has a function of converting triplet excitation energy into light emission, a nanostructure of a transition metal compound having a perovskite structure is also given. In particular, a nanostructure of a metal-halide perovskite material is preferable. The nanostructure is preferably a nanoparticle or a nanorod.

As the organic compounds (the host material and the like) used in combination with any of the above light-emitting substances (guest material) in the light-emitting layers (113, 113a, 113b, and 113c), one or more kinds of substances having a larger energy gap than the light-emitting substance (the guest material) are selected to be used.

### <<Host Material for Fluorescent Light>>

In the case where the light-emitting substance used in the light-emitting layers (113, 113a, 113b, or 113c) is a fluorescent light-emitting material, an organic compound (a host material) used in combination with the light-emitting substance is preferably an organic compound that has a high energy level in a singlet excited state and has a low energy level in a triplet excited state or an organic compound having a fluorescence quantum yield. Therefore, the hole-transport material (described above) or the electron-transport material (described below) shown in this embodiment, for example, can be used as long as they are organic compounds that satisfy such a condition.

Examples of the organic compound in terms of a combination with a light-emitting substance (fluorescent light-emitting substance) include condensed polycyclic aromatic compounds such as an anthracene derivative, a tetracene derivative, a phenanthrene derivative, a pyrene derivative, a chrysene derivative, and a dibenzo[g,p]chrysene derivative, part of which are the same as the above-described compounds.

Specific examples of the organic compound (the host materials) preferably used in combination with a fluorescent light-emitting material include 9-phenyl-3-[4-(10-phenyl-9-anthryl)phenyl]-9H-carbazole (abbreviation: PCzPA), 3,6-diphenyl-9-[4-(10-phenyl-9-anthryl)phenyl]-9H-carbazole (abbreviation: DPCzPA), 3-[4-(1-naphthyl)-phenyl]-9-phenyl-9H-carbazole (abbreviation: PCPN), 9,10-diphenylanthracene (abbreviation: DPAnth), N,N-diphenyl-9-[4-(10-phenyl-9-anthryl)phenyl]-9H-carbazol-3-amine (abbreviation: CzA1PA), 4-(10-phenyl-9-anthryl)triphenylamine (abbreviation: DPhPA), YGAPA, PCAPA, N,9-diphenyl-N-{4-[4-(10-phenyl-9-anthryl)phenyl]phenyl}-9H-carbazol-3-amine (abbreviation: PCAPBA), N-(9,10-diphenyl-2-anthryl)-N,9-diphenyl-9H-carbazol-3-amine (abbreviation: 2PCAPA), 6,12-dimethoxy-5,11-diphenylchrysene, N,N,N′,N′,N″,N″,N′″,N′″-octaphenyldibenzo[g,p]chrysene-2,7,10,15-tetraamine (abbreviation: DBC1), 9-[4-(10-phenyl-9-anthracenyl)phenyl]-9H-carbazole (abbreviation: CzPA), 7-[4-(10-phenyl-9-anthryl)phenyl]-7H-dibenzo[c,g]carbazole (abbreviation: cgDBCzPA), 6-[3-(9,10-diphenyl-2-anthryl)phenyl]-benzo[b]naphtho[1,2-d]furan (abbreviation: 2mBnfPPA), 9-phenyl-10-{4-(9-phenyl-9H-fluoren-9-yl)-biphenyl-4′-yl}-anthracene (abbreviation: FLPPA), 9,10-bis(3,5-diphenylphenyl)anthracene (abbreviation: DPPA), 9,10-di(2-naphthyl)anthracene (abbreviation: DNA), 2-tert-butyl-9,10-di(2-naphthyl)anthracene (abbreviation: t-BuDNA), 9,9′-bianthryl (abbreviation: BANT), 9,9′-(stilbene-3,3′-diyl)diphenanthrene (abbreviation: DPNS), 9,9′-(stilbene-4,4′-diyl)diphenanthrene (abbreviation: DPNS2), 1,3,5-tri(1-pyrenyl)benzene (abbreviation: TPB3), and 5,12-diphenyltetracene, 5,12-bis(biphenyl-2-yl)tetracene.

### <<Host Material for Phosphorescent Light>>

In the case where the light-emitting substance used for the light-emitting layers (113, 113a, 113b, and 113c) is a phosphorescent material, an organic compound having triplet excitation energy (energy difference between a ground state and a triplet excited state) which is higher than that of the light-emitting substance is preferably selected as the organic compound (the host material) used in combination with the light-emitting substance. Note that in the case where a plurality of organic compounds (e.g., a first host material and a second host material (or an assist material)) are used in combination with a light-emitting substance in order to form an exciplex, the plurality of organic compounds are preferably mixed with a phosphorescent material.

Such a structure makes it possible to efficiently obtain light emission utilizing ExTET (Exciplex-Triplet Energy Transfer), which is energy transfer from an exciplex to a light-emitting substance. Note that a combination of the plurality of organic compounds that easily forms an exciplex is preferably employed, and it is particularly preferable to combine a compound that easily accepts holes (a hole-transport material) and a compound that easily accepts electrons (an electron-transport material).

In terms of a preferable combination with a light-emitting substance (phosphorescent substance), examples of the organic compound (the host material or the assist material) include an aromatic amine, a carbazole derivative, a dibenzothiophene derivative, a dibenzofuran derivative, a zinc- or aluminum-based metal complex, an oxadiazole derivative, a triazole derivative, a benzimidazole derivative, a quinoxaline derivative, a dibenzoquinoxaline derivative, a pyrimidine derivative, a triazine derivative, a pyridine derivative, a bipyridine derivative, and a phenanthroline derivative, part of which are the same as the above-described specific examples.

Specific examples of the aromatic amine among the above (a compound having an aromatic amine skeleton) and the carbazole derivative, which are organic compounds having a high hole-transport property, can be the same as those of the hole-transport material shown above. They are preferably used as a host material.

Among them, specific examples of the dibenzothiophene derivative and the dibenzofuran derivative, which are organic compounds having a high hole-transport property, include 4-{3-[3-(9-phenyl-9H-fluoren-9-yl)phenyl]phenyl}dibenzofuran (abbreviation: mmDBFFLBi-II), 4,4′,4″-(benzene-1,3,5-triyl)tri(dibenzofuran) (abbreviation: DBF3P-II), DBT3P-II, 2,8-diphenyl-4-[4-(9-phenyl-9H-fluoren-9-yl)phenyl]dibenzothiophene (abbreviation: DBTFLP-III), 4-[4-(9-phenyl-9H-fluoren-9-yl)phenyl]-6-phenyldibenzothiophene (abbreviation: DBTFLP-IV), and 4-[3-(triphenylen-2-yl)phenyl]dibenzothiophene (abbreviation: mDBTPTp-II), which are preferably used as a host material.

Among them, specific examples of metal complexes of the zinc- and aluminum-based metal complexes, which are organic compounds having a high electron-transport property (electron-transport material), include metal complexes including a quinoline skeleton or a benzoquinoline skeleton, tris(8-quinolinolato)aluminum(III) (abbreviation: Alq), tris(4-methyl-8-quinolinolato)aluminum(III) (abbreviation: Almq₃), bis(10-BeBq₂), hydroxybenzo[h]quinolinato)beryllium(II) (abbreviation: bis(2-methyl-8-quinolinolato)(4-phenylphenolato)aluminum(III) (abbreviation: BAlq), and bis(8-quinolinolato)zinc(II) (abbreviation: Znq), which are preferably used as a host material.

In addition to the above, a metal complex having an oxazole-based or thiazole-based ligand, such as bis[2-(2-benzoxazolyl)phenolato]zinc(II) (abbreviation: ZnPBO) or bis[2-(2-benzothiazolyl)phenolato]zinc(II) (abbreviation: ZnBTZ), or the like can also be used as a preferable host material.

Among them, specific examples of the oxadiazole derivative, the triazole derivative, the benzimidazole derivative, the quinoxaline derivative, the dibenzoquinoxaline derivative, and the phenanthroline derivative, which are organic compounds having a high electron-transport property (electron-transport material), include 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 3-(4-biphenylyl)-5-(4-tert-butylphenyl)-4-phenyl-1,2,4-triazole (abbreviation: TAZ), 2,2′,2″-(1,3,5-benzenetriyl)tris(1-phenyl-1H-benzimidazole) (abbreviation: TPBI), 2-[3-(dibenzothiophen-4-yl)phenyl]-1-phenyl-1H-benzimidazole (abbreviation: mDBTBIm-II), 4,4′-bis(5-methylbenzoxazol-2-yl)stilbene (abbreviation: BzOS), bathophenanthroline (abbreviation: Bphen), bathocuproine (abbreviation: BCP), 2,9-bis(naphthalen-2-yl)-4,7-diphenyl-1,10-phenanthroline (abbreviation: NBphen), 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 2-[4-(3,6-diphenyl-9H-carbazol-9-yl)phenyl]dibenzo[f,h]quinoxaline (abbreviation: 2CzPDBq-III), 7-[3-(dibenzothiophen-4-yl)phenyl]dibenzo[f,h]quinoxaline (abbreviation: 7mDBTPDBq-II), and 6-[3-(dibenzothiophen-4-yl)phenyl]dibenzo[f, h]quinoxaline (abbreviation: 6mDBTPDBq-II). These materials are preferably used as a host material.

Among them, specific examples of a heterocyclic compound having a diazine skeleton, a heterocyclic compound having a triazine skeleton, and a heterocyclic compound having a pyridine skeleton, which are organic compounds having a high electron-transport property (electron-transport material), include 4,6-bis[3-(phenanthren-9-yl)phenyl]pyrimidine (abbreviation: 4,6mPnP2Pm), 4,6-bis[3-(4-dibenzothienyl)phenyl]pyrimidine (abbreviation: 4,6mDBTP2Pm-II), 4,6-bis[3-(9H-carbazol-9-yl)phenyl]pyrimidine (abbreviation: 4,6mCzP2Pm), 2-{4-[3-(N-phenyl-9H-carbazol-3-yl)-9H-carbazol-9-yl]phenyl}-4,6-diphenyl-1,3,5-triazine (abbreviation: PCCzPTzn), 9-[3-(4,6-diphenyl-1,3,5-triazin-2-yl)phenyl]-9′-phenyl-2,3′-bi-9H-carbazole (abbreviation: mPCCzPTzn-02), 3,5-bis[3-(9H-carbazol-9-yl)phenyl]pyridine (abbreviation: 35DCzPPy), and 1,3,5-tri[3-(3-pyridyl)phenyl]benzene (abbreviation: TmPyPB). These materials are preferably used as a host material.

Furthermore, a high molecular compound such as poly(2,5-pyridinediyl) (abbreviation: PPy), poly[(9,9-dihexylfluorene-2,7-diyl)-co-(pyridine-3,5-diyl)] (abbreviation: PF-Py), or poly[(9,9-dioctylfluorene-2,7-diyl)-co-(2,2′-bipyridine-6,6′-diyl)] (abbreviation: PF-BPy), or the like is preferably used as a host material.

Furthermore, for example, 9-phenyl-9′-(4-phenyl-2-quinazolinyl)-3,3′-bi-9H-carbazole (abbreviation: PCCzQz) having bipolar properties, which is an organic compound having a high hole-transport property and a high electron-transport property, can be used as the host material.

### <Electron-Transport Layer>

The electron-transport layers (114, 114a, and 114b) are each a layer that transports the electrons, which are injected from the second electrode 102 and the charge-generation layers (106, 106a, and 106b) by the electron-injection layers (115, 115a, and 115b) described later, to the light-emitting layers (113, 113a, and 113b). Note that the electron-transport layers (114, 114a, and 114b) are each a layer containing an electron-transport material. It is preferable that the electron-transport materials used in the electron-transport layers (114, 114a, and 114b) be substances with an electron mobility higher than or equal to 1×10⁻⁶ cm²/Vs in the case where the square root of the electric field strength [V/cm] is 600. Note that other substances can be used as long as they have a property of transporting more electrons than holes. Electron-transport layers (114, 114a, and 114b) each function even with a single-layer structure, but can improve the device characteristics when having a stacked-layer structure of two or more layers as needed.

### <<Electron-Transport Material>>

As the hole-transport material that can be used for the electron-transport layers (114, 114a, and 114b), it is possible to use, a material having a high electron-transport property (electron-transport material) such as, in addition to the organic compounds having a structure in which an aromatic ring is fused to a furan ring of a furodiazine skeleton, a metal complex having a quinoline skeleton, a metal complex having a benzoquinoline skeleton, a metal complex having an oxazole skeleton, a metal complex having a thiazole skeleton, an oxadiazole derivative, a triazole derivative, an imidazole derivative, an oxazole derivative, a thiazole derivative, a phenanthroline derivative, a quinoline derivative having a quinoline ligand, a benzoquinoline derivative, a quinoxaline derivative, a dibenzoquinoxaline derivative, a pyridine derivative, a bipyridine derivative, a pyrimidine derivative, or a π-electron deficient heteroaromatic compound such as a nitrogen-containing heteroaromatic compound.

Specific examples of the electron-transport material include metal complexes having a quinoline skeleton or a benzoquinoline skeleton, such as 2-[3′-(dibenzothiophen-4-yl)biphenyl-3-yl]dibenzo[f,h]quinoxaline (abbreviation: 2mDBTBPDBq-II), 5-[3-(4,6-diphenyl-1,3,5-triazin-2yl)phenyl1]-7,7-dimethyl-5H,7H-indeno[2,1-b]carbazole (abbreviation: mINc(II)PTzn), 2-{3-[3-(dibenzothiophen-4-yl)phenyl]phenyl}-4,6-diphenyl-1,3,5-tri azine (abbreviation: mDBtBPTzn), 4-[3-(dibenzothiophen-4-yl)phenyl]-8-(naphthalen-2-yl)-[1]benzofuro[3,2-d]pyrimidine (abbreviation: 8βN-4mDBtPBfpm), 3,8-bis[3-(dibenzothiophen-4-yl)phenyl]benzofuro[2,3-b]pyrazine (abbreviation: 3,8mDBtP2Bfpr), 4,8-bis[3-(dibenzothiophen-4-yl)phenyl]-[1]benzofuro[3,2-d]pyrimidine (abbreviation: 4,8mDBtP2Bfpm), 9-[(3′-dibenzothiophen-4-yl)biphenyl-3-yl]naphtho[1′,2′:4,5]furo[2,3-b]pyrazine (abbreviation: 9mDBtBPNfpr), 8-[3′-(dibenzothiophen-4-yl)(1,1′-biphenyl-3-yl)]naphtho[1′,2′:4,5]furo[3,2-d]pyrimidine (abbreviation: 8mDBtBPNfpm), 8-[(2,2′-binaphthalen)-6-yl]-4-[3-(dibenzothiophen-4-yl)phenyl]-[1]benzofuro[3,2-d]pyrimidine (abbreviation: 8 (βN2)-4mDBtPBfpm), 8-(1,1′-biphenyl-4-yl)-4-[3-(dibenzothiophen-4-yl)phenyl]-[1]benzofuro[3,2-d]pyrimidine (abbreviation: 8BP-4mDBtPBfpm), tris(8-quinolinolato)aluminum(III) (abbreviation: Alq₃), Almq₃, BeBq₂, bis(2-methyl-8-quinolinolato)(4-phenylphenolato)aluminum(III) (abbreviation: BAlq), and bis(8-quinolinolato)zinc(II) (abbreviation: Znq), and metal complexes having an oxazole skeleton or a thiazole skeleton, such as bis[2-(2-benzoxazolyl)phenolato]zinc(II) (abbreviation: ZnPBO) and bis[2-(2-benzothiazolyl)phenolato]zinc(II) (abbreviation: ZnBTZ).

Other than the metal complexes, as the electron-transport material, it is possible to use oxadiazole derivatives such as PBD, OXD-7, and CO11, triazole derivatives such as TAZ and p-EtTAZ, imidazole derivatives (including benzimidazole derivatives) such as TPBI and mDBTBIm-II, an oxazole derivative such as BzOs, phenanthroline derivatives such as Bphen, BCP, and NBphen, quinoxaline derivatives and dibenzoquinoxaline derivatives, such as 2mDBTPDBq-II, 2mDBTBPDBq-II, 2mCzBPDBq, 2CzPDBq-III, 7mDBTPDBq-II, and 6mDBTPDBq-II, pyridine derivatives such as 35DCzPPy and TmPyPB, pyrimidine derivatives such as 4,6mPnP2Pm, 4,6mDBTP2Pm-II, and 4,6mCzP2Pm, and triazine derivatives such as PCCzPTzn and mPCCzPTzn-02.

Furthermore, a high molecular compound such as poly(2,5-pyridinediyl) (abbreviation: PPy), poly[(9,9-dihexylfluorene-2,7-diyl)-co-(pyridine-3,5-diyl)] (abbreviation: PF-Py), or poly[(9,9-dioctylfluorene-2,7-diyl)-co-(2,2′-bipyridine-6,6′-diyl)] (abbreviation: PF-BPy) can be used as the electron-transport material.

Each of the electron-transport layers (114, 114a, or 114b) is not limited to a single layer, and may be a stack of two or more layers each made of any of the above substances.

### <Electron-Injection Layer>

The electron-injection layers (115, 115a, and 115b) are each a layer containing a substance having a high electron-injection property. The electron-injection layers (115, 115a, and 115b) are each a layer for increasing the efficiency of electron injection from the second electrode 102 and is preferably formed using a material whose LUMO level value has a small difference (0.5 eV or less) from the work function value of the material used for the second electrode 102. Thus, the electron-injection layers (115, 115a, and 115b) can be formed using an alkali metal, an alkaline earth metal, or a compound thereof, such as lithium, cesium, lithium fluoride (LiF), cesium fluoride (CsF), calcium fluoride (CaF₂), 8-(quinolinolato)lithium (abbreviation: Liq), 2-(2-pyridyl)phenolatolithium (abbreviation: LiPP), 2-(2-pyridyl)-3-pyridinolatolithium (abbreviation: LiPPy), 4-phenyl-2-(2-pyridyl)phenolatolithium (abbreviation: LiPPP), lithium oxide (LiOₓ), or cesium carbonate. A rare earth metal compound like erbium fluoride (ErF₃) can also be used. Electride may also be used for the electron-injection layers (115, 115a, and 115b). Examples of the electride include a substance in which electrons are added at high concentration to a mixed oxide of calcium and aluminum. Note that any of the substances used in the electron-transport layers (114, 114a, and 114b), which are given above, can also be used.

A composite material in which an organic compound and an electron donor (donor) are mixed may also be used in the electron-injection layers (115, 115a, and 115b). Such a composite material is excellent in an electron-injection property and an electron-transport property because electrons are generated in the organic compound by the electron donor. In this case, the organic compound is preferably a material excellent in transporting the generated electrons; specifically, for example, the above-mentioned electron-transport materials (metal complexes, heteroaromatic compounds, and the like) used in the electron-transport layers (114, 114a, and 114b) can be used. As the electron donor, a substance showing an electron-donating property with respect to the organic compound may be used. Specifically, an alkali metal, an alkaline earth metal, and a rare earth metal are preferable, and lithium, cesium, magnesium, calcium, erbium, ytterbium, and the like are given. In addition, an alkali metal oxide and an alkaline earth metal oxide are preferable, and lithium oxide, calcium oxide, barium oxide, and the like are given. A Lewis base such as magnesium oxide can be used. Furthermore, an organic compound such as tetrathiafulvalene (abbreviation: TTF) can be used.

Moreover, a composite material in which an organic compound and a metal are mixed may also be used in the electron-injection layers (115, 115a, and 115b). The organic compound used here preferably has a LUMO level higher than or equal to −3.6 eV and lower than or equal to −2.3 eV. Moreover, a material having an unshared electron pair is preferable.

Therefore, the above organic compound is preferably a material having an unshared electron pair, such as a heterocyclic compound having a pyridine skeleton, a diazine skeleton (e.g., a pyrimidine skeleton or a pyrazine skeleton), or a triazine skeleton.

Examples of the heterocyclic compound having a pyridine skeleton include 3,5-bis[3-(9H-carbazol-9-yl)phenyl]pyridine (abbreviation: 35DCzPPy), 1,3,5-tri[3-(3-pyridyl)phenyl]benzene (abbreviation: TmPyPB), bathocuproine (abbreviation: BCP), 2,9-bis(naphthalen-2-yl)-4,7-diphenyl-1,10-phenanthroline (abbreviation: NBPhen), and bathophenanthroline (abbreviation: Bphen).

Examples of the heterocyclic compound having a diazine skeleton include 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 2-[4-(3,6-diphenyl-9H-carbazol-9-yl)phenyl]dibenzo[f, h]quinoxaline (abbreviation: 2CzPDBq-III), 7-[3-(dibenzothiophen-4-yl)phenyl]dibenzo[f,h]quinoxaline (abbreviation: 7mDBTPDBq-II), 6-[3-(dibenzothiophen-4-yl)phenyl]dibenzo[f,h]quinoxaline (abbreviation: 6mDBTPDBq-II), 4,6-bis[3-(phenanthren-9-yl)phenyl]pyrimidine (abbreviation: 4,6mPnP2Pm), 4,6-bis[3-(4-dibenzothienyl)phenyl]pyrimidine (abbreviation: 4,6mDBTP2Pm-II), 4,6-bis[3-(9H-carbazol-9-yl)phenyl]pyrimidine (abbreviation: 4,6mCzP2Pm), and 4-{3-[3′-(9H-carbazol-9-yl)]biphenyl-3-yl}benzofuro[3,2-d]pyrimidine (abbreviation: 4mCzBPBfpm).

Examples of the heterocyclic compound having a triazine skeleton include 2-{4-[3-(N-phenyl-9H-carbazol-3-yl)-9H-carbazol-9-yl]phenyl}-4,6-diphenyl-1,3,5-tri azine (abbreviation: PCCzPTzn), 2,4,6-tris[3′-(pyridin-3-yl)biphenyl-3-yl]-1,3,5-triazine (abbreviation: TmPPPyTz), and 2,4,6-tris(2-pyridyl)-1,3,5-triazine (abbreviation: 2Py3Tz).

As a metal, a transition metal that belongs to Group 5, Group 7, Group 9, or Group 11 or a material that belongs to Group 13 in the periodic table is preferably used, and examples include Ag, Cu, Al, and In. In this case, the organic compound forms a singly occupied molecular orbital (SOMO) with the transition metal.

Note that in the case where light obtained from the light-emitting layer 113b is amplified, for example, formation is preferably performed such that the optical path length between the second electrode 102 and the light-emitting layer 113b is less than one fourth of the wavelength of light emitted from the light-emitting layer 113b. In that case, the optical path length can be adjusted by changing the thickness of the electron-transport layer 114b or the electron-injection layer 115b.

When the charge-generation layer 106 is provided between two EL layers (103a and 103b) as in the light-emitting device illustrated in FIG. 1D, a structure in which a plurality of EL layers are stacked between the pair of electrodes (also referred to as a tandem structure) can be employed.

### <Charge-Generation Layer>

The charge-generation layer 106 has a function of injecting electrons into the EL layer 103a and injecting holes into the EL layer 103b when voltage is applied between the first electrode (anode) 101 and the second electrode (cathode) 102. Note that the charge-generation layer 106 may have either a structure in which an electron acceptor (acceptor) is added to a hole-transport material or a structure in which an electron donor (donor) is added to an electron-transport material. Alternatively, both of these structures may be stacked. Note that forming the charge-generation layer 106 with use of any of the above materials can inhibit an increase in drive voltage in the case where the EL layers are stacked.

In the case where the charge-generation layer 106 has a structure in which an electron acceptor is added to a hole-transport material that is an organic compound, any of the materials described in this embodiment can be used as the hole-transport material. As the electron acceptor, 7,7,8,8-tetracyano-2,3,5,6-tetrafluoroquinodimethane (abbreviation: F4-TCNQ), chloranil, and the like can be given. Other examples include oxides of metals belonging to Group 4 to Group 8 of the periodic table. Specific examples are vanadium oxide, niobium oxide, tantalum oxide, chromium oxide, molybdenum oxide, tungsten oxide, manganese oxide, and rhenium oxide.

In the case where the charge-generation layer 106 has a structure in which an electron donor is added to an electron-transport material, any of the materials described in this embodiment can be used as the electron-transport material. As the electron donor, it is possible to use an alkali metal, an alkaline earth metal, a rare earth metal, metals belonging to Group 2 and Group 13 of the periodic table, or an oxide or carbonate thereof. Specifically, lithium (Li), cesium (Cs), magnesium (Mg), calcium (Ca), ytterbium (Yb), indium (In), lithium oxide, cesium carbonate, or the like is preferably used. An organic compound such as tetrathianaphthacene may be used as the electron donor.

Although FIG. 1D illustrates the structure in which two EL layers 103 are stacked, three or more EL layers may be stacked with a charge-generation layer provided between different EL layers.

### <Substrate>

The light-emitting device described in this embodiment can be formed over any of a variety of substrates. Note that the type of the substrate is not limited to a certain type. Examples of the substrate include semiconductor substrates (e.g., a single crystal substrate and a silicon substrate), an SOI substrate, a glass substrate, a quartz substrate, a plastic substrate, a metal substrate, a stainless steel substrate, a substrate including stainless steel foil, a tungsten substrate, a substrate including tungsten foil, a flexible substrate, a laminate film, paper including a fibrous material, and a base material film.

Note that examples of the glass substrate include barium borosilicate glass, aluminoborosilicate glass, and soda lime glass. Examples of the flexible substrate, the attachment film, and the base material film include plastics typified by polyethylene terephthalate (PET), polyethylene naphthalate (PEN), and polyether sulfone (PES); a synthetic resin such as acrylic; polypropylene; polyester; polyvinyl fluoride; polyvinyl chloride; polyamide; polyimide; aramid; epoxy; an inorganic vapor deposition film; and paper.

For fabrication of the light-emitting device in this embodiment, a vacuum process such as an evaporation method or a solution process such as a spin coating method or an ink-jet method can be used. In the case of using an evaporation method, a physical vapor deposition method (PVD method) such as a sputtering method, an ion plating method, an ion beam evaporation method, a molecular beam evaporation method, or a vacuum evaporation method, a chemical vapor deposition method (CVD method), or the like can be used. Specifically, the layers having a variety of functions (the hole-injection layers (111, 111a, and 111b), the hole-transport layers (112, 112a, and 112b), the light-emitting layers (113, 113a, 113b, and 113c), the electron-transport layers (114, 114a, and 114b), and the electron-injection layers (115, 115a, and 115b)) included in the EL layers and the charge-generation layers (106, 106a, and 106b) of the light-emitting device can be formed by an evaporation method (e.g., a vacuum evaporation method), a coating method (e.g., a dip coating method, a die coating method, a bar coating method, a spin coating method, or a spray coating method), a printing method (e.g., an ink-jet method, a screen printing (stencil) method, an offset printing (planography) method, a flexography (relief printing) method, a gravure printing method, or a micro-contact printing method), or the like.

For example, in the case of using the above deposition method including the coating method or printing method, a high molecular compound (e.g., an oligomer, a dendrimer, and a polymer), a middle molecular compound (a compound between a low molecular compound and a high molecular compound, with a molecular weight of 400 to 4000), or an inorganic compound (e.g., a quantum dot material) can be used. Note that as the quantum dot material, a colloidal quantum dot material, an alloyed quantum dot material, a core-shell quantum dot material, a core quantum dot material, or the like can be used.

Note that materials that can be used for the layers (the hole-injection layers (111, 111a, and 111b), the hole-transport layers (112, 112a, and 112b), the light-emitting layers (113, 113a, 113b, and 113c), the electron-transport layers (114, 114a, and 114b), and the electron-injection layers (115, 115a, and 115b)) included in the EL layers (103, 103a, and 103b) and the charge-generation layers (106, 106a, and 106b) of the light-emitting device described in this embodiment are not limited to the materials shown in this embodiment, and other materials can also be used in combination as long as the functions of the layers are fulfilled.

The structure described in this embodiment can be used in an appropriate combination with any of the structures described in the other embodiments.

### Embodiment 3

In this embodiment, a specific structure example of a light-emitting apparatus (also referred to as display panel) of one embodiment of the present invention and a manufacturing method thereof will be described.

### Structure Example 1 of Light-Emitting Apparatus 700

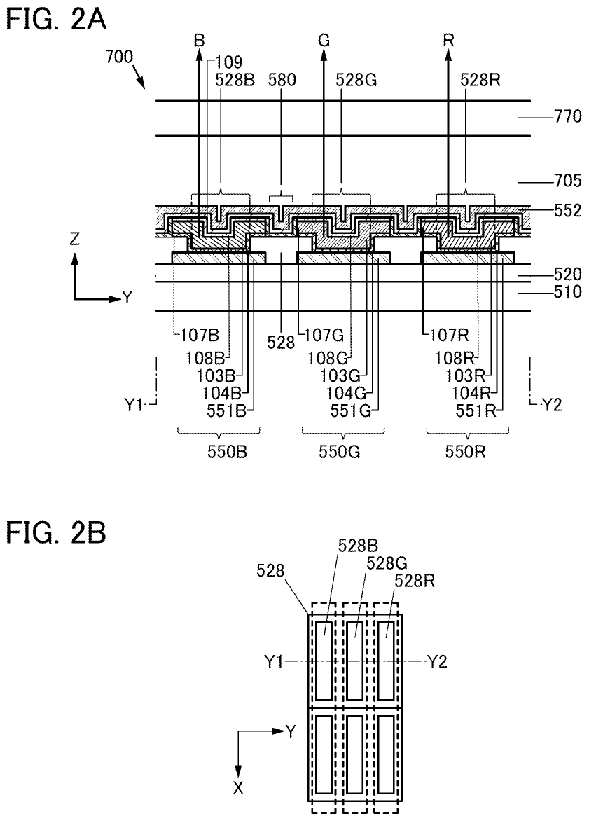

A light-emitting apparatus 700 illustrated in FIG. 2A includes a light-emitting device 550B, a light-emitting device 550G, a light-emitting device 550R, and a partition 528. The light-emitting device 550B, the light-emitting device 550G, the light-emitting device 550R, and the partition 528 are formed over a functional layer 520 provided over a first substrate 510. The functional layer 520 includes, for example, a gate line driver circuit GD, a source line driver circuit SD, and the like that are composed of a plurality of transistors, and wirings that electrically connect these circuits. The driver circuit GD and the driver circuit SD will be described in Embodiment 4. Note that these driver circuits are electrically connected to the light-emitting device 550B, the light-emitting device 550G, and the light-emitting device 550R, for example, to drive them. The light-emitting apparatus 700 includes an insulating layer 705 over the functional layer 520 and the light-emitting devices, and the insulating layer 705 has a function of attaching a second substrate 770 and the functional layer 520.

The light-emitting device 550B, the light-emitting device 550G, and the light-emitting device 550R each have the device structure described in Embodiment 2. Specifically, the case is described in which the EL layer 103 in the structure illustrated in FIG. 1A differs between the light-emitting devices.

The light-emitting device 550B includes an electrode 551B, an electrode 552, an EL layer 103B, and an insulating layer 107B. Note that a specific structure of each layer is as described in Embodiment 2. The EL layer 103B has a stacked-layer structure of layers having different functions including a light-emitting layer. Although FIG. 2A illustrates only a hole-injection/transport layer 104B, an electron-transport layer 108B, and an electron-injection layer 109 in the layers included in the EL layer 103B, which includes the light-emitting layer, the present invention is not limited thereto. Note that the hole-injection/transport layer 104B represents the layer having the functions of the hole-injection layer and the hole-transport layer described in Embodiment 1 and may have a stacked-layer structure. Note that in this specification, a hole-injection/transport layer in any light-emitting device can be interpreted in the above manner. The electron-transport layer may have a stacked-layer structure, and may include a hole-blocking layer, in contact with the light-emitting layer, which blocks holes moving from the anode side to the cathode side through the light-emitting layer. The electron-injection layer 109 may have a stacked-layer structure in which some or all of layers are formed using different materials.

As illustrated in FIG. 2A, the insulating layer 107B is formed while a resist formed over some layers of the EL layer 103B (in this embodiment, the layers up to the electron-transport layer 108B over the light-emitting layer) remains over the electrode 551B. Thus, the insulating layer 107B is formed in contact with a side surface (or an end portion) of the EL layer 103B. This can inhibit entry of oxygen, moisture, or constituent elements thereof into the inside through the side surface of the EL layer 103B. For the insulating layer 107B, aluminum oxide, magnesium oxide, hafnium oxide, gallium oxide, indium gallium zinc oxide, silicon nitride, silicon nitride oxide, or the like can be used, for example. The insulating layer 107B can be formed by a sputtering method, a CVD method, an MBE method, a PLD method, an ALD method, or the like and is formed preferably by an ALD method, which achieves favorable coverage.

The electron-injection layer 109 is formed to cover part of the EL layer 103B (the electron-transport layer 108B) and the insulating layer 107B. The electron-injection layer 109 preferably has a stacked-layer structure of two or more layers having different electric resistances. For example, the electron-injection layer 109 may have one of the following structures: a structure in which a first layer in contact with the electron-transport layer 108B is formed using only an electron-transport material, and a second layer formed using an electron-transport material containing a metal material is stacked over the first layer; or the aforementioned structure including a third layer formed using an electron-transport material containing a metal material, between the first layer and the electron-transport layer 108B.

The electrode 552 is formed over the electron-injection layer 109. Note that the electrode 551B and the electrode 552 have an overlap region. The EL layer 103B is positioned between the electrode 551B and the electrode 552. Thus, the electron-injection layer 109 is in contact with the side surfaces (or end portions) of the EL layer 103B with the insulating layer 107 therebetween, or the electrode 552 is in contact with the side surfaces (or end portions) of the EL layer 103B with the electron-injection layer 109 and the insulating layer 107B therebetween. Hence, the EL layer 103B and the electrode 552, specifically the hole-injection/transport layer 104B in the EL layer 103B and the electrode 552 can be prevented from being electrically short-circuited.

The EL layer 103B illustrated in FIG. 2A has a structure similar to those of the EL layers 103, 103a, 103b, and 103c described in Embodiment 1. The EL layer 103B is capable of emitting blue light, for example.

The light-emitting device 550G includes an electrode 551G, the electrode 552, an EL layer 103G, and the insulating layer 107. Note that a specific structure of each layer is as described in Embodiment 2. The EL layer 103G has a stacked-layer structure of layers having different functions including a light-emitting layer. Although FIG. 2A illustrates only a hole-injection/transport layer 104G, an electron-transport layer 108G, and the electron-injection layer 109 in the layers included in the EL layer 103G, which includes the light-emitting layer, the present invention is not limited thereto. Note that the hole-injection/transport layer 104G represents the layer having the functions of the hole-injection layer and the hole-transport layer described in Embodiment 2 and may have a stacked-layer structure.

The electron-transport layer may have a stacked-layer structure, and may include a hole-blocking layer, in contact with the light-emitting layer, which blocks holes moving from the anode side to the cathode side through the light-emitting layer. The electron-injection layer 109 may have a stacked-layer structure in which some or all of layers are formed using different materials.

As illustrated in FIG. 2A, an insulating layer 107G is formed while a resist formed over some layers of the EL layer 103G (in this embodiment, the layers up to the electron-transport layer 108G over the light-emitting layer) remains over the electrode 551G. Thus, the insulating layer 107G is formed in contact with part (the above) of a side surface (or an end portion) of the EL layer 103G. Accordingly, entry of oxygen, moisture, or constituent elements thereof through the side surface of the EL layer 103G into the inside of the EL layer 103G can be inhibited. For the insulating layer 107G, aluminum oxide, magnesium oxide, hafnium oxide, gallium oxide, indium gallium zinc oxide, silicon nitride, silicon nitride oxide, or the like can be used, for example. The insulating layer 107 can be formed by a sputtering method, a CVD method, an MBE method, a PLD method, an ALD method, or the like and is formed preferably by an ALD method, which achieves favorable coverage.

The electron-injection layer 109 is formed to cover part of the EL layer 103G (the electron-transport layer 108G) and the insulating layer 107G. The electron-injection layer 109 preferably has a stacked-layer structure of two or more layers having different electric resistances. For example, the electron-injection layer 109 may have one of the following structures: a structure in which a first layer in contact with the electron-transport layer 108G is formed using only an electron-transport material, and a second layer formed using an electron-transport material containing a metal material is stacked over the first layer; or the aforementioned structure including a third layer formed using an electron-transport material containing a metal material, between the first layer and the electron-transport layer 108G.

The electrode 552 is formed over the electron-injection layer 109. Note that the electrode 551G and the electrode 552 have an overlap region. The EL layer 103G is positioned between the electrode 551G and the electrode 552. Thus, the electron-injection layer 109 is in contact with the side surfaces (or end portions) of the EL layer 103G with the insulating layer 107 therebetween, or the electrode 552 is in contact with the side surfaces (or end portions) of the EL layer 103G with the electron-injection layer 109 and the insulating layer 107G therebetween. Hence, the EL layer 103G and the electrode 552, specifically the hole-injection/transport layer 104G in the EL layer 103G and the electrode 552 can be prevented from being electrically short-circuited.

The EL layer 103G illustrated in FIG. 2A has the same structure as the EL layers 103, 103a, 103b, and 103c described in Embodiment 1. The EL layer 103G is capable of emitting green light, for example.

The light-emitting device 550R includes an electrode 551R, the electrode 552, an EL layer 103R, and an insulating layer 107R. Note that a specific structure of each layer is as described in Embodiment 2. The EL layer 103R has a stacked-layer structure of layers having different functions including a light-emitting layer. Although FIG. 2A illustrates only a hole-injection/transport layer 104R, an electron-transport layer 108R, and an electron-injection layer 109 in the layers included in the EL layer 103R, which includes the light-emitting layer, the present invention is not limited thereto. Note that the hole-injection/transport layer 104R represents the layer having the functions of the hole-injection layer and the hole-transport layer described in Embodiment 2 and may have a stacked-layer structure. Note that in this specification, a hole-injection/transport layer in any light-emitting device can be interpreted in the above manner. The electron-transport layer may have a stacked-layer structure, and may include a hole-blocking layer, in contact with the light-emitting layer, which blocks holes moving from the anode side to the cathode side through the light-emitting layer. The electron-injection layer 109 may have a stacked-layer structure in which some or all of layers are formed using different materials.

As illustrated in FIG. 2A, the insulating layer 107R is formed while a resist formed over some layers of the EL layer 103R (in this embodiment, the layers up to the electron-transport layer 108R over the light-emitting layer) remains over the electrode 551R. Thus, the insulating layer 107R is formed in contact with part (the above) of a side surface (or an end portion) of the EL layer 103R. Accordingly, entry of oxygen, moisture, or constituent elements thereof through the side surface of the EL layer 103R into the inside of the EL layer 103R can be inhibited. For the insulating layer 107R, aluminum oxide, magnesium oxide, hafnium oxide, gallium oxide, indium gallium zinc oxide, silicon nitride, silicon nitride oxide, or the like can be used, for example. The insulating layer 107R can be formed by a sputtering method, a CVD method, an MBE method, a PLD method, an ALD method, or the like and is formed preferably by an ALD method, which achieves favorable coverage.

The electron-injection layer 109 is formed to cover part of the EL layer 103R (the electron-transport layer 108R) and the insulating layer 107R. The electron-injection layer 109 preferably has a stacked-layer structure of two or more layers having different electric resistances. For example, the electron-injection layer 109 may have one of the following structures: a structure in which a first layer in contact with the electron-transport layer 108R is formed using only an electron-transport material, and a second layer formed using an electron-transport material containing a metal material is stacked over the first layer; or the aforementioned structure including a third layer formed using an electron-transport material containing a metal material, between the first layer and the electron-transport layer 108R.

The electrode 552 is formed over the electron-injection layer 109. Note that the electrode 551R and the electrode 552 have an overlap region. The EL layer 103R is positioned between the electrode 551R and the electrode 552. Thus, the electron-injection layer 109 is in contact with the side surfaces (or end portions) of some layers of the EL layer 103B with the insulating layer 107 therebetween, or the electrode 552 is in contact with the side surfaces (or end portions) of some layers of the EL layer 103B with the electron-injection layer 109 and the insulating layer 107B therebetween. Hence, the EL layer 103R and the electrode 552, specifically the hole-injection/transport layer 104R in the EL layer 103R and the electrode 552 can be prevented from being electrically short-circuited.

The EL layer 103R illustrated in FIG. 2A has a structure similar to that of the EL layers 103, 103a, 103b, and 103c described in Embodiment 1. The EL layer 103R is capable of emitting red light, for example.

A space 580 is provided between the EL layer 103B, the EL layer 103G, and the EL layer 103R. In each of the EL layers, particularly the hole-injection layer, which is included in the hole-transport region between the anode and the light-emitting layer, often has high conductivity;

therefore, a hole-injection layer formed as a layer shared by adjacent light-emitting devices might cause crosstalk. Thus, providing the space 580 between the EL layers as shown in this structure example can inhibit occurrence of crosstalk between adjacent light-emitting devices.

When electrical continuity is established between the EL layer 103B, the EL layer 103G, and the EL layer 103R in a light-emitting apparatus (display panel) with a high resolution exceeding 1000 ppi, a crosstalk phenomenon occurs, resulting in a narrower color gamut that the light-emitting apparatus is capable of reproducing. Providing the space 580 in a high-resolution display panel with more than 1000 ppi, preferably more than 2000 ppi, or further preferably in an ultrahigh-resolution display panel with more than 5000 ppi allows the display panel to express vivid colors.

As illustrated in FIG. 2B, the partition 528 has an opening 528B, an opening 528G, and an opening 528R. As illustrated in FIG. 2A, the opening 528B overlaps with the electrode 551B, the opening 528G overlaps with the electrode 551G, and the opening 528R overlaps with the electrode 551R. Note that a cross-sectional view taken along the dashed-dotted line Y1-Y2 in FIG. 2B corresponds to a schematic cross-sectional view of the light-emitting apparatus illustrated in FIG. 2A.

The EL layers (the EL layer 103B, the EL layer 103G, and the EL layer 103R) are processed to be separated by patterning using a photolithography method; hence, a high-resolution light-emitting apparatus (display panel) can be fabricated. End portions (side surfaces) of the EL layer processed by patterning using a photolithography method have substantially one surface (or are positioned on substantially the same plane). In this case, the space 580 between the EL layers is preferably 5 μm or less, further preferably 1 μm or less.

In the EL layer, particularly the hole-injection layer, which is included in the hole-transport region between the anode and the light-emitting layer, often has high conductivity; thus, a hole-injection layer formed as a layer shared by adjacent light-emitting devices might cause crosstalk. Therefore, processing the EL layers to be separated by patterning using a photolithography method as shown in this structure example can inhibit occurrence of crosstalk between adjacent light-emitting devices.

### Manufacturing Method Example 1 of Light-Emitting Apparatus

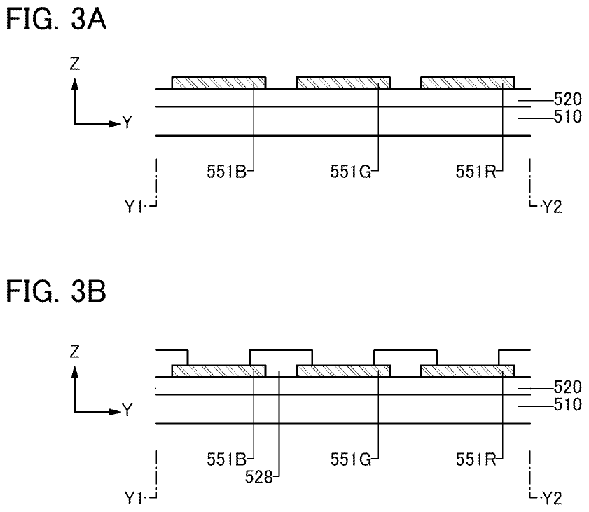

The electrode 551B, the electrode 551G, and the electrode 551R are formed as illustrated in FIG. 3A. For example, a conductive film is formed over the functional layer 520 over the first substrate 510 and processed into predetermined shapes by a photolithography method.

The conductive film can be formed by any of a sputtering method, a chemical vapor deposition (CVD) method, a vacuum evaporation method, a pulsed laser deposition (PLD) method, an atomic layer deposition (ALD) method, and the like. Examples of the CVD method include a plasma-enhanced chemical vapor deposition (PECVD) method and a thermal CVD method. An example of a thermal CVD method is a metal organic CVD (MOCVD) method.

The conductive film may be processed by a nanoimprinting method, a sandblasting method, a lift-off method, or the like as well as a photolithography method described above. Alternatively, island-shaped thin films may be directly formed by a deposition method using a shielding mask such as a metal mask.

In this specification and the like, a device formed using a metal mask or an FMM (a fine metal mask, a high-resolution metal mask) may be referred to as a device having an MM (a metal mask) structure. In this specification and the like, a device formed without using a metal mask or an FMM is sometimes referred to as a device having an MML (metal maskless) structure.

There are the following two typical examples of a photolithography method. In one of the methods, a resist mask is formed over a thin film that is to be processed, the thin film is processed by etching or the like, and then the resist mask is removed. In the other method, a photosensitive thin film is deposited and then processed into a desired shape by light exposure and development.

As light for exposure in a photolithography method, it is possible to use light with the i-line (wavelength: 365 nm), light with the g-line (wavelength: 436 nm), light with the h-line (wavelength: 405 nm), or combined light of any of them. Alternatively, ultraviolet light, KrF laser light, ArF laser light, or the like can be used. Exposure may be performed by liquid immersion exposure technique. As the light for exposure, extreme ultraviolet (EUV) light or X-rays may also be used. Instead of the light for exposure, an electron beam can be used. It is preferable to use extreme ultraviolet light, X-rays, or an electron beam because extremely minute processing can be performed. Note that a photomask is not needed when exposure is performed by scanning with a beam such as an electron beam.

For etching of a thin film using a resist mask, a dry etching method, a wet etching method, a sandblast method, or the like can be used.

Next, as illustrated in FIG. 3B, the partition 528 is formed between the electrode 551B, the electrode 551G, and the electrode 551R. For example, the partition 528 can be formed in such a manner that an insulating film covering the electrode 551B, the electrode 551G, and the electrode 551R is formed, and openings are formed by a photolithography method to partly expose the electrode 551B, the electrode 551G, and the electrode 551R. Examples of a material that can be used for the partition 528 include an inorganic material, an organic material, and a composite material of an inorganic material and an organic material. Specifically, it is possible to use an inorganic oxide film, an inorganic nitride film, an inorganic oxynitride film, or the like, or a layered material in which two or more films selected from the above are stacked. More specifically, it is possible to use a silicon oxide film, a film containing acrylic, a film containing polyimide, or the like, or a layered material in which two or more films selected from the above are stacked.

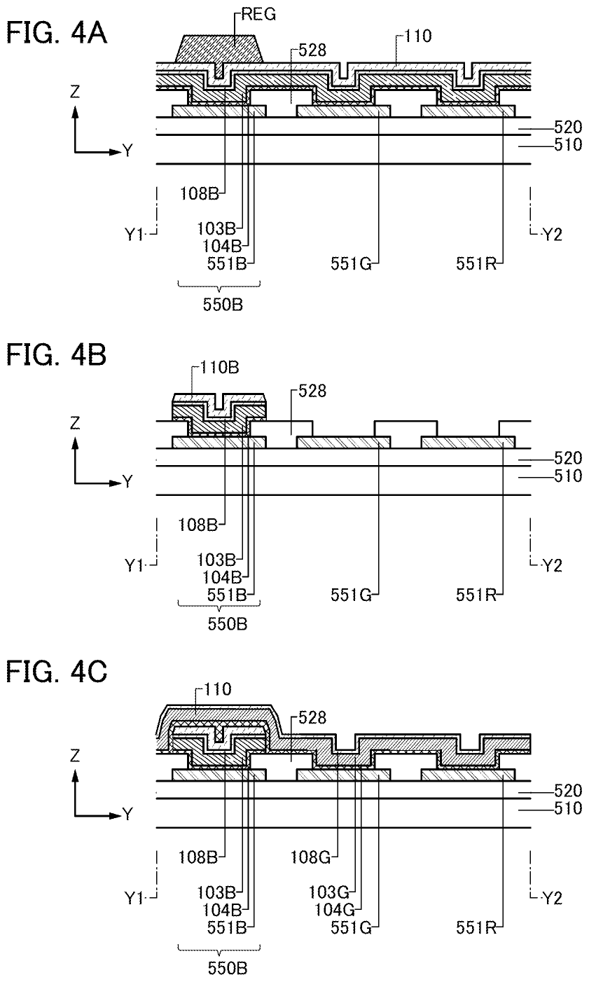

Then, as illustrated in FIG. 4A, the EL layer 103B is formed over the electrode 551B, the electrode 551G, the electrode 551R, and the partition 528. Note that in the EL layer 103B in FIG. 4A, the hole-injection/transport layer 104B, the light-emitting layer, and the electron-transport layer 108B are formed. For example, the EL layer 103B is formed by a vacuum evaporation method over the electrode 551B, the electrode 551G, the electrode 551R, and the partition 528 so as to cover them. Furthermore, a sacrificial layer 110 is formed over the EL layer 103B.

As the sacrificial layer 110, it is possible to use a film highly resistant to etching treatment performed on the EL layer 103B, i.e., a film having high etching selectivity. The sacrificial layer 110 preferably has a stacked-layer structure of a first sacrificial layer and a second sacrificial layer which have different etching selectivities. Moreover, the sacrificial layer 110, it is possible to use a film that can be removed by a wet etching method less likely to cause damage to the EL layer 103B.

The sacrificial layer 110 can be formed using an inorganic film such as a metal film, an alloy film, a metal oxide film, a semiconductor film, or an inorganic insulating film, for example. The sacrificial layer 110 can be formed by any of a variety of film formation methods such as a sputtering method, an evaporation method, a CVD method, and an ALD method.

For the sacrificial layer 110, a metal material such as gold, silver, platinum, magnesium, nickel, tungsten, chromium, molybdenum, iron, cobalt, copper, palladium, titanium, aluminum, yttrium, zirconium, or tantalum or an alloy material containing the metal material can be used. It is particularly preferable to use a low-melting-point material such as aluminum or silver.

Alternatively, the sacrificial layer 110 can be formed using a metal oxide such as an indium gallium zinc oxide (In—Ga—Zn oxide, also referred to as IGZO) or the like. It is also possible to use indium oxide, indium zinc oxide (In—Zn oxide), indium tin oxide (In—Sn oxide), indium titanium oxide (In—Ti oxide), indium tin zinc oxide (In—Sn—Zn oxide), indium titanium zinc oxide (In—Ti—Zn oxide), or indium gallium tin zinc oxide (In—Ga—Sn—Zn oxide). Alternatively, indium tin oxide containing silicon or the like can also be used.

Note that an element M (M is one or more of aluminum, silicon, boron, yttrium, copper, vanadium, beryllium, titanium, iron, nickel, germanium, zirconium, molybdenum, lanthanum, cerium, neodymium, hafnium, tantalum, tungsten, and magnesium) may be used instead of gallium. In particular, M is preferably one or more of gallium, aluminum, and yttrium.

For the sacrificial layer 110, an inorganic insulating material such as aluminum oxide, hafnium oxide, or silicon oxide can be used.

The sacrificial layer 110 is preferably formed using a material that can be dissolved in a solvent chemically stable with respect to at least the uppermost film of the EL layer 103B (the electron-transport layer 108B). Specifically, a material that will be dissolved in water or alcohol can be suitably used for the sacrificial layer 110. In deposition of the sacrificial layer 110, it is preferable that application of such a material dissolved in a solvent such as water or alcohol be performed by the aforementioned wet process and followed by heat treatment for evaporating the solvent. At this time, the heat treatment is preferably performed in a reduced-pressure atmosphere, in which case the solvent can be removed at a low temperature in a short time and thermal damage to the EL layer 103B can be reduced accordingly.

In the case where the sacrificial layer 110 having a stacked-layer structure is formed, the stacked-layer structure can include the first sacrificial layer formed using any of the above-described materials and the second sacrificial layer thereover.

The second sacrificial layer in that case is a film used as a hard mask for etching of the first sacrificial layer. In processing the second sacrificial layer, the first sacrificial layer is exposed. Thus, a combination of films having greatly different etching rates is selected for the first sacrificial layer and the second sacrificial layer. Thus, a film that can be used for the second sacrificial layer can be selected in accordance with the etching conditions of the first sacrificial layer and those of the second sacrificial layer.

For example, in the case where dry etching using a gas containing fluorine (also referred to as a fluorine-based gas) is performed for the etching of the second sacrificial layer, silicon, silicon nitride, silicon oxide, tungsten, titanium, molybdenum, tantalum, tantalum nitride, an alloy containing molybdenum and niobium, or an alloy containing molybdenum and tungsten can be used for the second sacrificial layer. Here, a metal oxide film such as IGZO or ITO is given as a film having high etching selectivity (that is, enabling low etching rate) in dry etching using the fluorine-based gas, and such a film can be used as the first sacrificial layer.

Note that the material for the second sacrificial layer is not limited to the above and can be selected from a variety of materials in accordance with the etching conditions of the first sacrificial layer and those of the second sacrificial layer. For example, any of the films that can be used for the first sacrificial layer can be used.

As the second sacrificial layer, a nitride film can be used, for example. Specifically, it is possible to use a nitride such as silicon nitride, aluminum nitride, hafnium nitride, titanium nitride, tantalum nitride, tungsten nitride, gallium nitride, or germanium nitride.

Alternatively, an oxide film can be used as the second sacrificial layer. A film of oxide or oxynitride such as silicon oxide, silicon oxynitride, aluminum oxide, aluminum oxynitride, hafnium oxide, or hafnium oxynitride can be typically used.

Then, the EL layer 103B over the electrode 551B is processed to have a predetermined shape as illustrated in FIG. 4B. For example, a sacrificial layer 110B is formed over the EL layer 103B; a resist is formed to have a desired shape over the sacrificial layer 110B by a photolithography method as a resist mask REG (see FIG. 4A); part of the sacrificial layer 110B not covered with the resist mask REG is removed by etching; the resist mask REG is removed; and part of the EL layer 103B not covered with the sacrificial layer 110B is then removed by etching, i.e., the EL layer 103B over the electrode 551G and the EL layer 103B over the electrode 551R are removed by etching, so that the EL layer 103B over the electrode 551B is processed to have side surfaces (or have their side surfaces exposed) or have a belt-like shape that extends in the direction intersecting the sheet of the diagram. Specifically, dry etching is performed using the patterned sacrificial layer 110B over the EL layer 103B overlapping with the electrode 551B (see FIG. 4B). Note that in the case where the sacrificial layer 110B has the aforementioned stacked-layer structure of the first sacrificial layer and the second sacrificial layer, the EL layer 103B may be processed into a predetermined shape in the following manner: part of the second sacrificial layer is etched with use of the resist mask REG, the resist mask REG is then removed, and part of the first sacrificial layer is etched with use of the second sacrificial layer as a mask. The partition 528 can be used as an etching stopper.

Next, as illustrated in FIG. 4C, with the sacrificial layer 110B remaining, the EL layer 103G (including the hole-injection/transport layer 104G, the light-emitting layer, and the electron-transport layer 108G) is formed over the sacrificial layer 110B, the electrode 551G, the electrode 551R, and the partition 528. For example, the EL layer 103G is formed by a vacuum evaporation method over the electrode 551G, the electrode 551R, and the partition 528 so as to cover them. Note that the EL layer 103G in FIG. 4C includes the hole-injection/transport layer 104G, the light-emitting layer, and the electron-transport layer 108G.

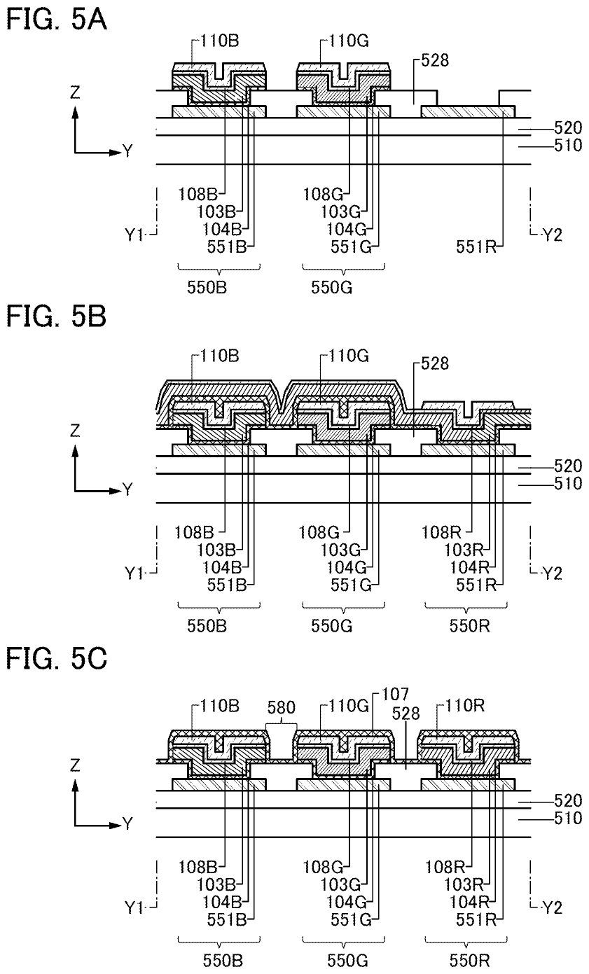

Then, the EL layer 103G over the electrode 551G is processed to have a predetermined shape as illustrated in FIG. 5A. For example, a sacrificial layer 110G is formed over the EL layer 103G; a resist is formed to have a desired shape over the sacrificial layer 110G by a photolithography method as a resist mask; part of the sacrificial layer 110G not covered with the resist mask is removed by etching; the resist mask is removed; and part of the EL layer 103G not covered with the sacrificial layer 110G is then removed by etching, i.e., the EL layer 103G over the electrode 551B and the EL layer 103G over the electrode 551R are removed by etching, so that the EL layer 103G over the electrode 551G is processed to have side surfaces (or have their side surfaces exposed) or have a belt-like shape that extends in the direction intersecting the sheet of the diagram. Specifically, dry etching is performed using the patterned sacrificial layer 110G over the EL layer 103G overlapping with the electrode 551G. Note that in the case where the sacrificial layer 110G has the aforementioned stacked-layer structure of the first sacrificial layer and the second sacrificial layer, the EL layer 103G may be processed into a predetermined shape in the following manner: part of the second sacrificial layer is etched with use of the resist mask, the resist mask is then removed, and part of the first sacrificial layer is etched with use of the second sacrificial layer as a mask. The partition 528 can be used as an etching stopper.

Next, as illustrated in FIG. 5B, with the sacrificial layer 110B and the sacrificial layer 110G respectively over the electron-transport layer 108B and the electron-transport layer 108G remaining, the EL layer 103R (including the hole-injection/transport layer 104R, the light-emitting layer, and the electron-transport layer 108R) is formed over the sacrificial layer 110B, the sacrificial layer 110G, the electrode 551R, and the partition 528. For example, the EL layer 103R is formed by a vacuum evaporation method over the sacrificial layer 110B, the sacrificial layer 110G, the electrode 551R, and the partition 528 so as to cover them. Note that in the EL layer 103R in FIG. 5B, the hole-injection/transport layer 104R, the light-emitting layer, and the electron-transport layer 108R are formed.

Then, the EL layer 103R over the electrode 551R is processed to have a predetermined shape as illustrated in FIG. 5C. For example, a sacrificial layer 110R is formed over the EL layer 103R; a resist is formed to have a desired shape over the sacrificial layer 110R by a photolithography method as a resist mask; part of the sacrificial layer 110 not covered with the resist mask is removed by etching; the resist mask is removed; and part of the EL layer 103R not covered with the sacrificial layer 110R is then removed by etching, i.e., the EL layer 103R over the electrode 551B and the EL layer 103R over the electrode 551G are removed by etching, so that the EL layer 103R over the electrode 551R is processed to have side surfaces (or have their side surfaces exposed) or have a belt-like shape that extends in the direction intersecting the sheet of the diagram. Specifically, dry etching is performed using the patterned sacrificial layer 110R over the EL layer 103R overlapping with the electrode 551R. Note that in the case where the sacrificial layer 110R has the aforementioned stacked-layer structure of the first sacrificial layer and the second sacrificial layer, the EL layer 103R may be processed into a predetermined shape in the following manner: part of the second sacrificial layer is etched with use of the resist mask, the resist mask is then removed, and part of the first sacrificial layer is etched with use of the second sacrificial layer as a mask. The partition 528 can be used as an etching stopper.

Then, an insulating layer 107 is formed over the sacrificial layers (110B, 110G, and 110R), the EL layers (103B, 103G, and 103R), and the partition 528. For example, the insulating layer 107 is formed by an ALD method over the sacrificial layers (110B, 110G, and 110R), the EL layers (103B, 103G, and 103R), and the partition 528 so as to cover them. In this case, the insulating layer 107 is formed in contact with the side surfaces of the EL layers (103B, 103G, and 103R) as illustrated in FIG. 5C. This can inhibit entry of oxygen, moisture, or constituent elements thereof into the inside through the side surfaces of the EL layers (103B, 103G, and 103R). For a material used for the insulating layer 107, aluminum oxide, magnesium oxide, hafnium oxide, gallium oxide, indium gallium zinc oxide, silicon nitride, silicon nitride oxide, or the like can be used, for example.

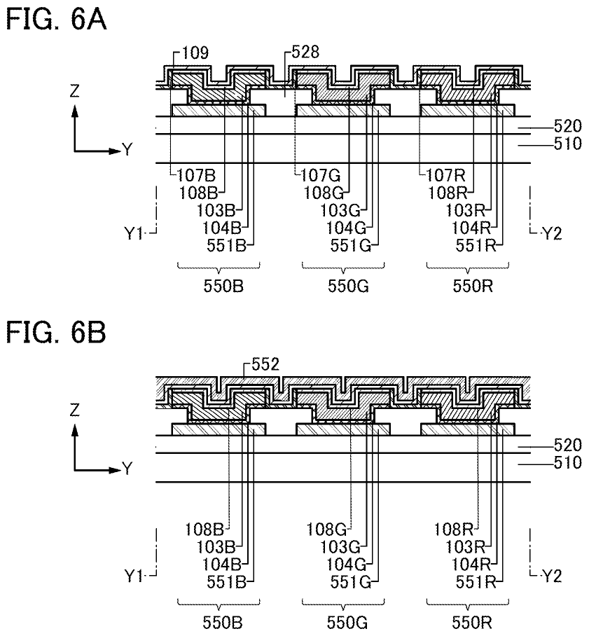

Next, as illustrated in FIG. 6A, the sacrificial layers (110B, 110G, and 110R) are removed, so that the insulating layers (107B, 107G, and 107R) are formed. Then, the electron-injection layer 109 is formed over the insulating layers (107B, 107G, and 107R) and the electron-transport layers (108B, 108G, and 108R). The electron-injection layer 109 is formed by a vacuum evaporation method, for example. Note that the electron-injection layer 109 is formed over the insulating layers (107B, 107G, 107R) and the electron-transport layers (108B, 108G, and 108R). The electron-injection layer 109 has a structure in contact with the EL layers (103B, 103G, and 103R) with the insulating layers (107B, 107G, and 107R) therebetween; not that the EL layers (103B, 103G, and 103R) illustrated in FIG. 6A include the hole-injection/transport layers (104R, 104G, and 104B), the light-emitting layers, and the electron-transport layers (108B, 108G, and 108R).

Next, as illustrated in FIG. 6B, the electrode 552 is formed. The electrode 552 is formed by a vacuum evaporation method, for example. The electrode 552 is formed over the electron-injection layer 109. The electrode 552 has a structure in contact with the side surfaces (or end portions) of the EL layers (103B, 103G, and 103R) with the electron-injection layer 109 and the insulating layers (107B, 107G, and 107R) therebetween; note that the EL layers (103B, 103G, and 103R) illustrated in FIG. 6B include the hole-injection/transport layers (104R, 104G, and 104B), the light-emitting layers, and the electron-transport layers (108B, 108G, and 108R). Thus, the EL layers (103B, 103G, and 103R) and the electrode 552, specifically the hole-injection/transport layers (104B, 104G, and 104R) in the EL layers (103B, 103G, and 103R) and the electrode 552 can be prevented from being electrically short-circuited.

Through the above steps, the EL layer 103B, the EL layer 103G, and the EL layer 103R in the light-emitting device 550B, the light-emitting device 550G, and the light-emitting device 550R can be processed to be separated from each other.

The EL layers (the EL layer 103B, the EL layer 103G, and the EL layer 103R) are processed to be separated by patterning using a photolithography method; hence, a high-resolution light-emitting apparatus (display panel) can be fabricated. End portions (side surfaces) of the EL layer processed by patterning using a photolithography method have substantially one surface (or are positioned on substantially the same plane).

In the EL layer, particularly the hole-injection layer, which is included in the hole-transport region between the anode and the light-emitting layer, often has high conductivity; thus, a hole-injection layer formed as a layer shared by adjacent light-emitting devices might cause crosstalk. Therefore, processing the EL layers to be separated by patterning using a photolithography method as shown in this structure example can suppress occurrence of crosstalk between adjacent light-emitting devices.

### Structure Example 2 of Light-Emitting Apparatus 700

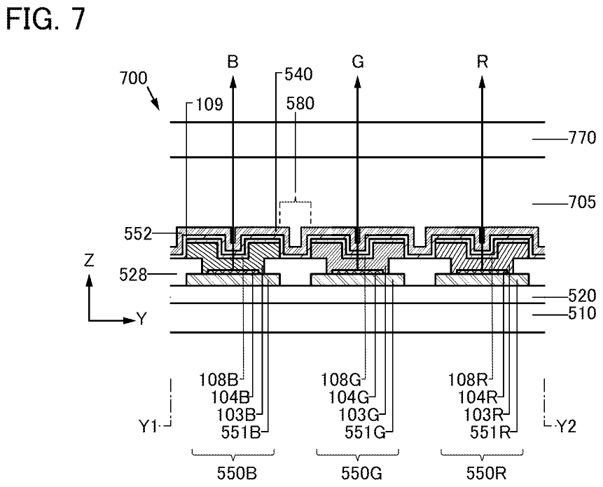

The light-emitting apparatus 700 illustrated in FIG. 7 includes the light-emitting device 550B, the light-emitting device 550G, the light-emitting device 550R, and the partition 528. The light-emitting device 550B, the light-emitting device 550G, the light-emitting device 550R, and the partition 528 are formed over the functional layer 520 provided over the first substrate 510. The functional layer 520 includes, for example, a gate line driver circuit GD, a source line driver circuit SD, and the like that are composed of a plurality of transistors, and wirings that electrically connect these circuits. The driver circuit GD and the driver circuit SD will be described in Embodiment 4. Note that these driver circuits are electrically connected to the light-emitting device 550B, the light-emitting device 550G, and the light-emitting device 550R, for example, to drive them.

The light-emitting device 550B, the light-emitting device 550G, and the light-emitting device 550R each have the device structure described in Embodiment 2. Specifically, the case is described in which the EL layer 103 in the structure illustrated in FIG. 1A differs between the light-emitting devices.

Note that specific structures of the light-emitting devices illustrated in FIG. 7 are the same as the structures of the light-emitting device 550B, the light-emitting device 550G, and the light-emitting device 550R described with reference to FIG. 2.

As illustrated in FIG. 7, the hole-injection/transport layers (104B, 104G, and 104R) in the EL layers (103B, 103G, and 103R) of the light-emitting devices (550B, 550G, and 550R) are smaller than the other functional layers in the EL layers and are covered with the functional layers stacked over the hole-injection/transport layers.

In this structure, the hole-injection/transport layers (104B, 104G, and 104R) in the EL layers are completely separated from each other by being covered with the other functional layers; thus, the insulating layers (107 in FIG. 2) for preventing a short circuit between the hole-injection/transport layers and the electrode 552, which are described in Structure example 1, are unnecessary.

The EL layers (103B, 103G, and 103R) in this structure are processed to be separated by patterning using a photolithography method; hence, end portions (side surfaces) of the processed EL layers have substantially one surface (or are positioned on substantially the same plane).

In the EL layer, particularly the hole-injection layer, which is included in the hole-transport region between the anode and the light-emitting layer, often has high conductivity; thus, a hole-injection layer formed as a layer shared by adjacent light-emitting devices might cause crosstalk. Therefore, processing the EL layers to be separated by patterning using a photolithography method as shown in this structure example can suppress occurrence of crosstalk between adjacent light-emitting devices.

### Structure Example 3 of Light-Emitting Apparatus 700

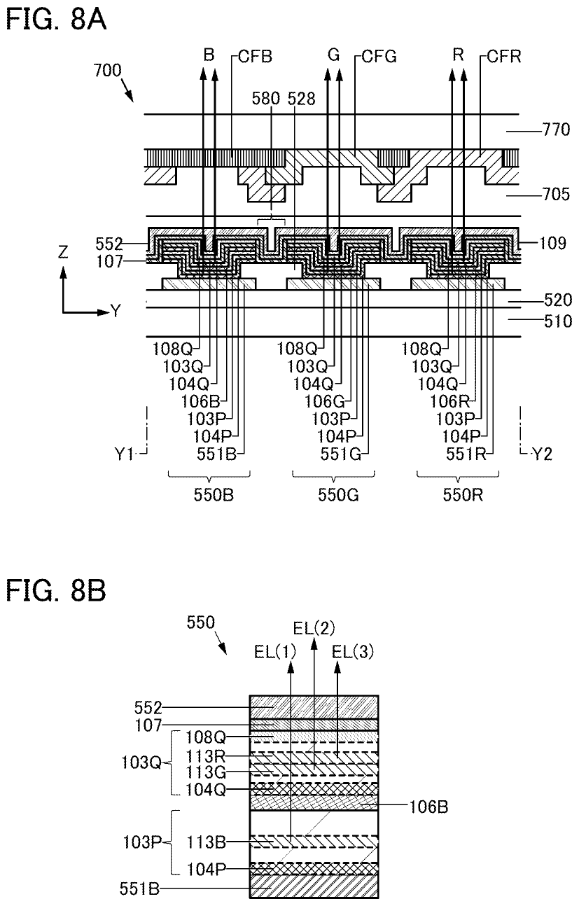

The light-emitting apparatus 700 illustrated in FIG. 8A includes the light-emitting device 550B, the light-emitting device 550G, the light-emitting device 550R, and the partition 528. The light-emitting device 550B, the light-emitting device 550G, the light-emitting device 550R, and the partition 528 are formed over the functional layer 520 provided over the first substrate 510. The functional layer 520 includes, for example, a gate line driver circuit GD, a source line driver circuit SD, and the like that are composed of a plurality of transistors, and wirings that electrically connect these circuits. The driver circuit GD and the driver circuit SD will be described in Embodiment 4. Note that these driver circuits are electrically connected to the light-emitting device 550B, the light-emitting device 550G, and the light-emitting device 550R, for example, to drive them.

The light-emitting device 550B, the light-emitting device 550G, and the light-emitting device 550R each have the device structure described in Embodiment 2. Specifically, the case is described in which the light-emitting devices share the EL layer 103 having the structure illustrated in FIG. 1B, i.e., a tandem structure.

The light-emitting device 550B has a stacked-layer structure illustrated in FIG. 8A, which includes the electrode 551B, the electrode 552, EL layers (103P and 103Q), a charge-generation layer 106B, the electron-transport layer 108B, and the insulating layer 107. Note that a specific structure of each layer is as described in Embodiment 2. The electrode 551B and the electrode 552 overlap each other. The EL layer 103P and the EL layer 103Q are stacked with the charge-generation layer 106B therebetween, and the EL layer 103P, the EL layer 103Q, and the charge-generation layer 106B are positioned between the electrode 551B and the electrode 552. Note that each of the EL layers 103P and 103Q has a stacked-layer structure of layers having different functions, including a light-emitting layer, like the EL layers 103, 103a, 103b, and 103c described in Embodiment 2. The EL layer 103P is capable of emitting blue light, for example, and the EL layer 103Q is capable of emitting yellow light, for example.

In FIG. 8A, only a hole-injection/transport layer 104P is illustrated as layers included in the EL layer 103P, and only a hole-injection/transport layer 104Q, an electron-transport layer 108Q, and the electron-injection layer 109 are illustrated as layers included in the EL layer 103Q. Thus, in the following description, the term “EL layer” (the EL layer 103P and the EL layer 103Q) is used for convenience to describe the layers included in the EL layer as well. The electron-transport layer may have a stacked-layer structure, and may include a hole-blocking layer for blocking holes that move from the anode side to the cathode side through the light-emitting layer. The electron-injection layer 109 may have a stacked-layer structure in which some or all of layers are formed using different materials.

The insulating layer 107 is formed while a sacrificial layer formed over some layers of the EL layer 103Q (in this embodiment, the layers up to the electron-transport layer 108Q over the light-emitting layer) remains over the electrode 551B as illustrated in FIG. 8A. Thus, the insulating layer 107 is formed in contact with the side surfaces (or the end portions) of the EL layer 103P, part (the above) of the EL layer 103Q, and the charge-generation layer 106B. Accordingly, it is possible to inhibit entry of oxygen, moisture, or constituent elements thereof into the inside through the side surfaces of the EL layer 103P, the EL layer 103Q, and the charge-generation layer 106B. For the insulating layer 107, aluminum oxide, magnesium oxide, hafnium oxide, gallium oxide, indium gallium zinc oxide, silicon nitride, silicon nitride oxide, or the like can be used, for example. The insulating layer 107 can be formed by a sputtering method, a CVD method, an MBE method, a PLD method, an ALD method, or the like and is formed preferably by an ALD method, which achieves favorable coverage.

The electron-injection layer 109 is formed to cover part of the EL layer 103Q (the electron-transport layer 108Q) and the insulating layer 107. The electron-injection layer 109 preferably has a stacked-layer structure of two or more layers having different electric resistances. For example, the electron-injection layer 109 may have one of the following structures: a structure in which a first layer in contact with the electron-transport layer 108Q is formed using only an electron-transport material, and a second layer formed using an electron-transport material containing a metal material is stacked over the first layer; or the aforementioned structure including a third layer formed using an electron-transport material containing a metal material, between the first layer and the electron-transport layer 108Q.

The electrode 552 is formed over the electron-injection layer 109. Note that the electrode 551B and the electrode 552 have an overlap region. The EL layer 103P, the EL layer 103Q, and the charge-generation layer 106B are positioned between the electrode 551B and the electrode 552. Thus, the electron-injection layer 109 has a structure in contact with the side surfaces (or end portions) of the EL layer 103Q, the EL layer 103P, and the charge-generation layer 106B with the insulating layer 107 therebetween, or the electrode 552 has a structure in contact with the side surfaces (or end portions) of the EL layer 103Q, the EL layer 103P, and the charge-generation layer 106B with the electron-injection layer 109 and the insulating layer 107 therebetween. Consequently, the EL layer 103P and the electrode 552, specifically the hole-injection/transport layer 104P in the EL layer 103P and the electrode 552 or the EL layer 103Q and the electrode 552, more specifically the hole-injection/transport layer 104Q in the EL layer 103Q and the electrode 552 or the charge-generation layer 106B and the electrode 552 can be prevented from being electrically short-circuited.

The light-emitting device 550G has a stacked-layer structure illustrated in FIG. 8A, which includes the electrode 551G, the electrode 552, the EL layers (103P and 103Q), a charge-generation layer 106G, the electron-transport layer 108G, and the insulating layer 107. Note that a specific structure of each layer is as described in Embodiment 1. The electrode 551G and the electrode 552 overlap each other. The EL layer 103P and the EL layer 103Q are stacked with the charge-generation layer 106G therebetween, and the EL layer 103P, the EL layer 103Q, and the charge-generation layer 106G are positioned between the electrode 551G and the electrode 552.

The insulating layer 107 is formed while a sacrificial layer formed over some layers of the EL layer 103Q (in this embodiment, the layers up to the electron-transport layer 108Q over the light-emitting layer) remains over the electrode 551G as illustrated in FIG. 8A. Thus, the insulating layer 107 is formed in contact with the side surfaces (or the end portions) of the EL layer 103P, part (the above) of the EL layer 103Q, and the charge-generation layer 106B. Accordingly, it is possible to inhibit entry of oxygen, moisture, or constituent elements thereof into the inside through the side surfaces of the EL layer 103P, the EL layer 103Q, and the charge-generation layer 106G. For the insulating layer 107, aluminum oxide, magnesium oxide, hafnium oxide, gallium oxide, indium gallium zinc oxide, silicon nitride, silicon nitride oxide, or the like can be used, for example. The insulating layer 107 can be formed by a sputtering method, a CVD method, an MBE method, a PLD method, an ALD method, or the like and is formed preferably by an ALD method, which achieves favorable coverage.

The electron-injection layer 109 is formed to cover part of the EL layer 103Q (the electron-transport layer 108Q) and the insulating layer 107. The electron-injection layer 109 preferably has a stacked-layer structure of two or more layers having different electric resistances. For example, the electron-injection layer 109 may have one of the following structures: a structure in which a first layer in contact with the electron-transport layer 108Q is formed using only an electron-transport material, and a second layer formed using an electron-transport material containing a metal material is stacked over the first layer; or the aforementioned structure including a third layer formed using an electron-transport material containing a metal material, between the first layer and the electron-transport layer 108Q.

The electrode 552 is formed over the electron-injection layer 109. Note that the electrode 551G and the electrode 552 have an overlap region. The EL layer 103P, the EL layer 103Q, and the charge-generation layer 106G are positioned between the electrode 551G and the electrode 552. Thus, the electron-injection layer 109 has a structure in contact with the side surfaces (or end portions) of the EL layer 103Q, the EL layer 103P, and the charge-generation layer 106G with the insulating layer 107 therebetween, or the electrode 552 has a structure in contact with the side surfaces (or end portions) of the EL layer 103Q, the EL layer 103P, and the charge-generation layer 106G with the electron-injection layer 109 and the insulating layer 107 therebetween. Consequently, the EL layer 103P and the electrode 552, specifically the hole-injection/transport layer 104P in the EL layer 103P and the electrode 552 or the EL layer 103Q and the electrode 552, more specifically the hole-injection/transport layer 104Q in the EL layer 103Q and the electrode 552 or the charge-generation layer 106G and the electrode 552 can be prevented from being electrically short-circuited.

The light-emitting device 550R has a stacked-layer structure illustrated in FIG. 8A, which includes the electrode 551R, the electrode 552, the EL layers (103P and 103Q), a charge-generation layer 106R, the electron-transport layer 108R, and the insulating layer 107. Note that a specific structure of each layer is as described in Embodiment 1. The electrode 551R and the electrode 552 overlap each other. The EL layer 103P and the EL layer 103Q are stacked with the charge-generation layer 106R therebetween, and the EL layer 103P, the EL layer 103Q, and the charge-generation layer 106R are positioned between the electrode 551R and the electrode 552.

The insulating layer 107 is formed while a sacrificial layer formed over some layers of the EL layer 103Q (in this embodiment, the layers up to the electron-transport layer 108Q over the light-emitting layer) remains over the electrode 551R as illustrated in FIG. 8A. Thus, the insulating layer 107 is formed in contact with the side surfaces (or the end portions) of the EL layer 103P, part (the above) of the EL layer 103Q, and the charge-generation layer 106R. Accordingly, it is possible to inhibit entry of oxygen, moisture, or constituent elements thereof into the inside through the side surfaces of the EL layer 103P, the EL layer 103Q, and the charge-generation layer 106R. For the insulating layer 107, aluminum oxide, magnesium oxide, hafnium oxide, gallium oxide, indium gallium zinc oxide, silicon nitride, silicon nitride oxide, or the like can be used, for example. The insulating layer 107 can be formed by a sputtering method, a CVD method, an MBE method, a PLD method, an ALD method, or the like and is formed preferably by an ALD method, which achieves favorable coverage.

The electron-injection layer 109 is formed to cover part of the EL layer 103Q (the electron-transport layer 108Q) and the insulating layer 107. The electron-injection layer 109 preferably has a stacked-layer structure of two or more layers having different electric resistances. For example, the electron-injection layer 109 may have one of the following structures: a structure in which a first layer in contact with the electron-transport layer 108Q is formed using only an electron-transport material, and a second layer formed using an electron-transport material containing a metal material is stacked over the first layer; or the aforementioned structure including a third layer formed using an electron-transport material containing a metal material, between the first layer and the electron-transport layer 108Q.

The electrode 552 is formed over the electron-injection layer 109. Note that the electrode 551R and the electrode 552 have an overlap region. The EL layer 103P, the EL layer 103Q, and the charge-generation layer 106R are positioned between the electrode 551R and the electrode 552. Thus, the electron-injection layer 109 has a structure in contact with the side surfaces (or end portions) of the EL layer 103Q, the EL layer 103P, and the charge-generation layer 106R with the insulating layer 107 therebetween, or the electrode 552 has a structure in contact with the side surfaces (or end portions) of the EL layer 103Q, the EL layer 103P, and the charge-generation layer 106R with the electron-injection layer 109 and the insulating layer 107 therebetween. Consequently, the EL layer 103P and the electrode 552, specifically the hole-injection/transport layer 104P in the EL layer 103P and the electrode 552 or the EL layer 103Q and the electrode 552, more specifically the hole-injection/transport layer 104Q in the EL layer 103Q and the electrode 552 or the charge-generation layer 106R and the electrode 552 can be prevented from being electrically short-circuited.

The EL layers (103P and 103Q) and the charge-generation layer 106R included in the light-emitting devices are processed to be separated between the light-emitting devices by patterning using a photolithography method; thus, the end portions (side surfaces) of the processed EL layers have substantially one surface (or are positioned on substantially the same plane).

The EL layers (103P and 103Q) and the charge-generation layer 106R are provided with the space 580 between one light-emitting device and the adjacent light-emitting device. The charge-generation layer 106R and the hole-injection layers included in the hole-transport regions in the EL layers (103P and 103Q) often have high conductivity; therefore, these layers formed as layers shared by adjacent light-emitting devices might cause crosstalk. Thus, providing the space 580 as shown in this structure example can suppress occurrence of crosstalk between adjacent light-emitting devices.

When electrical continuity is established between the EL layers in the light-emitting device 550R, the light-emitting device 550G, and the light-emitting device 550R in a light-emitting apparatus (display panel) with a high resolution exceeding 1000 ppi, a crosstalk phenomenon occurs, resulting in a narrower color gamut that the light-emitting apparatus is capable of reproducing. Providing the space 580 in a high-resolution display panel with more than 1000 ppi, preferably more than 2000 ppi, or further preferably in an ultrahigh-resolution display panel with more than 5000 ppi allows the display panel to express vivid colors.

In this structure example, the light-emitting device 550B, the light-emitting device 550G, and the light-emitting device 550R each emit white light. Accordingly, the second substrate 770 includes a coloring layer CFB, a coloring layer CFG, and a coloring layer CFR. Note that these coloring layers may be provided to partly overlap with each other as illustrated in FIG. 8A. When the coloring layers partly overlap with each other, the overlap portion can function as a light-blocking film. In this structure example, a material that preferentially transmits blue light (B) is used for the coloring layer CFB, a material that preferentially transmits green light (G) is used for the coloring layer CFG, and a material that preferentially transmits red light (R) is used for the coloring layer CFR, for example.

FIG. 8B illustrates a structure of the light-emitting device 550B in the case where each of the light-emitting device 550B, the light-emitting device 550G, and the light-emitting device 550R (illustrated as a light-emitting device 550 collectively) is a white-light-emitting device. The EL layer 103P and the EL layer 103Q are stacked over the electrode 551B, with the charge-generation layer 106B between the EL layers. The EL layer 103P includes the light-emitting layer 113B that emits blue light EL(1), and the EL layer 103Q includes the light-emitting layer 113G that emits green light EL(2) and the light-emitting layer 113R that emits red light EL(3).

Note that a color conversion layer can be used instead of the coloring layer. For example, nanoparticles, quantum dots, or the like can be used for the color conversion layer.

For example, a color conversion layer that converts blue light into green light can be used instead of the coloring layer CFG. Thus, blue light emitted from the light-emitting device 550G can be converted into green light. Moreover, a color conversion layer that converts blue light into red light can be used instead of the coloring layer CFR. Thus, blue light emitted from the light-emitting device 550R can be converted into red light.

### Structure Example 4 of Light-Emitting Apparatus 700

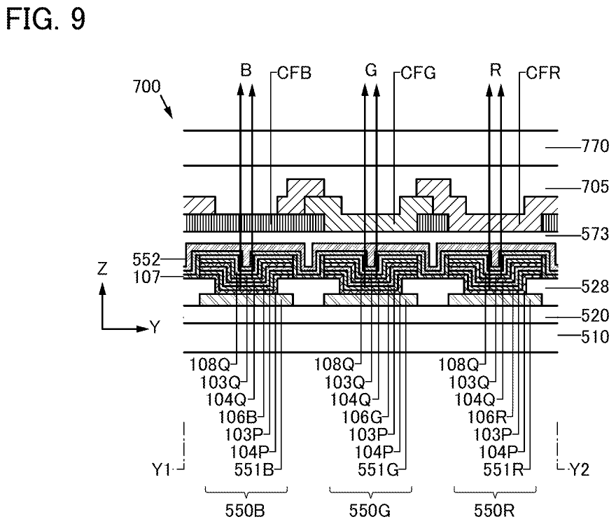

The light-emitting apparatus (display panel) 700 illustrated in FIG. 9 includes the light-emitting device 550B, the light-emitting device 550G, the light-emitting device 550R, and the partition 528. The light-emitting device 550B, the light-emitting device 550G, the light-emitting device 550R, and the partition 528 are formed over the functional layer 520 provided over the first substrate 510. The functional layer 520 includes, for example, a gate line driver circuit GD, a source line driver circuit SD, and the like that are composed of a plurality of transistors, and wirings that electrically connect these circuits. The driver circuit GD and the driver circuit SD will be described in Embodiment 4. Note that these driver circuits are electrically connected to the light-emitting device 550B, the light-emitting device 550G, and the light-emitting device 550R, for example, to drive them.

The light-emitting device 550B, the light-emitting device 550G, and the light-emitting device 550R each have the device structure described in Embodiment 1. Specifically, such a structure is suitable for the case in which the light-emitting devices share the EL layer 103 having the structure illustrated in FIG. 1B, i.e., a tandem structure.

Note that specific structures of the light-emitting devices illustrated in FIG. 9 are the same as the structures of the light-emitting device 550B, the light-emitting device 550G, and the light-emitting device 550R described with reference to FIG. 8B, and each of the light-emitting devices emits white light.

The light-emitting apparatus in this structure example is different from the light-emitting apparatus illustrated in FIG. 8A in including the coloring layer CFB, the coloring layer CFG, and the coloring layer CFR formed over the light-emitting devices over the first substrate 510.

In other words, a first insulating layer 573 is provided over the electrode 552 of each light-emitting device formed over the first substrate 510, and the coloring layer CFB, the coloring layer CFG, and the coloring layer CFR are provided over the first insulating layer 573.

A second insulating layer 705 is provided over the coloring layer CFB, the coloring layer CFG, and the coloring layer CFR. The second insulating layer 705 includes a region sandwiched between the second substrate 770 and the first substrate 510 on the side closer to the coloring layers (CFB, CFG, and CFR), which is provided with the functional layer 520, the light-emitting devices (550B, 550G, and 550R), and the coloring layers CFB, CFG, and CFR. The second insulating layer 705 has a function of attaching the first substrate 510 and the second substrate 770.

For the first insulating layer 573 and the second insulating layer 705, an inorganic material, an organic material, a composite material of an inorganic material and an organic material, or the like can be used.

Specifically, an inorganic oxide film, an inorganic nitride film, an inorganic oxynitride film, or the like, or a stacked-layer material in which a plurality of films selected from these films are stacked can be used as the inorganic material. For example, a film including a silicon oxide film, a silicon nitride film, a silicon oxynitride film, an aluminum oxide film, or the like, or a film including a stacked-layer material in which a plurality of films selected from these films are stacked can be used. Note that the silicon nitride film is a dense film and has an excellent function of inhibiting diffusion of impurities. Alternatively, for an oxide semiconductor (e.g., an IGZO film), a stacked-layer structure of an aluminum oxide film and an IGZO film over the aluminum oxide film, for example, can be used.

For example, polyester, polyolefin, polyamide, polyimide, polycarbonate, polysiloxane, an acrylic resin, or the like, or a stacked-layer material, a composite material, or the like of a plurality of resins selected from these resins can be used for the organic material. Alternatively, an organic material such as a reactive curable adhesive, a photocurable adhesive, a thermosetting adhesive, or/and an anaerobic adhesive can be used.

### Manufacturing Method Example 1 of Light-Emitting Apparatus

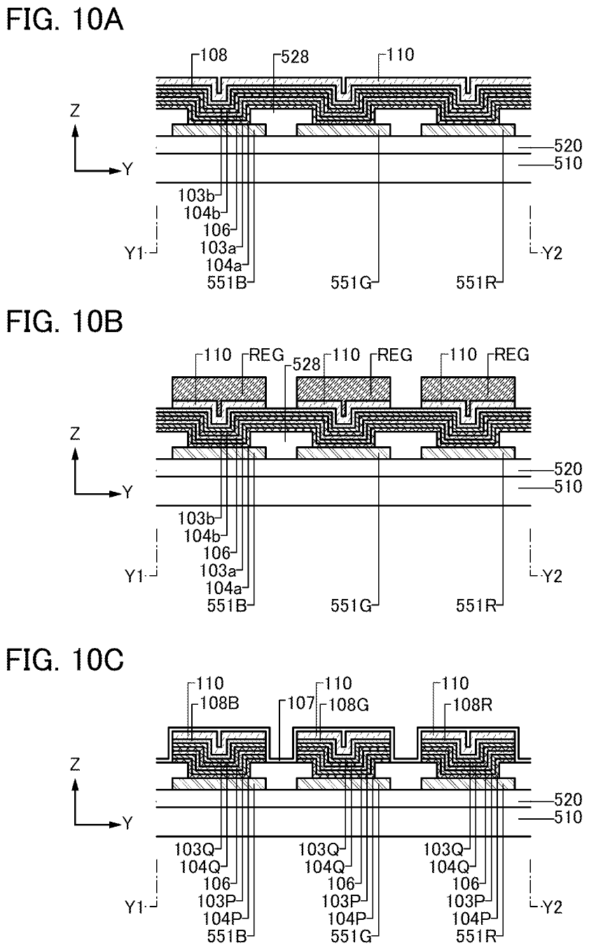

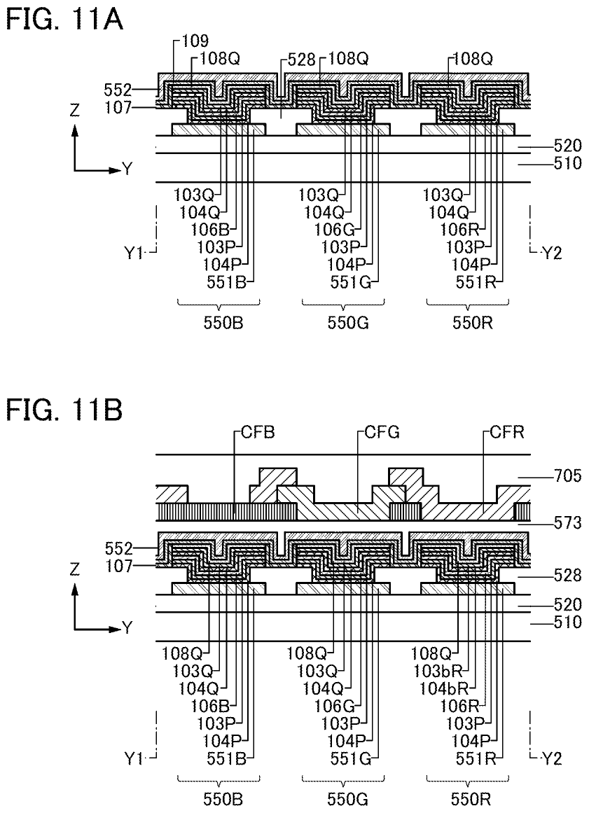

Next, a method for manufacturing the light-emitting apparatus illustrated in FIG. 9 is described with reference to FIG. 10A to FIG. 11B.

As illustrated in FIG. 10A, over the electrodes (551B, 551G, and 551R) and the partition 528 (see FIG. 3B) formed over the first substrate 510, the EL layer 103a (including the hole-injection/transport layer 104a), the charge-generation layers (106R, 106G, and 106B), and the EL layer 103b (including the hole-injection/transport layer 104b and the electron-transport layer 108) are formed so as to cover them. Furthermore, the sacrificial layer 110 is formed over the EL layer 103b. Description of the structure of the sacrificial layer 110 is not made because the structure is similar to that described with reference to FIG. 4A.

Then, as illustrated in FIG. 10B, the resist masks REG are formed in the following manner: a resist is applied onto the sacrificial layer 110, and the resist in the regions of the sacrificial layer 110 which do not overlap with the electrode 551B, the electrode 551G, or the electrode 551R is removed, whereby the resist remains in the regions of the sacrificial layer 110 which overlap with the electrode 551B, the electrode 551G, and the electrode 551R. For example, the resist applied onto the sacrificial layer 110 is formed into desired shapes by a photolithography method. Then, portions of the sacrificial layer 110 not covered with the thus formed resist masks REG are removed by etching. After that, the resist masks REG are removed, and portions of the EL layer 103b (including the hole-injection/transport layer 104b and the electron-transport layer 108), portions of the charge-generation layer 106, and portions of the EL layer 103b (including the hole-injection/transport layer 104b and the electron-transport layer 108) which are not covered with the sacrificial layers are removed by etching, whereby the EL layer 103b, the charge-generation layer 106, and the EL layer 103b are processed to have side surfaces (or have their side surfaces exposed) or have a belt-like shape that extends in the direction intersecting the sheet of the diagram. Specifically, dry etching is performed with use of the sacrificial layers 110 formed in patterns over the EL layer 103b (including the hole-injection/transport layer 104b and the electron-transport layer 108) (see FIG. 10C). Although not shown in FIG. 10C, in the case where the sacrificial layers 110 each have the stacked-layer structure of the first sacrificial layer and the second sacrificial layer, the EL layer 103Q (including the hole-injection/transport layer 104Q and the electron-transport layer 108), the charge-generation layer 106, and the EL layer 103P (including the hole-injection/transport layer 104P) may be processed into a predetermined shape in the following manner as in the description with reference to FIG. 4A: part of the second sacrificial layer is etched with use of the resist mask, the resist mask is then removed, and part of the first sacrificial layer is etched with use of the second sacrificial layer as a mask. The partition 528 can be used as an etching stopper.

Then, the insulating layer 107 is formed over the sacrificial layers 110, the EL layers (103P and 103Q), and the partition 528. For example, the insulating layer 107 is formed by an ALD method over the sacrificial layers 110, the EL layers (103P and 103Q), and the partition 528 so as to cover them. In this case, the insulating layer 107 is formed in contact with the side surfaces of the EL layers (103P and 103Q) as illustrated in FIG. 10C. Specifically, the insulating layer 107 is formed on side surfaces that are exposed when the EL layer 103P (including the hole-injection/transport layer 104P), the charge-generation layers (106B, 106G, and 106R), and the EL layer 103Q (including the hole-injection/transport layer 104Q and the electron-transport layer 108Q) are processed by etching. This can inhibit entry of oxygen, moisture, or constituent elements thereof into the inside through the side surfaces of the EL layers (103P and 103Q). As a material used for the insulating layer 107, aluminum oxide, magnesium oxide, hafnium oxide, gallium oxide, indium gallium zinc oxide, silicon nitride, silicon nitride oxide, or the like can be used, for example. As a material used for the insulating layer 107, the hole-transport material described in Embodiment 1 can be used.

Then, as illustrated in FIG. 11A, the sacrificial layers 110 are removed, and the electron-injection layer 109 is formed over the insulating layer 107 and the electron-transport layer (108Q). The electron-injection layer 109 is formed by a vacuum evaporation method, for example. Note that the electron-injection layer 109 is formed over the insulating layer 107 and the electron-transport layer (108Q). The electron-injection layer 109 has a structure in contact with the side surfaces of the EL layers (103P and 103Q) and the charge-generation layers (106B, 106G, and 106R) with the insulating layers 107 therebetween; note that the EL layers (103P and 103Q) illustrated in FIG. 11A includes the hole-injection/transport layers (104P and 104Q), the light-emitting layers, and the electron-transport layers (108Q).

Next, the electrode 552 is formed over the electron-injection layer 109. The electrode 552 is formed by a vacuum evaporation method, for example. The electrode 552 has a structure in contact with the side surfaces (or end portions) of the EL layers (103P and 103Q) and the charge-generation layers (106B, 106G, and 106R) with the electron-injection layer 109 and the insulating layers 107 therebetween; note that the EL layers (103P and 103Q) illustrated in FIG. 11A includes the hole-injection/transport layers (104P and 104Q), the light-emitting layers, and the electron-transport layers (108Q). Thus, the EL layers (103P and 103Q) and the electrode 552, specifically the hole-injection/transport layers (104P and 104Q) in the EL layers (103P and 103Q) and the electrode 552 can be prevented from being electrically short-circuited.

In the above manner, the EL layers 103P (each including the hole-injection/transport layer 104P), the charge-generation layers (106B, 106G, and 106R), and the EL layers 103Q (each including the hole-injection/transport layer 104Q and the electron-transport layer 108Q) can be separately formed between the light-emitting device 550B, the light-emitting device 550G, and the light-emitting device 550R by one patterning using a photolithography method.

Next, the insulating layer 573, the coloring layer CFB, the coloring layer CFG, the coloring layer CFR, and the insulating layer 705 are formed (see FIG. 11B).

For example, the insulating layer 573 is formed by stacking a flat film and a dense film. Specifically, the flat film is formed by a coating method, and the dense film is stacked over the flat film by a chemical vapor deposition method, an atomic layer deposition (ALD) method, or the like. Thus, the insulating layer 573 with high quality and less defects can be formed.

For example, with use of a color resist, the coloring layer CFB, the coloring layer CFG, and the coloring layer CFR are formed into a predetermined shape. Note that the coloring layers are processed so that the coloring layer CFR and the coloring layer CFB overlap with each other over the partition 528. Thus, a phenomenon of entrance of light emitted from an adjacent light-emitting device can be inhibited.

An inorganic material, an organic material, a composite material of an inorganic material and an organic material, or the like can be used for the insulating layer 705.

The EL layers (103P and 103Q) and the charge-generation layer 106R included in the light-emitting devices are processed to be separated between the light-emitting devices by patterning using a photolithography method; thus, a high-resolution light-emitting apparatus (display panel) can be fabricated. End portions (side surfaces) of the EL layer processed by patterning using a photolithography method have substantially one surface (or are positioned on substantially the same plane).

The charge-generation layers (106B, 106G, and 106R) and the hole-injection layers included in the hole-transport regions in the EL layers (103P and 103Q) often have high conductivity; therefore, these layers formed as layers shared by adjacent light-emitting devices might cause crosstalk. Therefore, processing the EL layers to be separated by patterning using a photolithography method as shown in this structure example can suppress occurrence of crosstalk between adjacent light-emitting devices.

### Structure Example 5 of Light-Emitting Apparatus 700

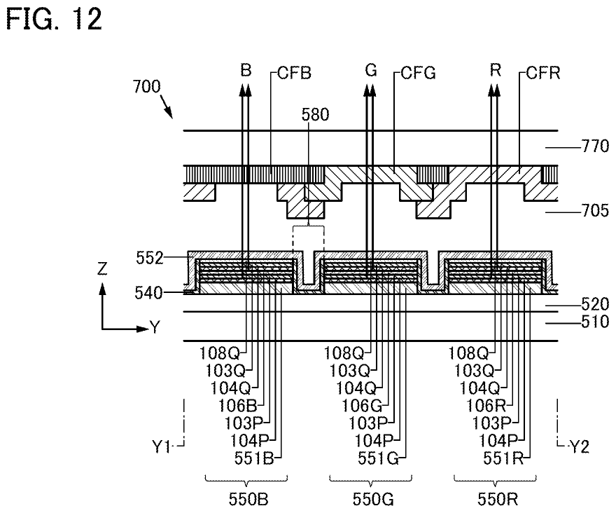

The light-emitting apparatus (display panel) 700 illustrated in FIG. 12 includes the light-emitting device 550B, the light-emitting device 550G, the light-emitting device 550R, and the partition 528. The light-emitting device 550B, the light-emitting device 550G, the light-emitting device 550R, and the partition 528 are formed over the functional layer 520 provided over the first substrate 510. The functional layer 520 includes, for example, a gate line driver circuit GD, a source line driver circuit SD, and the like that are composed of a plurality of transistors, and wirings that electrically connect these circuits. The driver circuit GD and the driver circuit SD will be described in Embodiment 4. Note that these driver circuits are electrically connected to the light-emitting device 550B, the light-emitting device 550G, and the light-emitting device 550R, for example, to drive them.

The light-emitting device 550B, the light-emitting device 550G, and the light-emitting device 550R each have the device structure described in Embodiment 2. Specifically, such a device is suitable for the case in which the light-emitting devices share the EL layer 103 having the structure illustrated in FIG. 1B, i.e., a tandem structure.

As illustrated in FIG. 12, the space 580 is provided between the light-emitting devices, for example, between the light-emitting device 550B and the light-emitting device 550G. An insulating layer 540 is formed in the space 580.

For example, the insulating layer 540 can be formed in the space 580 over the partition 528 by a photolithography method after the EL layer 103P (including the hole-injection/transport layer 104P), the charge-generation layers (106B, 106G, and 106R), and the EL layer 103Q (including the hole-injection/transport layer 104Q) are separately formed between the light-emitting devices by patterning using a photolithography method. Furthermore, the electrode 552 can be formed over the EL layer 103Q (including the hole-injection/transport layer 104Q) and the insulating layer 540.

In this structure, the EL layers are separated from each other by the insulating layer 540; thus, the insulating layer described in Structure example 3 (the insulating layer 107 in FIG. 8A and FIG. 8B) is unnecessary.

The EL layers (103P and 103Q) and the charge-generation layer 106R included in the light-emitting devices are processed to be separated between the light-emitting devices by patterning using a photolithography method; thus, a high-resolution light-emitting apparatus (display panel) can be fabricated. End portions (side surfaces) of the EL layer processed by patterning using a photolithography method have substantially one surface (or are positioned on substantially the same plane).

The charge-generation layers (106B, 106G, and 106R) and the hole-injection layers included in the hole-transport regions in the EL layers (103P and 103Q) often have high conductivity; therefore, these layers formed as layers shared by adjacent light-emitting devices might cause crosstalk. Therefore, processing the EL layers to be separated by patterning using a photolithography method as shown in this structure example can suppress occurrence of crosstalk between adjacent light-emitting devices.

The structure described in this embodiment can be used in an appropriate combination with any of the structures described in the other embodiments.

### Embodiment 4

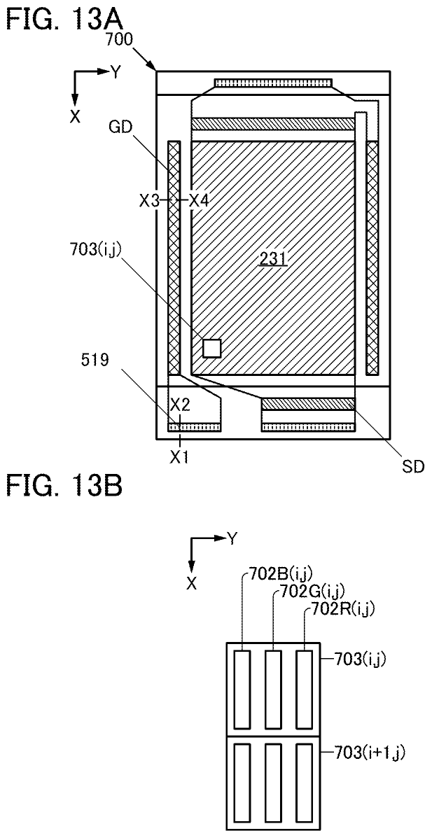

In this embodiment, a light-emitting apparatus of one embodiment of the present invention will be described with reference to FIG. 13A to FIG. 15B. The light-emitting apparatus 700 illustrated in FIG. 13A to FIG. 15B includes the light-emitting device described in Embodiment 2. The light-emitting apparatus 700 described in this embodiment can be referred to as a display panel because it can be used in a display portion of an electronic device and the like.

As illustrated in FIG. 13A, the light-emitting apparatus 700 described in this embodiment includes a display region 231, and the display region 231 includes a pixel set 703(i,j) (i is an integer greater than or equal to 1 and j is an integer greater than or equal to 1). A pixel set 703(i+1,j) adjacent to the pixel set 703(i,j) is provided as illustrated in FIG. 13B.

Note that a plurality of pixels can be used in the pixel 703(0. For example, a plurality of pixels capable of displaying colors with different hues can be used. Note that the plurality of pixels can be referred to as subpixels. A set of subpixels can be referred to as a pixel.

This enables additive mixture or subtractive mixture of colors displayed by the plurality of pixels. It is possible to display a color of a hue that an individual pixel cannot display.

Specifically, a pixel 702B(i,j) displaying blue, a pixel 702G(i,j) displaying green, and a pixel 702R(i,j) displaying red can be used in the pixel 703(i,j). The pixel 702B(i,j), the pixel 702G(i,j), and the pixel 702R(i,j) can each be referred to as a subpixel.

A pixel displaying white or the like may be used in addition to the above set in the pixel 703(i,j), for example. A pixel displaying cyan, a pixel displaying magenta, and a pixel displaying yellow may be used in the pixel 703(i,j) as subpixels.

A pixel that emits infrared light in addition to the above set may be used in the pixel 703(i,j). Specifically, a pixel that emits light including light with a wavelength greater than or equal to 650 nm and less than or equal to 1000 nm can be used in the pixel 703(i,j).

The light-emitting apparatus 700 includes the driver circuit GD and the driver circuit SD around the display region 231 in FIG. 13A. The light-emitting apparatus 700 also includes a terminal 519 electrically connected to the driver circuit GD, the driver circuit SD, and the like. The terminal 519 can be electrically connected to a flexible printed circuit FPC1, for example.

The driver circuit GD has a function of supplying a first selection signal and a second selection signal. For example, the driver circuit GD is electrically connected to a conductive film G1(i) described later to supply the first selection signal, and is electrically connected to a conductive film G2(i) described later to supply the second selection signal. The driver circuit SD has a function of supplying an image signal and a control signal, and the control signal includes a first level and a second level. For example, the driver circuit SD is electrically connected to a conductive film S1g(j) described later to supply the image signal, and is electrically connected to a conductive film S2g(j) described later to supply the control signal.

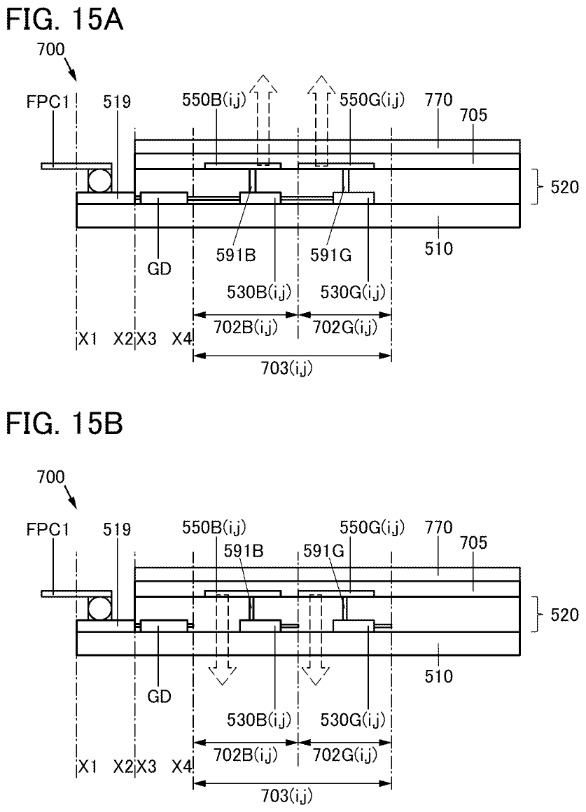

FIG. 15A shows a cross-sectional view of the light-emitting apparatus taken along each of the dashed-dotted line X1-X2 and the dashed-dotted line X3-X4 in FIG. 13A. As illustrated in FIG. 15A, the light-emitting apparatus 700 includes the functional layer 520 between the first substrate 510 and the second substrate 770. The functional layer 520 includes, for example, the driver circuit GD, the driver circuit SD, and the like that are described above and wirings that electrically connect these circuits. The structure of the functional layer 520 illustrated in FIG. 15A includes a pixel circuit 530B(i,j), a pixel circuit 530G(i,j), and the driver circuit GD; however, it is not limited thereto.

Each pixel circuit (e.g., the pixel circuit 530B(i,j) and the pixel circuit 530G(i,j) in FIG. 15A) included in the functional layer 520 is electrically connected to light-emitting devices (e.g., a light-emitting device 550B(i,j) and a light-emitting device 550G(i,j) in FIG. 15A) formed over the functional layer 520. Specifically, the light-emitting device 550B(i,j) is electrically connected to the pixel circuit 530B(i,j) through an opening 591B, and the light-emitting device 550G(i,j) is electrically connected to the pixel circuit 530G(i,j) through an opening 591G. The light-emitting apparatus 700 includes an insulating layer 705 over the functional layer 520 and the light-emitting devices, and the insulating layer 705 has a function of attaching a second substrate 770 and the functional layer 520.

As the second substrate 770, a substrate where touch sensors are arranged in a matrix can be used. For example, a substrate provided with a capacitive touch sensor or an optical touch sensor can be used for the second substrate 770. Thus, the light-emitting apparatus of one embodiment of the present invention can be used as a touch panel.

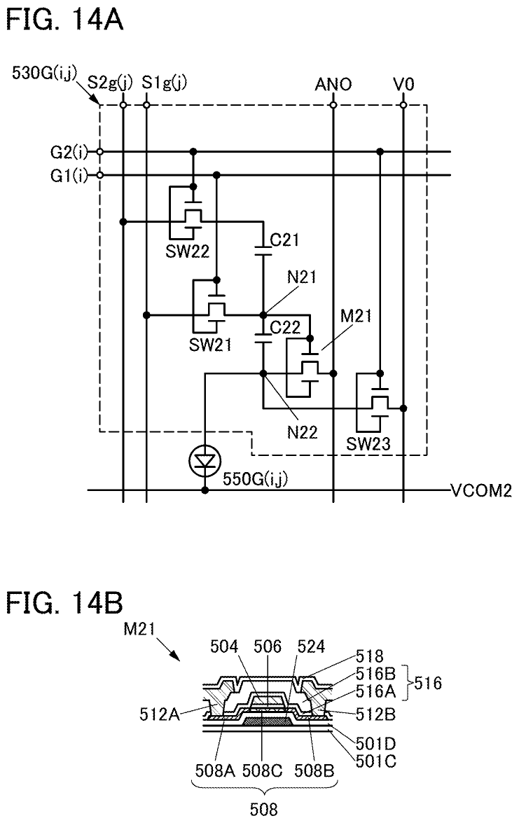

FIG. 14A illustrates a specific configuration of the pixel circuit 530G(i,j).

The pixel circuit 530G(i,j) includes a switch SW21, a switch SW22, a transistor M21, a capacitor C21, and a node N21 as illustrated in FIG. 14A. In addition, the pixel circuit 530G(i,j) includes a node N22, a capacitor C22, and a switch SW23.

The transistor M21 includes a gate electrode electrically connected to the node N21, a first electrode electrically connected to the light-emitting device 550G(i,j), and a second electrode electrically connected to a conductive film ANO.

The switch SW21 includes a first terminal electrically connected to the node N21 and a second terminal electrically connected to the conductive film S1g(j). The switch SW21 has a function of controlling its on/off state on the basis of the potential of the conductive film G1(i).

The switch SW22 includes a first terminal electrically connected to the conductive film S2g(j), and has a function of controlling the conduction state or the non-conduction state on the basis of a potential of the conductive film G2(i).

The capacitor C21 includes a conductive film electrically connected to the node N21 and a conductive film electrically connected to a second electrode of the switch SW22.

Thus, the image signal can be stored in the node N21. A potential of the node N21 can be changed using the switch SW22. Alternatively, the intensity of light emitted from the light-emitting device 550G(i,j) can be controlled with the potential of the node N21.

FIG. 14B illustrates an example of a specific structure of the transistor M21 described in FIG. 14A. As the transistor M21, a bottom-gate transistor, a top-gate transistor, or the like can be used as appropriate.

The transistor illustrated in FIG. 14B includes a semiconductor film 508, a conductive film 504, an insulating film 506, a conductive film 512A, and a conductive film 512B. The transistor is formed over an insulating film 501C, for example. The transistor also includes an insulating film 516 (an insulating film 516A and an insulating film 516B) and an insulating film 518.

The semiconductor film 508 includes a region 508A electrically connected to the conductive film 512A and a region 508B electrically connected to the conductive film 512B. The semiconductor film 508 includes a region 508C between the region 508A and the region 508B.

The conductive film 504 includes a region overlapping with the region 508C, and the conductive film 504 has a function of a gate electrode.

An insulating film 506 includes a region interposed between the semiconductor film 508 and the conductive film 504. The insulating film 506 has a function of a first gate insulating film.

The conductive film 512A has one of a function of a source electrode and a function of a drain electrode, and the conductive film 512B has the other of the function of the source electrode and the function of the drain electrode.

A conductive film 524 can be used for the transistor. The conductive film 524 includes a region where the semiconductor film 508 is interposed between the conductive film 524 and the conductive film 504. The conductive film 524 has a function of a second gate electrode. An insulating film 501D is interposed between the semiconductor film 508 and the conductive film 524, and has a function of a second gate insulating film.

The insulating film 516 functions as, for example, a protective film covering the semiconductor film 508. Specifically, a film including a silicon oxide film, a silicon oxynitride film, a silicon nitride oxide film, a silicon nitride film, an aluminum oxide film, a hafnium oxide film, an yttrium oxide film, a zirconium oxide film, a gallium oxide film, a tantalum oxide film, a magnesium oxide film, a lanthanum oxide film, a cerium oxide film, or a neodymium oxide film can be used as the insulating film 516.

For example, a material having a function of inhibiting diffusion of oxygen, hydrogen, water, an alkali metal, an alkaline earth metal, and the like is preferably used for the insulating film 518. Specifically, the insulating film 518 can be formed using silicon nitride, silicon oxynitride, aluminum nitride, or aluminum oxynitride, for example. In each of silicon oxynitride and aluminum oxynitride, the number of nitrogen atoms contained is preferably larger than the number of oxygen atoms contained.

Note that the semiconductor film used in the transistor of the driver circuit can be formed in the step of forming the semiconductor film used in the transistor of the pixel circuit. A semiconductor film having the same composition as the semiconductor film used in the transistor of the pixel circuit can be used in the driver circuit, for example.

For the semiconductor film 508, a semiconductor containing a Group 14 element can be used. Specifically, a semiconductor containing silicon can be used for the semiconductor film 508.

Hydrogenated amorphous silicon can be used for the semiconductor film 508. Alternatively, microcrystalline silicon or the like can be used for the semiconductor film 508. Thus, a light-emitting apparatus having less display unevenness than a light-emitting apparatus using polysilicon for the semiconductor film 508, for example, can be provided. It also facilitates the increase in size of the light-emitting apparatus.

Polysilicon can be used for the semiconductor film 508. In this case, the field-effect mobility of the transistor can be higher than that of a transistor using hydrogenated amorphous silicon for the semiconductor film 508, for example. The driving capability can be higher than that of a transistor using hydrogenated amorphous silicon for the semiconductor film 508, for example. The aperture ratio of the pixel can be higher than that in the case of using a transistor that uses hydrogenated amorphous silicon for the semiconductor film 508, for example.

The reliability of the transistor can be higher than that of a transistor using hydrogenated amorphous silicon for the semiconductor film 508, for example.

The temperature required for fabrication of the transistor can be lower than that required for a transistor using single crystal silicon, for example.

The semiconductor film used in the transistor of the driver circuit can be formed in the same step as the semiconductor film used in the transistor of the pixel circuit. The driver circuit can be formed over the same substrate where the pixel circuit is formed. The number of components included in an electronic device can be reduced.

Single crystal silicon can be used for the semiconductor film 508. In this case, a display panel with higher resolution than a light-emitting apparatus (or a display panel) using hydrogenated amorphous silicon for the semiconductor film 508, for example, can be provided. A light-emitting apparatus having less display unevenness than a light-emitting apparatus using polysilicon for the semiconductor film 508, for example, can be provided. Smart glasses or a head-mounted display can be provided, for example.

A metal oxide can be used for the semiconductor film 508. In this case, the pixel circuit can hold an image signal for a longer time than a pixel circuit utilizing a transistor using amorphous silicon for a semiconductor film. Specifically, a selection signal can be supplied at a frequency of lower than 30 Hz, preferably lower than 1 Hz, further preferably less than once per minute with the suppressed occurrence of flickers. Thus, cumulative fatigue stored in a user of an electronic device user can be reduced. Moreover, power consumption for driving can be reduced.

An oxide semiconductor can be used for the oxide semiconductor film 508. Specifically, an oxide semiconductor containing indium, an oxide semiconductor containing indium, gallium, and zinc, or an oxide semiconductor containing indium, gallium, zinc, and tin can be used for the semiconductor film 508.

The use of an oxide semiconductor for the semiconductor film achieves a transistor having lower leakage current in the off state than a transistor using amorphous silicon for the semiconductor film. Specifically, a transistor using an oxide semiconductor for a semiconductor film is preferably used as a switch or the like. Note that a circuit in which a transistor using an oxide semiconductor for the semiconductor film is used as a switch is capable of retaining the potential of a floating node for a longer time than a circuit in which a transistor using amorphous silicon for the semiconductor film is used as a switch.

Although the light-emitting apparatus in FIG. 15A has a structure in which light is extracted from the second substrate 770 side (a top-emission structure), the light-emitting apparatus may have a structure in which light is extracted from the first substrate 510 side (a bottom-emission structure) as shown in FIG. 15B. For a bottom-emission light-emitting apparatus, a lower electrode of a pair of electrodes is formed so as to function as a transflective electrode and the upper electrode of the pair of electrodes is formed so as to function as a reflective electrode.

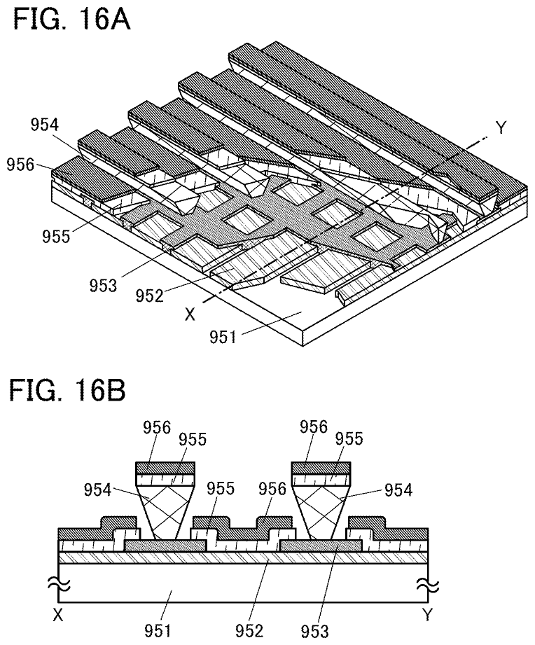

Although FIGS. 15A and 15B illustrate active-matrix light-emitting apparatuses, the structure of the light-emitting device described in Embodiment 1 may be applied to a passive-matrix light-emitting apparatus illustrated in FIGS. 16A and 16B.

Note that FIG. 16A is a perspective view of a passive matrix light-emitting apparatus, and FIG. 16B is a cross-sectional view taken along the line X-Y in FIG. 16A. In FIG. 16A and FIG. 16B, an electrode 952 and an electrode 956 are provided over a substrate 951, and an EL layer 955 is provided between the electrode 952 and the electrode 956. An end portion of the electrode 952 is covered with an insulating layer 953. A partition layer 954 is provided over the insulating layer 953. Sidewalls of the partition layer 954 are aslope such that the distance between one sidewall and the other sidewall is gradually narrowed toward the surface of the substrate. That is, a cross section in the short side direction of the partition layer 954 is a trapezoidal shape, and the lower side (the side facing the same direction as the plane direction of the insulating layer 953 and touching the insulating layer 953) is shorter than the upper side (the side facing the same direction as the plane direction of the insulating layer 953, and not touching the insulating layer 953). By providing the partition layer 954 in this manner, defects of the light-emitting device due to static charge or the like can be prevented.

Note that the structure described in this embodiment can be used in an appropriate combination with any of the structures described in the other embodiments.

### Embodiment 5

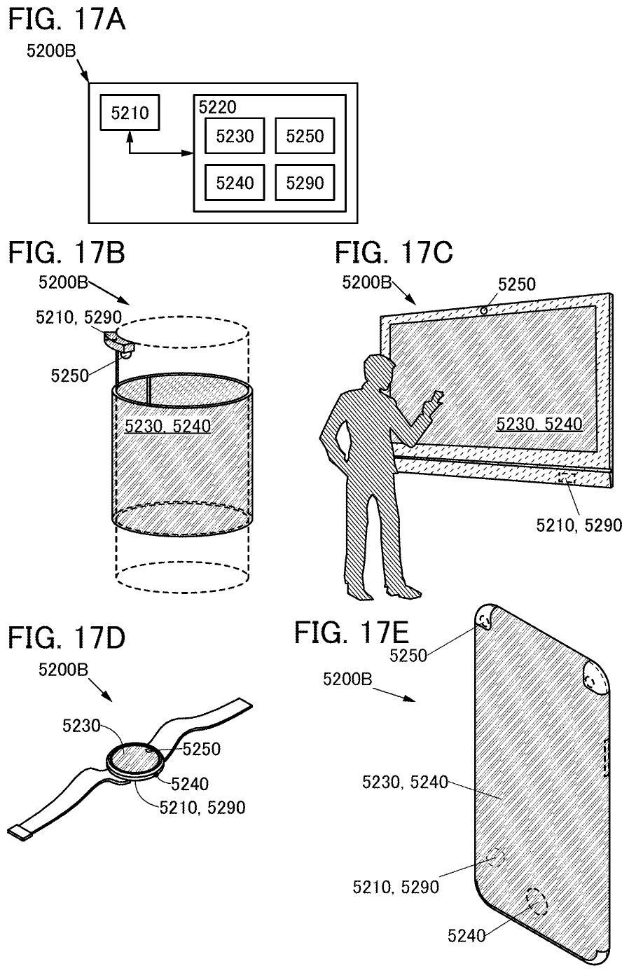

In this embodiment, electronic devices of one embodiment of the present invention will be described with reference to FIG. 17A to FIG. 19B.

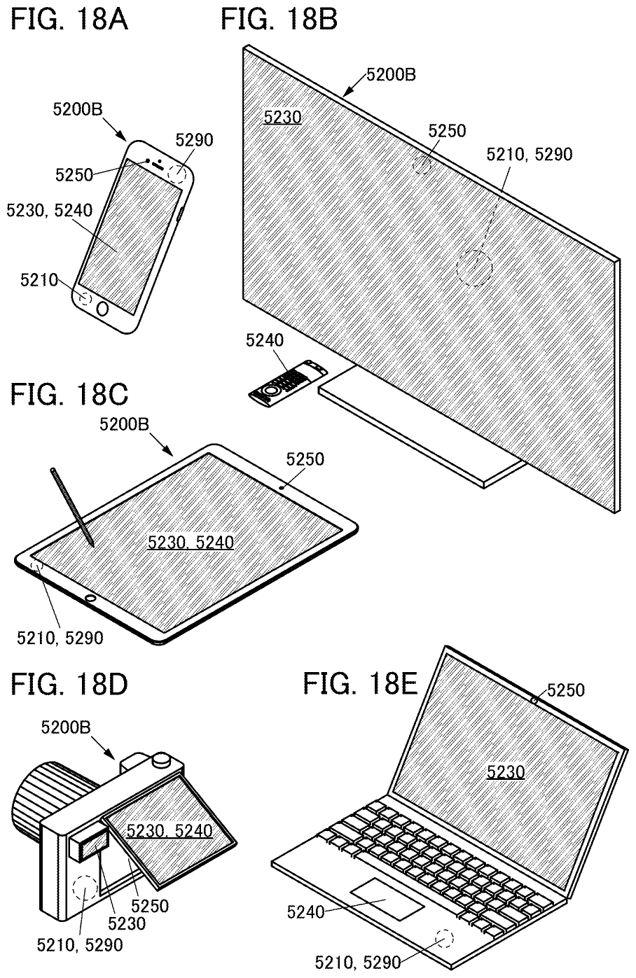

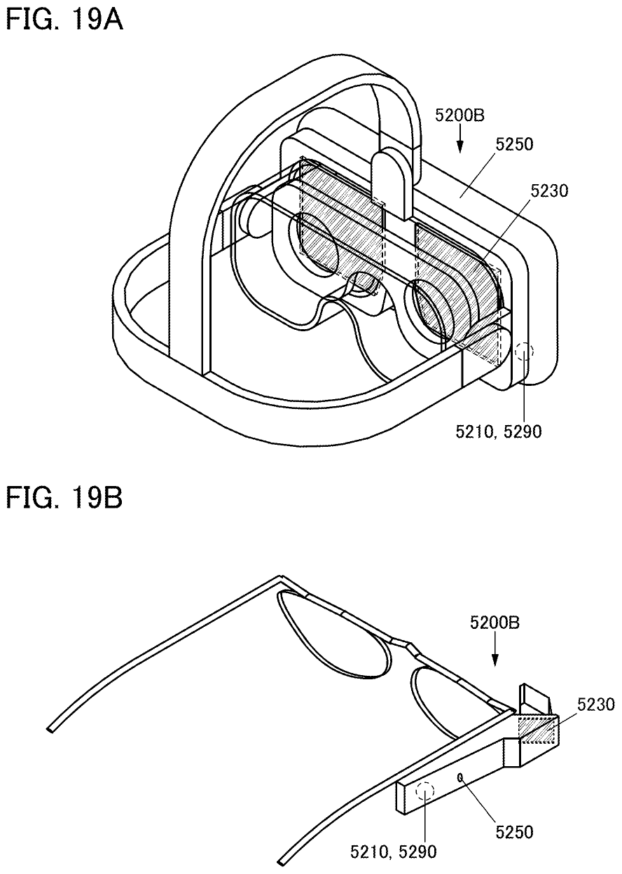

FIG. 17A to FIG. 19B are diagrams illustrating structures of electronic devices of one embodiment of the present invention. FIG. 17A is a block diagram of the electronic device and FIG. 17B to FIG. 17E are perspective views illustrating structures of the electronic devices. FIG. 18A to FIG. 18E are perspective views illustrating structures of electronic devices. FIG. 19A and FIG. 19B are perspective views illustrating structures of an electronic device.

An electronic device 5200B described in this embodiment includes an arithmetic device 5210 and an input/output device 5220 (see FIG. 17A).

The arithmetic device 5210 has a function of being supplied with operation information and a function of supplying image information on the basis of the operation information.

The input/output device 5220 includes a display portion 5230, an input portion 5240, a sensing portion 5250, and a communication portion 5290 and has a function of supplying operation information and a function of being supplied with image information. The input/output device 5220 also has a function of supplying sensing information, a function of supplying communication information, and a function of being supplied with communication information.

The input portion 5240 has a function of supplying operation information. For example, the input portion 5240 supplies operation information on the basis of operation by a user of the electronic device 5200B.

Specifically, a keyboard, a hardware button, a pointing device, a touch sensor, an illuminance sensor, an imaging device, an audio input device, an eye-gaze input device, an attitude detection device, or the like can be used as the input portion 5240.

The display portion 5230 includes a display panel and has a function of displaying image information. For example, the display panel described in Embodiment 2 can be used for the display portion 5230.

The sensing portion 5250 has a function of supplying sensing information. For example, the sensing portion 5250 has a function of sensing a surrounding environment where the electronic device is used and supplying sensing information.

Specifically, an illuminance sensor, an imaging device, an attitude detection device, a pressure sensor, a human motion sensor, or the like can be used as the sensing portion 5250.

The communication portion 5290 has a function of being supplied with communication information and a function of supplying communication information. For example, the communication portion 5290 has a function of being connected to another electronic device or a communication network through wireless communication or wired communication. Specifically, the communication portion 5290 has a function of wireless local area network communication, telephone communication, near field communication, or the like.

FIG. 17B illustrates an electronic device having an outer shape along a cylindrical column or the like. An example of such an electronic device is digital signage. The display panel of one embodiment of the present invention can be used for a display portion 5230. In addition, the electronic device may have a function of changing its display method in accordance with the illuminance of a usage environment. Furthermore, the electronic device has a function of changing displayed content in response to sensed existence of a person. This allows the electronic device to be provided on a column of a building, for example. The electronic device can display advertising, guidance, or the like. The electronic device can be used for digital signage or the like.

FIG. 17C illustrates an electronic device having a function of generating image data on the basis of the path of a pointer used by the user. Examples of such an electronic device include an electronic blackboard, an electronic bulletin board, and digital signage. Specifically, the display panel with a diagonal size of 20 inches or longer, preferably 40 inches or longer, further preferably 55 inches or longer can be used. Alternatively, a plurality of display panels can be arranged and used as one display region. Alternatively, a plurality of display panels can be arranged and used as a multiscreen.

FIG. 17D illustrates an electronic device that is capable of receiving data from another device and displaying the data on the display portion 5230. An example of such an electronic device is a wearable electronic device. Specifically, the electronic device can display several options, and the user can choose some from the options and send a reply to the data transmitter. For example, the electronic device has a function of changing its display method in accordance with the illuminance of a usage environment. Thus, for example, the power consumption of a wearable electronic device can be reduced. Alternatively, for example, a wearable electronic device can display an image so as to be suitably used even in an environment under strong external light, e.g., outdoors in fine weather.

FIG. 17E illustrates an electronic device including the display portion 5230 having a surface gently curved along a side surface of a housing. An example of such an electronic device is a mobile phone. The display portion 5230 includes a display panel, and the display panel has a function of performing display on the front surface, the side surfaces, the top surface, and the rear surface, for example. Thus, for example, a mobile phone can display information not only on its front surface but also on its side surfaces, its top surface, and its rear surface.

FIG. 18A illustrates an electronic device that is capable of receiving data via the Internet and displaying the data on the display portion 5230. An example of such an electronic device is a smartphone. A created message can be checked on the display portion 5230, for example. The created message can be sent to another device. For example, the electronic device has a function of changing its display method in accordance with the illuminance of a usage environment. Thus, the power consumption of a smartphone can be reduced. Alternatively, for example, a smartphone can display an image so as to be suitably used even in an environment under strong external light, e.g., outdoors in fine weather.

FIG. 18B illustrates an electronic device that can use a remote controller as an input portion 5240. An example of such an electronic device is a television system. For example, the electronic device can receive information from a broadcast station or via the Internet and display the information on the display portion 5230. An image of a user can be captured using the sensing portion 5250. The image of the user can be transmitted. The electronic device can acquire a viewing history of the user and provide it to a cloud service. The electronic device can acquire recommendation information from a cloud service and display the information on the display portion 5230. A program or a moving image can be displayed on the basis of the recommendation information. For example, the electronic device has a function of changing its display method in accordance with the illuminance of a usage environment. Accordingly, for example, a television system can display an image to be suitably used even when irradiated with strong external light that enters a room in fine weather.

FIG. 18C illustrates an electronic device that is capable of receiving educational materials via the Internet and displaying them on the display portion 5230. An example of such an electronic device is a tablet computer. An assignment can be input with the input portion 5240 and sent via the Internet. A corrected assignment or the evaluation of the assignment can be obtained from a cloud service and displayed on the display portion 5230. Suitable educational materials can be selected on the basis of the evaluation and displayed.

For example, the display portion 5230 can perform display using an image signal received from another electronic device. When the electronic device is placed on a stand or the like, the display portion 5230 can be used as a sub-display. Thus, for example, a tablet computer can display an image to be suitably used even in an environment under strong external light, e.g., outdoors in fine weather.

FIG. 18D illustrates an electronic device including a plurality of display portions 5230. An example of such an electronic device is a digital camera. For example, the display portion 5230 can display an image that the sensing portion 5250 is capturing. A captured image can be displayed on the sensing portion. A captured image can be decorated using the input portion 5240. A message can be attached to a captured image. A captured image can be transmitted via the Internet. The electronic device has a function of changing its shooting conditions in accordance with the illuminance of a usage environment. Accordingly, for example, a digital camera can display a subject in such a manner that an image is favorably viewed even in an environment under strong external light, e.g., outdoors in fine weather.

FIG. 18E illustrates an electronic device in which the electronic device of this embodiment is used as a master to control another electronic device used as a slave. An example of such an electronic device is a portable personal computer. As another example, part of image data can be displayed on the display portion 5230 and another part of the image data can be displayed on a display portion of another electronic device. An image signal can be supplied. With the communication portion 5290, information to be written can be obtained from an input portion of another electronic device. Thus, a large display region can be utilized by using a portable personal computer, for example.

FIG. 19A illustrates an electronic device including the sensing portion 5250 that senses an acceleration or a direction. An example of such an electronic device is a goggles-type electronic device. The sensing portion 5250 can supply information on the position of the user or the direction in which the user faces. The electronic device can generate image information for the right eye and image information for the left eye in accordance with the position of the user or the direction in which the user faces. The display portion 5230 includes a display region for the right eye and a display region for the left eye. Thus, a virtual reality image that gives the user a sense of immersion can be displayed on a goggles-type electronic device, for example.

FIG. 19B illustrates an electronic device including an imaging device and the sensing portion 5250 that senses an acceleration or a direction. An example of such an electronic device is a glasses-type electronic device. The sensing portion 5250 can supply information on the position of the user or the direction in which the user faces. The electronic device can generate image information in accordance with the position of the user or the direction in which the user faces. Accordingly, the information can be shown together with a real-world scene, for example. An augmented reality image can be displayed on a glasses-type data electronic device.

Note that this embodiment can be combined with any of the other embodiments described in this specification as appropriate.

### Embodiment 6

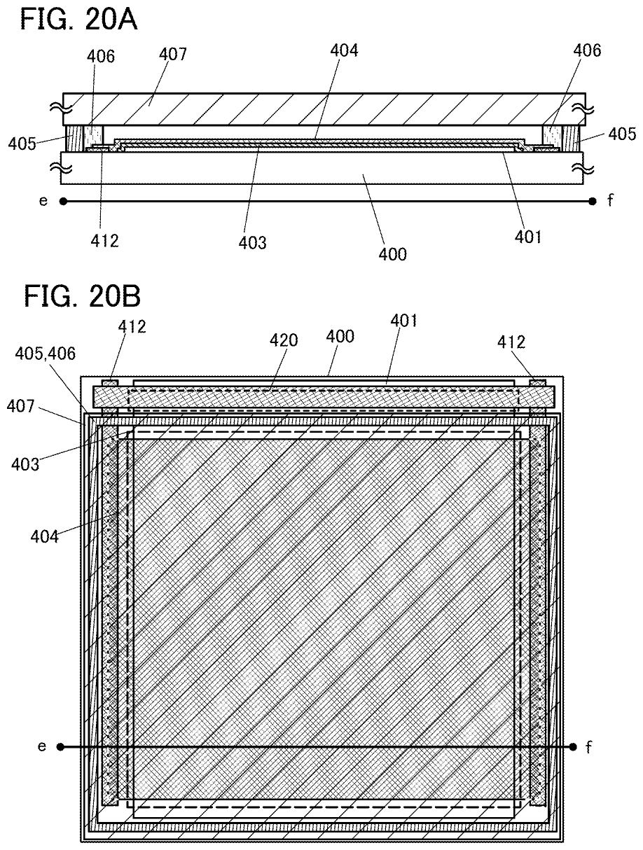

In this embodiment, a structure in which the light-emitting device described in Embodiment 2 is used for a lighting device will be described with reference to FIG. 20A and FIG. 20B. FIG. 20A shows a cross section taken along the line e-f in a top view of the lighting device in FIG. 20B.

In the lighting device in this embodiment, a first electrode 401 is formed over a substrate 400 which is a support and has a light-transmitting property. The first electrode 401 corresponds to the first electrode 101 in Embodiment 2. In the case where light emission is extracted from the first electrode 401 side, the first electrode 401 is formed with a material having a light-transmitting property.

A pad 412 for supplying a voltage to a second electrode 404 is formed over the substrate 400.

An EL layer 403 is formed over the first electrode 401. The structure of the EL layer 403 corresponds to, for example, the structure of the EL layer 103 in Embodiment 2 or the structure in which the EL layers 103a, 103b, and 103c and the charge-generation layers 106 (106a and 106b) are combined. Note that for these structures, the corresponding description can be referred to.

The second electrode 404 is formed to cover the EL layer 403. The second electrode 404 corresponds to the second electrode 102 in Embodiment 2. In the case where light-emission is extracted from the first electrode 401 side, the second electrode 404 is formed with a material having high reflectivity. The second electrode 404 is supplied with a voltage when connected to the pad 412.

As described above, the lighting device described in this embodiment includes a light-emitting device including the first electrode 401, the EL layer 403, and the second electrode 404. Since the light-emitting device is a light-emitting device with high emission efficiency, the lighting device in this embodiment can be a lighting device with low power consumption.

The substrate 400 over which the light-emitting device having the above structure is formed is fixed to a sealing substrate 407 with sealants 405 and 406 and sealing is performed, whereby the lighting device is completed. It is possible to use only either the sealant 405 or 406. In addition, the inner sealant 406 (not shown in FIG. 20B) can be mixed with a desiccant, which enables moisture to be adsorbed, resulting in improved reliability.

When parts of the pad 412 and the first electrode 401 are provided to extend to the outside of the sealants 405 and 406, those can serve as external input terminals. An IC chip 420 mounted with a converter or the like may be provided over the external input terminals.

### Embodiment 7

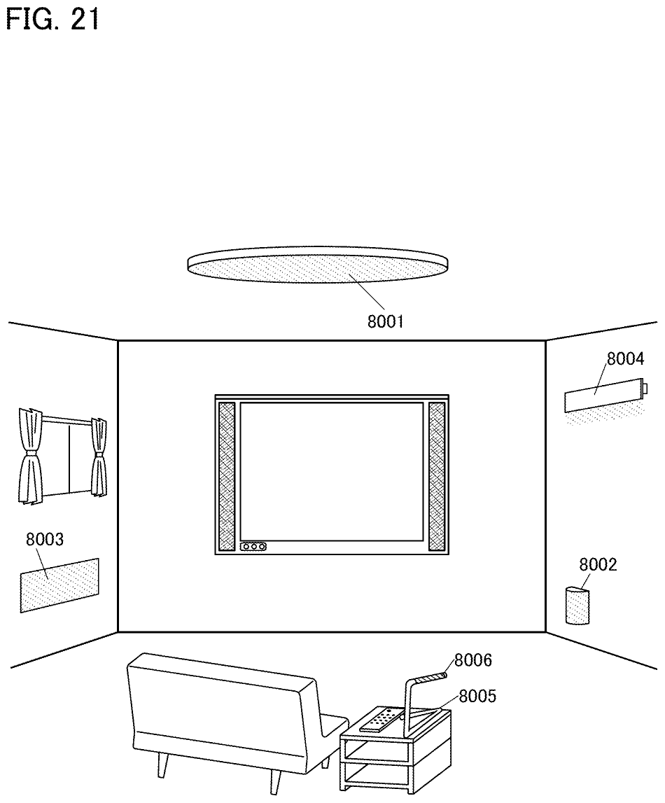

In this embodiment, an application example of a lighting device fabricated using the light-emitting apparatus of one embodiment of the present invention or the light-emitting device which is part of the light-emitting apparatus will be described with reference to FIG. 21.

A ceiling light 8001 can be used as an indoor lighting device. As the ceiling light 8001, a direct-mount light or an embedded light is given. Such a lighting device is fabricated using the light-emitting apparatus and a housing or a cover in combination. Besides, application to a cord pendant light (light that is suspended from a ceiling by a cord) is also possible.

A foot light 8002 lights a floor so that safety on the floor can be improved. For example, it can be effectively used in a bedroom, on a staircase, on a passage, or the like. In such a case, the size and shape of the foot light can be changed depending on the area or structure of a room. The foot light can also be a stationary lighting device fabricated using the light-emitting apparatus and a support base in combination.

A sheet-like lighting 8003 is a thin sheet-like lighting device. The sheet-like lighting, which is attached to a wall when used, is space-saving and thus can be used for a wide variety of uses. The area of the sheet-like lighting can be easily increased. The sheet-like lighting can also be used on a wall and a housing that have a curved surface.

In addition, a lighting device 8004 in which the direction of light from a light source is controlled to be only a desired direction can be used.

A desk lamp 8005 includes a light source 8006. As the light source 8006, the light-emitting apparatus of one embodiment of the present invention or the light-emitting device, which is part of the light-emitting apparatus, can be used.

Besides the above examples, when the light-emitting apparatus of one embodiment of the present invention or the light-emitting device which is a part thereof is used as part of furniture in a room, a lighting device that functions as the furniture can be obtained.

As described above, a variety of lighting devices that include the light-emitting apparatus can be obtained. Note that these lighting devices are also embodiments of the present invention.

The structures described in this embodiment can be used in an appropriate combination with any of the structures described in the other embodiments.

### Example 1

### Synthesis Example 1

In this example, will be described a synthesis method of 2,8-bis(3,6-di-tert-butyl-9H-carbazol-9-yl)-11-methyl-Benzo[1″,2″:4,5;5″,4″:4′,5′]difuro[2,3-b:2′,3′-b′]dipyridine (abbreviation: 2,8tBuCz2Bdfpy), an organic compound of one embodiment of the present invention, which is represented by Structural Formula (100) in Embodiment 1. A structure of 2,8tBuCz2Bdfpy is shown below.

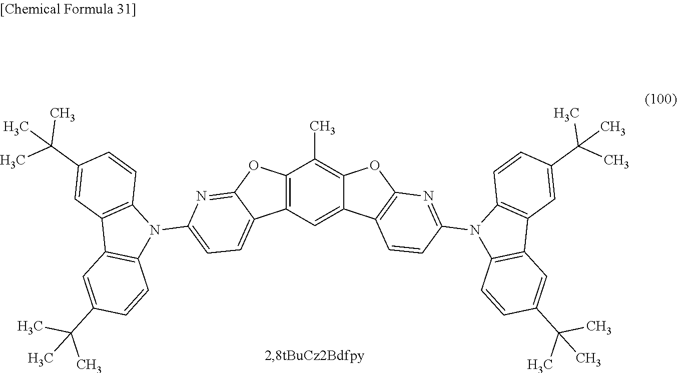 [SMILES: Cc1c2oc3nc(-n4c5ccc(C(C)(C)C)cc5c5cc(C(C)(C)C)ccc54)ccc3c2cc2c1oc1nc(-n3c4ccc(C(C)(C)C)cc4c4cc(C(C)(C)C)ccc43)ccc12]

### Step 1: Synthesis of 2,2′-[(2-methyl-1,3-phenylene)bis(oxy)]bis(6-dichloropyridine)

Into a 200 mL recovery flask, 2.4 g (19 mmol) of 2-methylresorcinol, 5.0 g (38 mmol) of 2-chloro-6-fluoropyridine, 12 g (38 mmol) of cesium carbonate, and 120 mL of N,N-dimethylformamide (DMF) were put. This mixture was stirred at 90° C. under a nitrogen stream for 4 hours. After a predetermined time elapsed, the reaction mixture was poured into 200 mL of water, whereby a solid was precipitated. The precipitated solid was subjected to suction filtration to give 3.5 g of a white solid in a yield of 52%. The obtained white solid was identified as 2,2′-[(2-methyl-1,3-phenylene)bis(oxy)]bis(6-dichloropyridine) by nuclear magnetic resonance (NMR) spectroscopy. The synthesis scheme in Step 1 is shown in Formula (a-1) below.

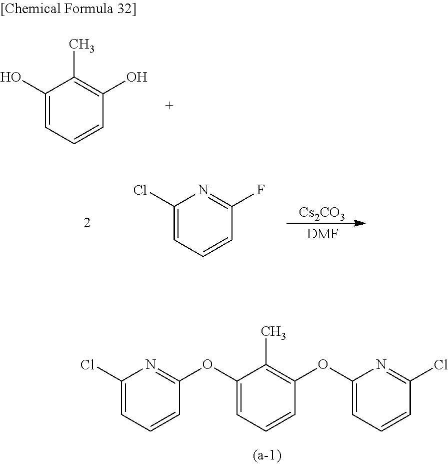

### Step 2: Synthesis of 2,8-dichloro-11-methyl-Benzo[1″,2″:4,5;5″,4″:4′,5′]difuro[2,3-b:2′,3′-b′]dipyridine

Into a 200 mL three-neck flask, 3.5 g (10 mmol) of 2,2′-[(2-methyl-1,3-phenylene)bis(oxy)]bis(6-dichloropyridine) synthesized in Step 1, 50 g of pivalic acid, 1.0 g (3.0 mmol) of palladium trifluoroacetate (Pd(TFA)₂), and 6.6 g (40 mmol) of silver acetate were put, and the mixture was stirred at 150° C. for 33 hours in air. After a predetermined time elapsed, the reaction mixture was added with ethyl acetate and suction-filtrated to give a solid.

Toluene was added to the resulting solid, and the resulting solid was heated, dissolved and filtered through Celite. The obtained filtrate was concentrated to give a solid. A mixed solvent of ethyl acetate and hexane was added to the solid, the mixture was subjected to suction filtration, whereby 0.26 g of a yellow solid was obtained in a yield of 8%. The obtained yellow solid was identified as 2,8-dichloro-11-methyl-Benzo[1″,2″:4,5;5″,4″:4′,5′]difuro[2,3-b:2′,3′-b′]dipyridine by nuclear magnetic resonance (NMR) spectroscopy. The synthesis scheme in Step 2 is shown in Formula (a-2) below.

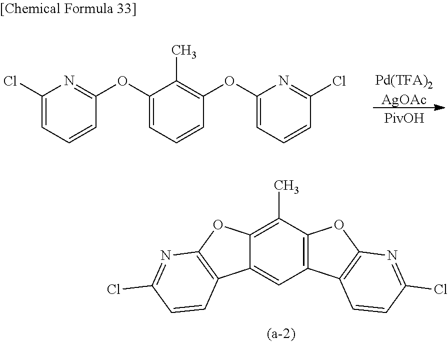

### Step 3: Synthesis of 2,8tBuCz2Bdfpy

Into a 200 mL three-neck flask, 0.26 g (0.76 mmol) of 2,8-dichloro-11-methyl-Benzo[1″,2″:4,5;5″,4″:4′,5′]difuro[2,3-b:2′,3′-b]dipyridine, 0.47 g (1.7 mmol) of 3,6-di-tert-butlycarbazole, 0.32 g (3.3 mmol) of sodium tert-butoxide, and 30 mL of xylene were put, and the air in the flask was replaced with nitrogen. After that, the mixture was degassed by being stirred while the pressure in the flask was reduced. After the degassing, 34 mg of allylpalladium(II)chloride dimer ([Pd(allyl)Cl]₂) and 10 mg of di-tert-butyl(1-methyl-2,2-diphenylcyclopropyl)phosphine (cBRIDP) were added to the mixture and was stirred while being heated at 130° C. for 7 hours under a nitrogen stream.

After a predetermined time elapsed, the reaction mixture was filtered through Celite. The obtained filtrate was concentrated to give a solid. Ethanol was added to the resulting solid, which was then suction-filtered to give a solid. Toluene was added to the obtained solid and the mixture was dissolved by heating. Then, the mixture was filtered through a filter aid in which Celite, alumina, and Celite were stacked in this order. The obtained filtrate was concentrated to give a solid. The obtained solid was recrystallized with a mixed solvent of toluene and ethanol, whereby 0.33 g of a white solid was obtained in a yield of 52%. By a train sublimation method, 0.32 g of the obtained solid was sublimated and purified. The purification by sublimation was performed by heating at 365° C. for 19 hours under a pressure of 2.4 Pa with an argon flow rate of 10.5 mL/min for 19 hours. After the sublimation purification, 0.17 g of a white solid was obtained at a collection rate of 52%. The synthesis scheme in Step 3 is shown in Formula (a-3) below.

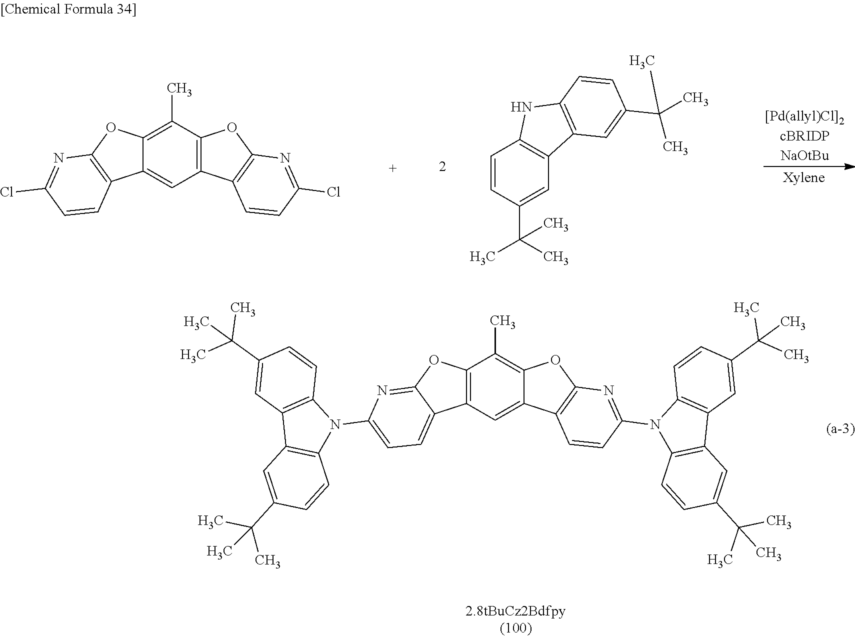 [SMILES: Cc1c2oc3nc(Cl)ccc3c2cc2c1oc1nc(Cl)ccc12 ; CC(C)(C)c1ccc2[nH]c3ccc(C(C)(C)C)cc3c2c1 ; Cc1c2oc3nc(-n4c5ccc(C(C)(C)C)cc5c5cc(C(C)(C)C)ccc54)ccc3c2cc2c1oc1nc(-n3c4ccc(C(C)(C)C)cc4c4cc(C(C)(C)C)ccc43)ccc12]

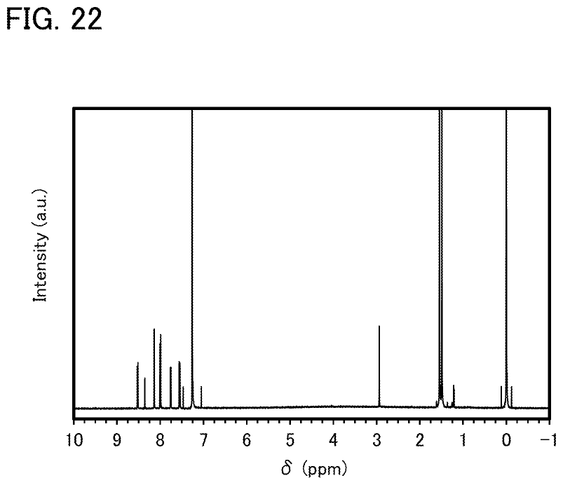

Protons (¹H) of the white solid obtained above were measured by nuclear magnetic resonance (NMR) spectroscopy. The obtained values are shown below. FIG. 22 shows ¹H-NMR charts. The results revealed that 2,8tBuCz2Bdfpy represented by Structural Formula (100) above was obtained in this example.

¹H-NMR. δ (CDCl₃): 1.49 (s, 36H), 2.94 (s, 3H), 7.55 (d, 4H), 7.76 (d, 2H), 8.00 (d, 4H), 8.14 (s, 4H), 8.36 (s, 1H), 8.52 (d, 2H).

Next, the ultraviolet-visible absorption spectra (hereinafter, simply referred to as “absorption spectra”) and emission spectra of 2,8tBuCz2Bdfpy in a toluene solution and a solid thin film were measured.

The absorption spectrum of 2,8tBuCz2Bdfpy in the toluene solution was measured with an ultraviolet-visible spectrophotometer (V550, produced by JASCO Corporation). The absorption spectrum in the toluene solution was obtained by subtracting the measured absorption spectrum of only toluene put in a quartz cell from the measured absorption spectrum of the toluene solution with 2,8tBuCz2Bdfpy put in a quartz cell. The emission spectrum in the toluene solution was measured with a fluorescence spectrophotometer (FP-8600, produced by JASCO Corporation).

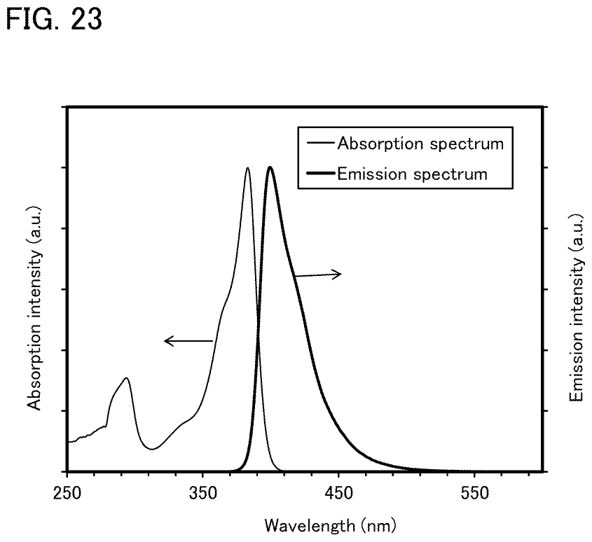

FIG. 23 shows the obtained measurement results of the absorption spectrum and the emission spectrum of 2,8tBuCz2Bdfpy in the toluene solution. The horizontal axis represents wavelengths and the vertical axes represent absorption intensity and emission intensity. As results shown in FIG. 23, the toluene solution with 2,8tBuCz2Bdfpy exhibited absorption peaks at around 383 nm, 365 nm, 336 nm, and 295 nm and an emission peak at around 400 nm (excitation wavelength: 350 nm).

For the measurement of the absorption spectrum of 2,8tBuCz2Bdfpy in the solid thin film state, a spectrophotometer (U4100 Spectrophotometer, manufactured by Hitachi High-Technologies Corporation) was used. The solid thin film was formed over a quartz substrate by a vacuum evaporation method. The emission spectrum of the solid thin film was measured with a fluorescence spectrophotometer (FP-8600, produced by JASCO Corporation).

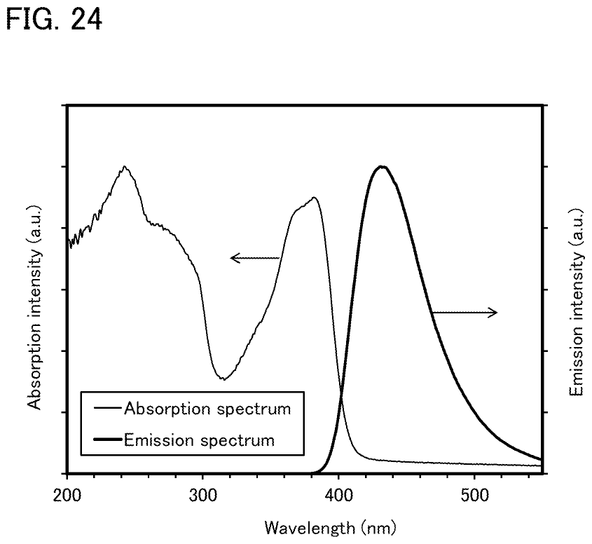

FIG. 24 shows the absorption spectrum and the emission spectrum of the obtained solid thin film of 2,8tBuCz2Bdfpy. The horizontal axis represents wavelengths and the vertical axes represent absorption intensity and emission intensity. As results shown in FIG. 24, the solid thin film of 2,8tBuCz2Bdfpy exhibited absorption peaks at around 381 nm, 370 nm, 340 nm, 275 nm, and 242 nm and an emission peak at around 431 nm (excitation wavelength: 360 nm).

These results indicate that 2,8tBuCz2Bdfpy emits blue light and can also be used as a host for a light-emitting substance or a host for a substance that emits fluorescence in the visible region.

Moreover, the quantum yield in the toluene solution with 2,8tBuCz2Bdfpy was measured. For the measurement of the quantum yield, an absolute PL quantum yield measurement system (Quantaurus-QY, manufactured by Hamamatsu Photonics K.K.) was used.

Furthermore, the measured quantum yield in the toluene solution was as very high as 92%, which indicates that 2,8tBuCz2Bdfpy is suitable as a light-emitting material.

### Example 2

### Synthesis Example 2

In this example, will be described bis {N-9-(3,5-di-tert-butylphenyl)-9H-carbazol-2-yl]-N-phenyl}-11-methyl-benzo[1″,2″:4,5;5″,4″: 4′,5′]difuro[2,3-b:2′,3′-b′]dipyridine-2,8-diamine (abbreviation: 2,8mmtBuPCA2Bdfpy), an organic compound of one embodiment of the present invention, which is represented by Structural Formula (101) in Embodiment 1. A structure of 2,8mmtBuPCA2Bdfpy is shown below.

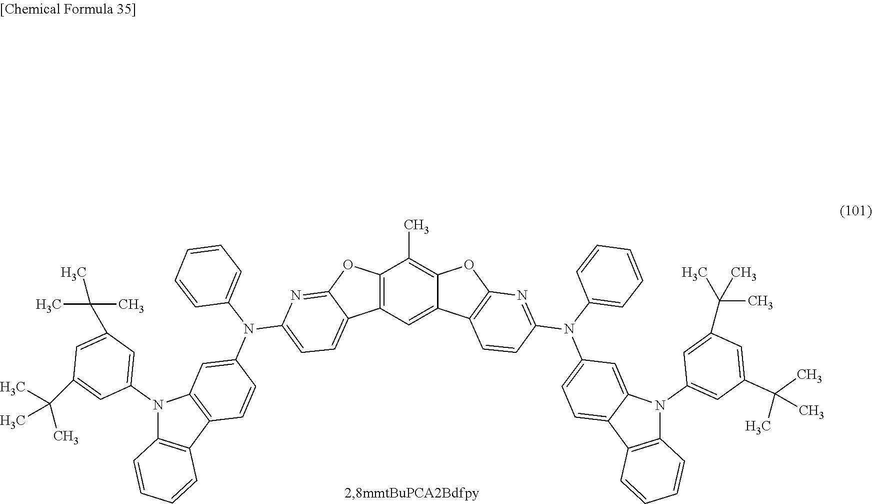 [SMILES: Cc1c2oc3nc(N(c4ccccc4)c4ccc5c6ccccc6n(-c6cc(C(C)(C)C)cc(C(C)(C)C)c6)c5c4)ccc3c2cc2c1oc1nc(N(c3ccccc3)c3ccc4c5ccccc5n(-c5cc(C(C)(C)C)cc(C(C)(C)C)c5)c4c3)ccc12]

### Step 1: Synthesis of 2,8mmtBuPCA2Bdfpy

Into a 200 mL three-neck flask, 0.5 g (1.5 mmol) of 2,8-dichloro-11-methyl-benzo[1″,2″:4,5;5″,4″:4′,5′]difuro[2,3-b:2′,3′-b]dipyridine synthesized in Step 2 of Synthesis example 1 shown in Example 1, 1.6 g (3.5 mmol) of N-[9-(3,5-di-tert-butylphenyl)-9H-carbazol-2-yl]-N-phenylamine, 0.84 g (8.8 mmol) of sodium tert-butoxide, 52 mg (0.15 mmol) of di(1-adamantyl)-n-butylphosphine (cataCXium A), and 100 mL of xylene were put, and the air in the flask was replaced with nitrogen. After that, the mixture was degassed by being stirred while the pressure in the flask was reduced. After the degassing, 26 mg (0.030 mmol) of tris(dibenzylideneacetone)dipalladium(0) (Pd₂(dba)₃) was added, and the mixture was stirred while being heated at 140° C. for 15 hours under a nitrogen stream.

After a predetermined time elapsed, the reaction mixture was filtered through a filter aid in which Celite, alumina, and Celite were stacked in this order. The obtained filtrate was concentrated to give a solid. The obtained solid was purified by silica column chromatography. Toluene was used as the developing solvent. The obtained fraction was concentrated to give a solid. The obtained solid was recrystallized with a mixed solvent of toluene and ethanol, whereby 0.92 g of a yellow solid was obtained in a yield of 54%. By a train sublimation method, 0.90 g of the obtained solid was sublimated and purified. The purification by sublimation was performed by heating at 380° C. under a pressure of 1.1×10⁻³ Pa for 17 hours. After the sublimation purification, 0.62 g of a yellow solid was obtained at a collection rate of 69%. The synthesis scheme in Step 1 is shown in Formula (b-1) below.

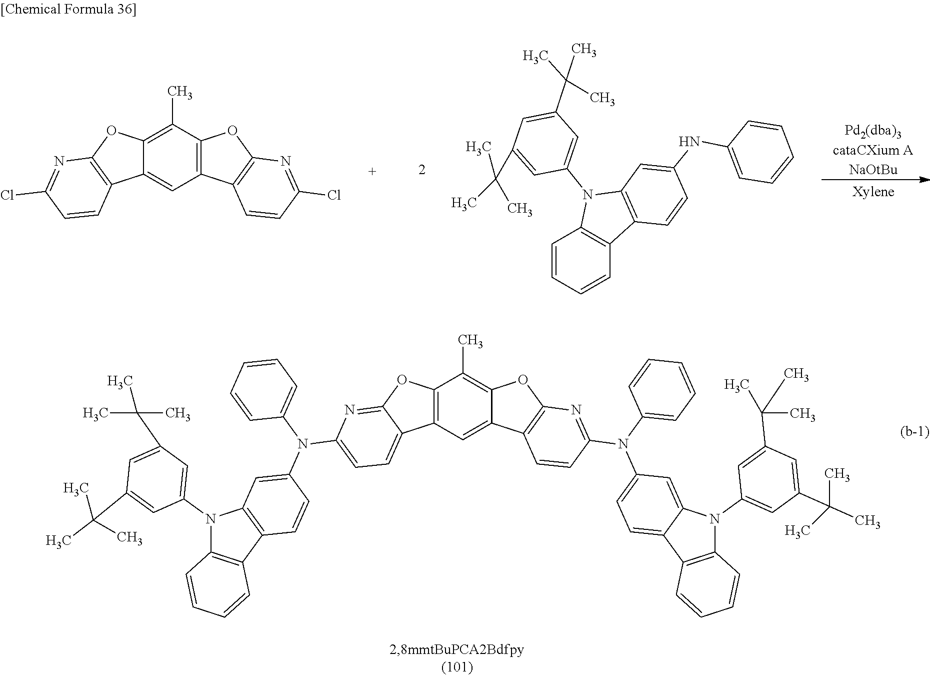 [SMILES: Cc1c2oc3nc(Cl)ccc3c2cc2c1oc1nc(Cl)ccc12 ; CC(C)(C)c1cc(-n2c3ccccc3c3ccc(Nc4ccccc4)cc32)cc(C(C)(C)C)c1 ; Cc1c2oc3nc(N(c4ccccc4)c4ccc5c6ccccc6n(-c6cc(C(C)(C)C)cc(C(C)(C)C)c6)c5c4)ccc3c2cc2c1oc1nc(N(c3ccccc3)c3ccc4c5ccccc5n(-c5cc(C(C)(C)C)cc(C(C)(C)C)c5)c4c3)ccc12]

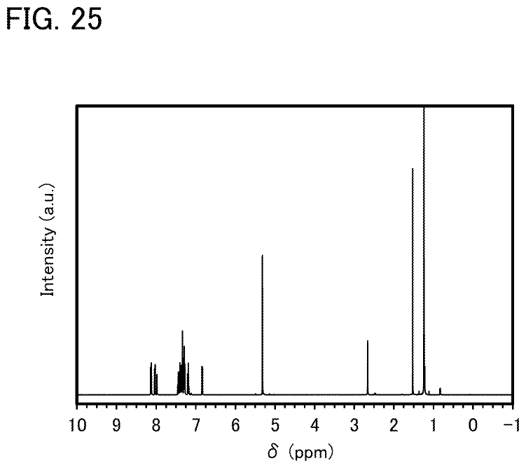

Protons (¹H) of the yellow solid obtained above were measured by nuclear magnetic resonance (NMR) spectroscopy. The obtained values are shown below. FIG. 25 shows the ¹H-NMR chart. The results revealed that 2,8mmtBuPCA2Bdfpy represented by Structural Formula (101) above was obtained in this example.

¹H-NMR. δ (CD₂Cl₂): 1.24 (s, 36H), 2.66 (s, 3H), 6.84 (d, 2H), 7.16-7.21 (m, 4H), 7.28-7.46 (m, 22H), 7.98 (s, 1H), 8.03 (d, 2H), 8.13 (d, 4H).

Next, absorption spectra and emission spectra of 2,8mmtBuPCA2Bdfpy in a toluene solution and a solid thin film were measured.

The absorption spectrum of 2,8mmtBuPCA2Bdfpy in the toluene solution was measured with an ultraviolet-visible spectrophotometer (V550, produced by JASCO Corporation). The absorption spectrum in the toluene solution was obtained by subtracting the measured absorption spectrum of only toluene put in a quartz cell from the measured absorption spectrum of the toluene solution with 2,8mmtBuPCA2Bdfpy put in a quartz cell. The emission spectrum in the toluene solution was measured with a fluorescence spectrophotometer (FP-8600, produced by JASCO Corporation).

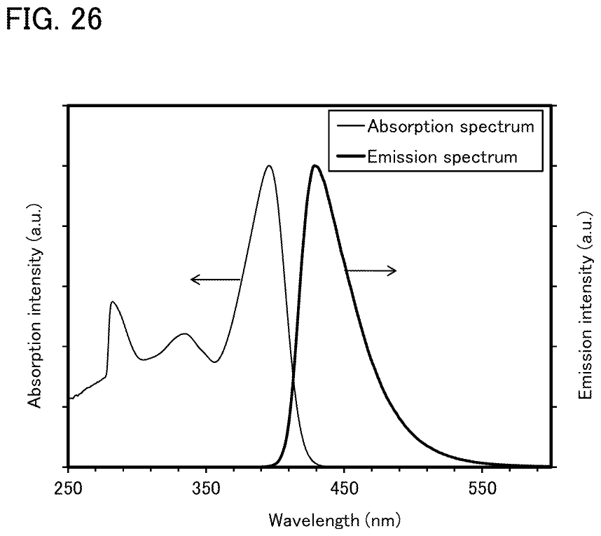

FIG. 26 shows the obtained measurement results of the absorption spectrum and the emission spectrum of 2,8mmtBuPCA2Bdfpy in the toluene solution. The horizontal axis represents wavelengths and the vertical axes represent absorption intensity and emission intensity. As results shown in FIG. 26, the toluene solution with 2,8mmtBuPCA2Bdfpy exhibited absorption peaks at around 383 nm, 336 nm, and 284 nm and an emission peak at around 429 nm (excitation wavelength: 380 nm).

For the measurement of the absorption spectrum of 2,8mmtBuPCA2Bdfpy in the solid thin film state, a spectrophotometer (U4100 Spectrophotometer, manufactured by Hitachi High-Technologies Corporation) was used. The solid thin film was formed over a quartz substrate by a vacuum evaporation method. The emission spectrum of the solid thin film was measured with a fluorescence spectrophotometer (FP-8600, produced by JASCO Corporation).

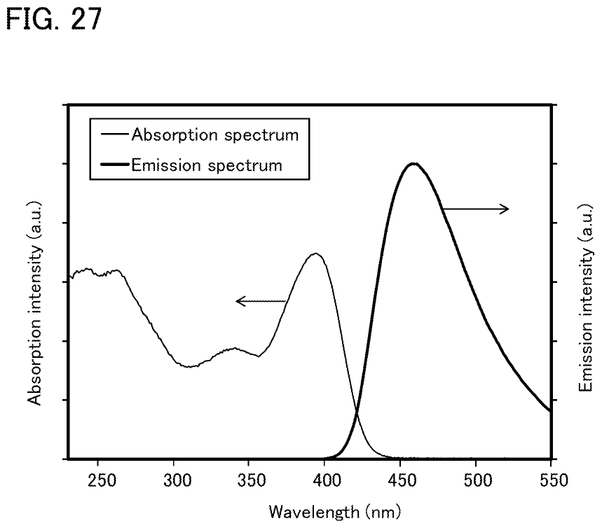

FIG. 27 shows the obtained measurement results of the absorption spectrum and the emission spectrum of 2,8mmtBuPCA2Bdfpy in the solid thin film state. The horizontal axis represents wavelengths and the vertical axes represent absorption intensity and emission intensity. As results shown in FIG. 27, the solid thin film of 2,8tBuCz2Bdfpy exhibited absorption peaks at around 395 nm, 341 nm, 265 nm, and 244 nm and an emission peak at around 459 nm (excitation wavelength: 390 nm).

The results indicate that 2,8mmtBuPCA2Bdfpy emits blue light and can be used as a host for a light-emitting substance or a host for a substance that emits fluorescence in the visible region.

Moreover, the quantum yield in the toluene solution with 2,8mmtBuPCA2Bdfpy was measured. For the measurement of the quantum yield, an absolute PL quantum yield measurement system (Quantaurus-QY, manufactured by Hamamatsu Photonics K.K.) was used.

Furthermore, the measured quantum yield in the toluene solution was as high as 66%, which indicates that 2,8mmtBuPCA2Bdfpy is suitable as a light-emitting material.

### Example 3

### Synthesis Example 3

In this synthesis example, will be described 2,8-bis(3,6-di-tert-butyl-9H-carbazol-9-yl)-benzo[1″,2″: 4,5;5″,4″: 4′,5′]difuro[2,3-b:2′,3′-b′]dipyridine-11-carbonitrile (abbreviation: 11CN-2,8tBuCz2Bdfpy), an organic compound of one embodiment of the present invention, which is represented by Structural Formula (102) in Embodiment 1. A structure of 11CN-2,8tBuCz2Bdfpy is shown below.

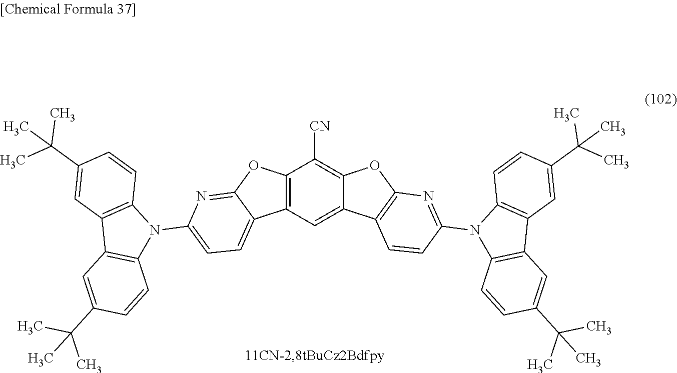 [SMILES: CC(C)(C)c1ccc2c(c1)c1cc(C(C)(C)C)ccc1n2-c1ccc2c(n1)oc1c(C#N)c3oc4nc(-n5c6ccc(C(C)(C)C)cc6c6cc(C(C)(C)C)ccc65)ccc4c3cc12]

### Step 1: Synthesis of 2,6-bis(6-chloro-pyridine-2-yloxy)benzonitrile

Into a 1000 mL three-neck flask, 13 g (96 mmol) of 2,6-difluorobenzonitrile, 25 g (193 mmol) of 6-chloro-2-hydroxypyridine, 53 g (384 mmol) of potassium carbonate, and 500 mL of N,N-dimethylformamide (DMF) were put. The mixture was stirred at 90° C. for 7 hours under a nitrogen stream. After a predetermined time elapsed, the obtained reaction mixture was poured into 200 mL of water, whereby a solid was precipitated. The precipitated solid was subjected to suction filtration to give a white solid. Purification was performed on the obtained solid by high performance liquid chromatography using (mobile phase: chloroform). Thus, 8.3 g of a white solid, which was the target compound, was obtained in a yield of 24%. The obtained white solid was identified as 2,6-bis(6-chloro-pyridine-2-yloxy)benzonitrile by nuclear magnetic resonance (NMR) spectroscopy. The synthesis scheme of Step 1 is shown in Formula (c-1) below.

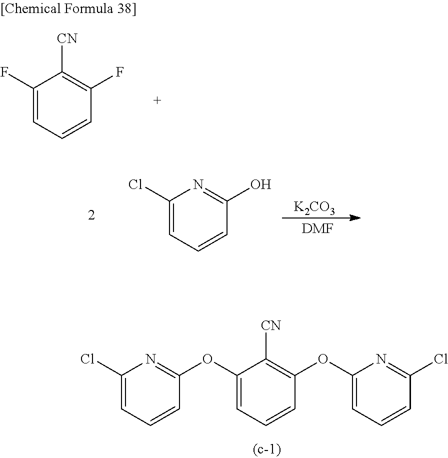

### Step 2: Synthesis of 2,8-dichloro-benzo[1″,2″:4,5;5″,4″:4′,5′]difuro[2,3-b:2′,3′-b]dipyridine-11-carbonitrile

Into a 300 mL three-neck flask, 8.3 g (23 mmol) of 2,6-bis(6-chloro-pyridine-2-yloxy)benzonitrile synthesized in Step 1, 50 g of pivalic acid, 1.7 g (4.6 mmol) of palladium trifluoroacetate, and 19 g (115 mmol) of silver acetate were put. The mixture was stirred at 150° C. for 23 hours in air. After a predetermined time elapsed, the reaction mixture was added with ethyl acetate and suction-filtrated to give a solid. The obtained solid was washed with 2000 mL of heated toluene to give 18 g of a crude product. The obtained crude product was identified as 2,8-dichloro-benzo[1″,2″: 4,5;5″,4″:4′,5′]difuro[2,3-b:2′,3′-b]dipyridine-11-carbonitrile by nuclear magnetic resonance (NMR) spectroscopy. The synthesis scheme of Step 2 is shown in Formula (c-2) below.

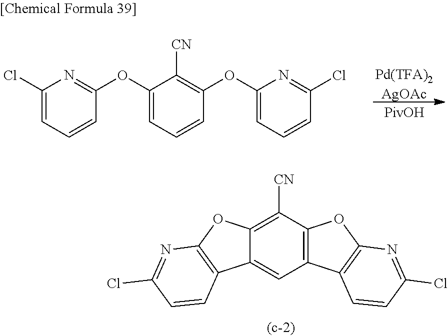

### Step 3: Synthesis of 11CN-2,8tBuCz2Bdfpy

Into a 500 mL of three-neck flask, 18 g of 2,8-dichloro-benzo[1″,2″:4,5;5″,4″:4′,5′]dipyridine-11-carbonitrile (crude product) synthesized in Step 2, 3.5 g (12 mmol) of 3,6-di-tert-butlycarbazole, 2.4 g (25 mmol) of sodium tert-butoxide, and 90 mL of mesitylene were put, and the air in the flask was replaced with nitrogen. After that, the mixture was degassed by being stirred while the pressure in the flask was reduced. After the degassing, the air in the flask was replaced with nitrogen, and the mixture was added with 41 mg of allylpalladium(II)chloride dimer and 80 mg of di-tert-butyl(1-methyl-2,2-diphenylcyclopropyl)phosphine (cBRIDP) and stirred while being heated at 140° C. for 7 hours. After a predetermined time elapsed, the reaction mixture was suction-filtered. The obtained filtrate was concentrated to give a solid. The obtained solid was purified by silica column chromatography. A developing solvent of hexane:toluene=1:2 was used. The obtained fraction was concentrated to give a solid. The obtained solid was recrystallized with a mixed solvent of toluene and ethanol, whereby 0.27 g of a yellow solid was obtained. By a train sublimation method, 0.27 g of the obtained solid was sublimated and purified. The sublimation purification was performed by heating at 395° C. for 20 hours under a pressure of 1.2×10⁻³ Pa. After the sublimation purification, 0.18 g of a yellow solid was obtained at a collection rate of 68%. The synthesis scheme of Step 3 is shown in Formula (c-3) below.

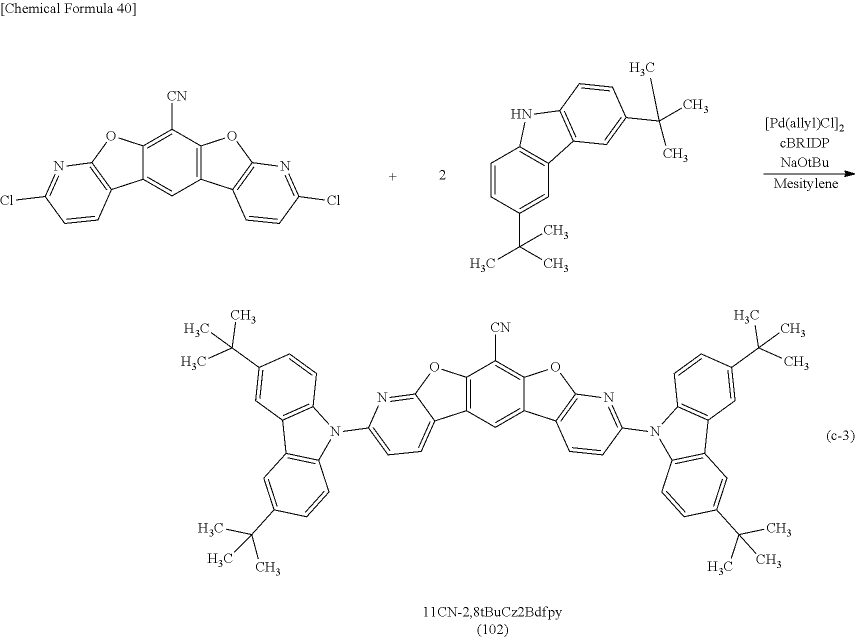 [SMILES: N#Cc1c2oc3nc(Cl)ccc3c2cc2c1oc1nc(Cl)ccc12 ; CC(C)(C)c1ccc2[nH]c3ccc(C(C)(C)C)cc3c2c1 ; CC(C)(C)c1ccc2c(c1)c1cc(C(C)(C)C)ccc1n2-c1ccc2c(n1)oc1c(C#N)c3oc4nc(-n5c6ccc(C(C)(C)C)cc6c6cc(C(C)(C)C)ccc65)ccc4c3cc12]

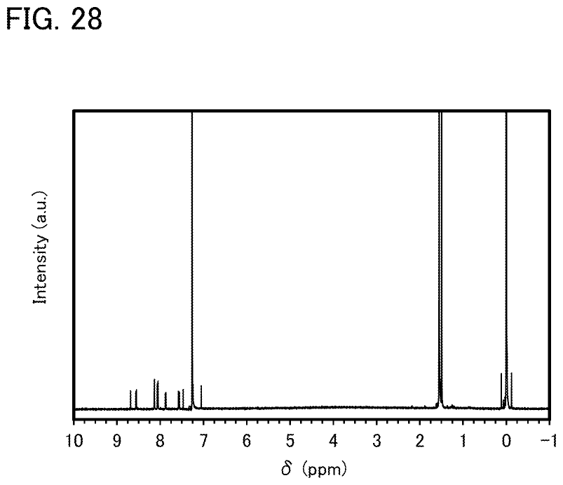

Protons (¹H) of the yellow solid obtained above were measured by nuclear magnetic resonance (NMR) spectroscopy. The obtained values are shown below. FIG. 28 shows a ¹H-NMR chart. The results revealed that 11CN-2,8tBuCz2Bdfpy represented by Structural Formula above was obtained in Synthesis example 3.

¹H-NMR. δ (CDCl₃): 1.49 (s, 36H), 7.57 (d, 4H), 7.87 (d, 2H), 8.06 (d, 4H), 8.14 (s, 4H), 8.56 (d, 2H), 8.69 (s, 1H).

Next, absorption spectra and emission spectra of 11CN-2,8tBuCz2Bdfpy in a toluene solution and a solid thin film were measured.

The absorption spectrum of 11CN-2,8tBuCz2Bdfpy in the toluene solution was measured using an ultraviolet-visible spectrophotometer (V550, produced by JASCO Corporation). The absorption spectrum in the toluene solution was obtained by subtracting the measured absorption spectrum of only toluene put in a quartz cell from the measured absorption spectrum of the toluene solution with 11CN-2,8tBuCz2Bdfpy put in a quartz cell. The emission spectrum in the toluene solution was measured with a fluorescence spectrophotometer (FP-8600, produced by JASCO Corporation).

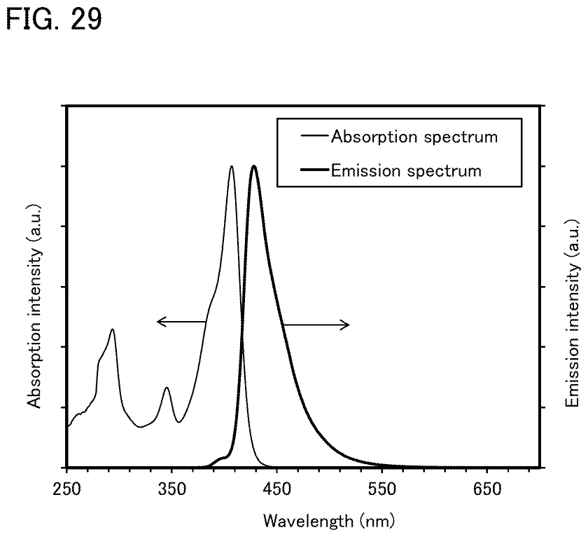

FIG. 29 shows the obtained measurement results of the absorption spectrum and the emission spectrum of 11CN-2,8tBuCz2Bdfpy in the toluene solution. The horizontal axis represents wavelengths and the vertical axes represent absorption intensity and emission intensity.

As results shown in FIG. 29, the toluene solution with 11CN-2,8tBuCz2Bdfpy exhibited absorption peaks at around 407 nm, 347 nm, and 295 nm and an emission peak at around 428 nm (excitation wavelength: 370 nm).

For the measurement of the absorption spectrum of 11CN-2,8tBuCz2Bdfpy in the solid thin film state, a spectrophotometer (U4100 Spectrophotometer, manufactured by Hitachi High-Technologies Corporation) was used. The solid thin film was formed over a quartz substrate by a vacuum evaporation method. The emission spectrum of the solid thin film was measured with a fluorescence spectrophotometer (FP-8600, produced by JASCO Corporation).

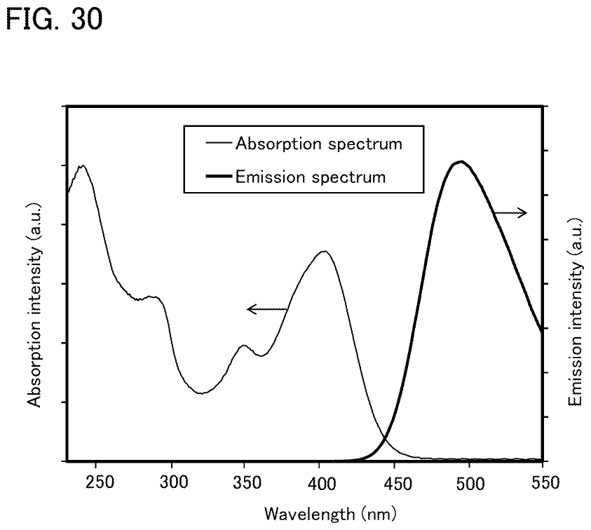

FIG. 30 shows the obtained measurement results of the absorption spectrum and the emission spectrum of 11CN-2,8tBuCz2Bdfpy in the solid thin film state. The horizontal axis represents wavelengths and the vertical axes represent absorption intensity and emission intensity. As results shown in FIG. 30, the solid thin film of 11CN-2,8tBuCz2Bdfpy exhibited absorption peaks at around 404 nm, 350 nm, 290 nm, 240 nm, and 213 nm and an emission peak at around 495 nm (excitation wavelength: 400 nm).

These results indicate that 11CN-2,8tBuCz2Bdfpy emits blue light and can also be used as a host for a light-emitting substance or a host for a substance that emits fluorescence in the visible region.

Furthermore, the measured quantum yield in the toluene solution was as very high as 87%, which indicates that 11CN-2,8tBuCz2Bdfpy is suitable as a light-emitting material. For the measurement of the quantum yield, an absolute PL quantum yield measurement system (Quantaurus-QY, manufactured by Hamamatsu Photonics K.K.) was used.

### Example 4

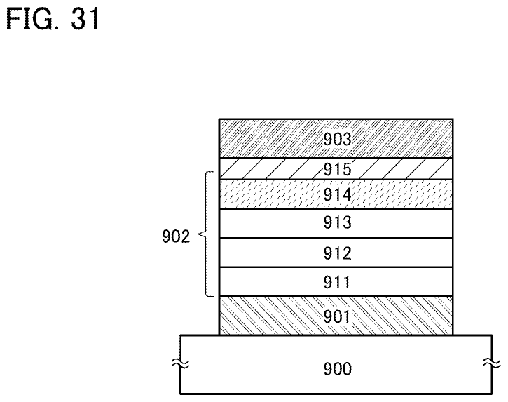

In this example, a light-emitting device 1 using 2,8tBuCz2Bdfpy (Structural Formula (100)) described in Example 1 for a light-emitting layer is regarded as a light-emitting device of one embodiment of the present invention, and an element structure, a manufacturing method, and characteristics of the light-emitting device 1 will be described. Note that FIG. 31 shows an element structure of the light-emitting device used in this example, and Table 1 shows specific components. Chemical formulae of materials used in this example are shown below.

TABLE 1

| | | Hole- | Hole- | | | Electron- | Electron- | |
|---|---|---|---|---|---|---|---|---|
| | First | injection | transport | Light-emitting | | transport | injection | Second |
| | electrode | layer | layer | layer | | layer | layer | electrode |
| Light- | ITSO | DBT3P-II:MoOx | mCzFLP | * | PPT | TmPyPB | LiF | Al |
| emitting | (70 nm) | (1:0.5 30 nm) | (20 nm) | | (5 nm) | (20 nm) | (1 nm) | (200 nm) |
| device 1 | | | | | | | | |

* PPT:2,8tBuCz2Bdfpy (1:0.5, 30 nm)

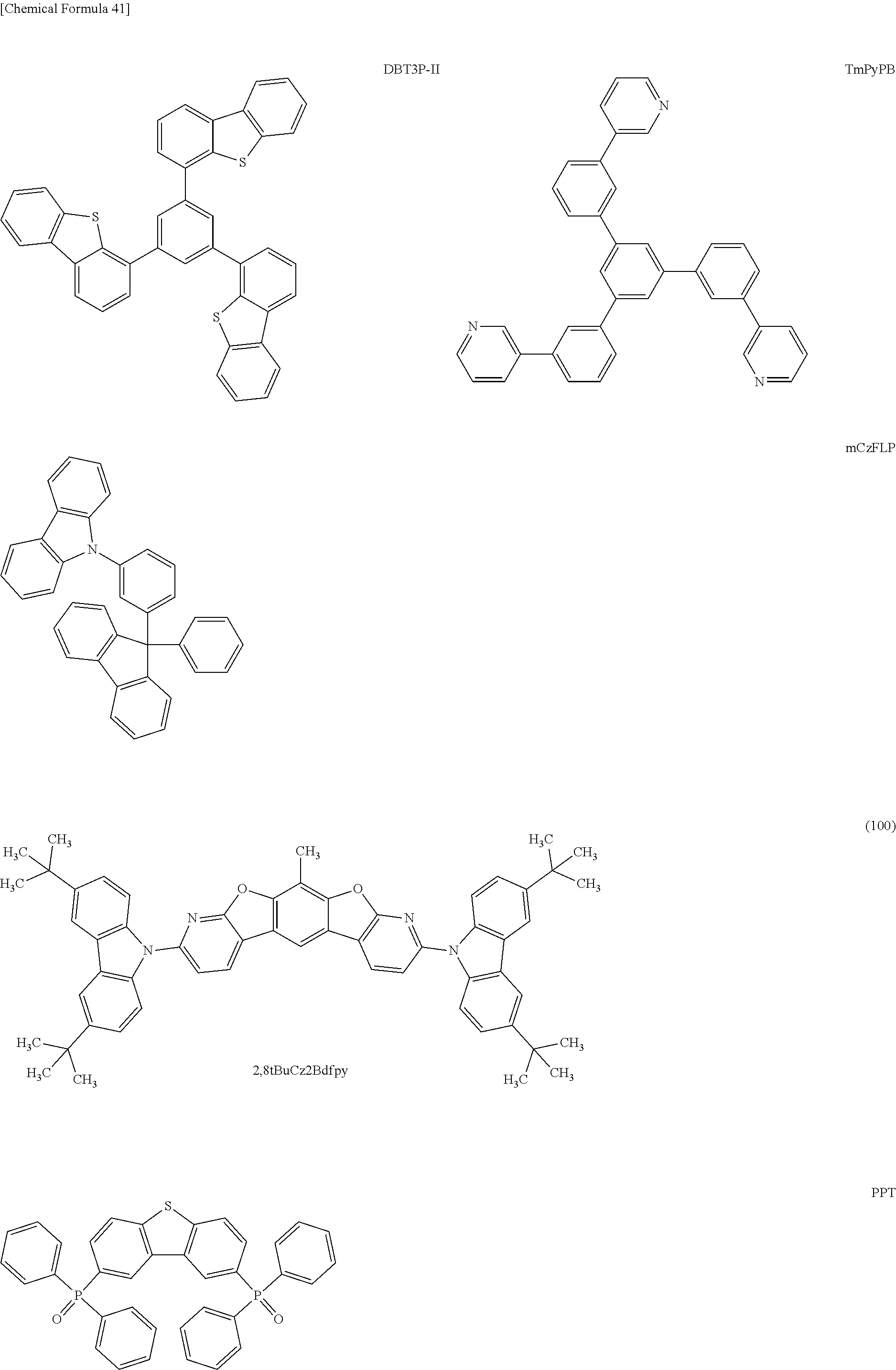 [SMILES: c1ccc2c(c1)sc1c(-c3cc(-c4cccc5c4sc4ccccc45)cc(-c4cccc5c4sc4ccccc45)c3)cccc12 ; c1cncc(-c2cccc(-c3cc(-c4cccc(-c5cccnc5)c4)cc(-c4cccc(-c5cccnc5)c4)c3)c2)c1 ; c1ccc(C2(c3cccc(-n4c5ccccc5c5ccccc54)c3)c3ccccc3-c3ccccc32)cc1 ; Cc1c2oc3nc(-n4c5ccc(C(C)(C)C)cc5c5cc(C(C)(C)C)ccc54)ccc3c2cc2c1oc1nc(-n3c4ccc(C(C)(C)C)cc4c4cc(C(C)(C)C)ccc43)ccc12 ; O=P(c1ccccc1)(c1ccccc1)c1ccc2sc3ccc(P(=O)(c4ccccc4)c4ccccc4)cc3c2c1]

### Fabrication of Light-Emitting Device 1

The light-emitting device 1 shown in this example has a structure, as illustrated in FIG. 31, in which a hole-injection layer 911, a hole-transport layer 912, a light-emitting layer 913, an electron-transport layer 914, and an electron-injection layer 915 are stacked in this order over a first electrode 901 formed over a substrate 900, and a second electrode 903 is stacked over the electron-injection layer 915.

First, the first electrode 901 was formed over the substrate 900. The electrode area was set to 4 mm² (2 mm×2 mm). A glass substrate was used as the substrate 900. The first electrode 901 was formed to a thickness of 70 nm using indium tin oxide containing silicon oxide (ITSO) by a sputtering method.

As pretreatment, a surface of the substrate was washed with water, baking was performed at 200° C. for one hour, and then UV ozone treatment was performed for 370 seconds. After that, the substrate was transferred into a vacuum evaporation apparatus where the inside pressure had been reduced to approximately 10⁻⁴ Pa, and was subjected to vacuum baking at 170° C. for 60 minutes in a heating chamber of the vacuum evaporation apparatus, and then the substrate was cooled down for approximately 30 minutes.

Next, the hole-injection layer 911 was formed over the first electrode 901. For the formation of the hole-injection layer 911, the pressure in the vacuum evaporation apparatus was reduced to 10⁻⁴ Pa, and then 4,4′,4″-(benzene-1,3,5-triyl)tri(dibenzothiophene) (abbreviation: DBT3P-II) and molybdenum oxide were co-evaporated such that DBT3P-II: molybdenum oxide was 1:0.5 (mass ratio) and the obtained thickness was 30 nm.

Then, the hole-transport layer 912 was formed over the hole-injection layer 911. The hole-transport layer 912 was formed to a thickness of 20 nm by evaporation using 9-[3-(9-phenyl-9H-fluoren-9-yl)phenyl]-9H-carbazole (abbreviation: mCzFLP).

Next, the light-emitting layer 913 was formed over the hole-transport layer 912.

For the formation of the light-emitting layer 913, 2,8-bis(diphenylphosphoryl)dibenzo[b,d]thiophene (abbreviation: PPT) and 2,8tBuCz2Bdfpy were co-evaporated such that PPT: 2,8tBuCz2Bdfpy was 1:0.5, and the obtained thickness was 30 nm.

Next, the electron-transport layer 914 was formed over the light-emitting layer 913. The electron-transport layer 914 was formed in such a manner that PET was formed to a thickness of 5 nm by evaporation and then 1,3,5-tri[3-(3-pyridyl)phenyl]benzene (abbreviation: TmPyPB) was formed to a thickness of 20 nm by evaporation.

Then, the electron-injection layer 915 was formed over the electron-transport layer 914. The electron-injection layer 915 was formed by evaporation of lithium fluoride (LiF) to a thickness of 1 nm.

Next, the second electrode 903 was formed over the electron-injection layer 915. The second electrode 903 was formed using aluminum by an evaporation method such that the obtained thickness was 200 nm. In this example, the second electrode 903 functions as a cathode.

Through the above steps, the light-emitting device in which an EL layer was provided between the pair of electrodes over the substrate 900 was formed. The hole-injection layer 911, the hole-transport layer 912, the light-emitting layer 913, the electron-transport layer 914, and the electron-injection layer 915 described in the above steps are functional layers forming the EL layer in one embodiment of the present invention. Furthermore, in all the evaporation steps in the above fabrication method, an evaporation method by a resistance-heating method was used.

The light-emitting device fabricated as described above was sealed using a different substrate (not illustrated). In the case of sealing the light-emitting device using a different substrate (not illustrated), in a glove box containing a nitrogen atmosphere, a sealant was applied so as to surround the light-emitting device formed over the substrate 900, the substrate (not illustrated) provided with a desiccant was made to overlap with a desired position over the substrate 900, and then irradiation with 365 nm ultraviolet light at 6 J/cm 2 was performed.

### Operating Characteristics of Light-Emitting Device 1

Operating characteristics of the fabricated light-emitting device 1 were measured. Note that the measurement was conducted at room temperature (in an atmosphere maintained at 25° C.). Table 2 below shows initial values of the main characteristics of the light-emitting device 1 at around 1000 cd/m².

TABLE 2

| | | | | | | | | External |
|---|---|---|---|---|---|---|---|---|
| | | | Current | | | Current | Power | quantum |
| | Voltage | Current | density | Chromaticity | Luminance | efficiency | efficiency | efficiency |
| | (V) | (mA) | (mA/cm²) | (x, y) | (cd/m²) | (cd/A) | (lm/W) | (%) |
| Light- | 4.4 | 0.23 | 5.6 | (0.156, | 96 | 1.7 | 1.2 | 4.2 |
| emitting | | | | 0.049) | | | | |
| device 1 | | | | | | | | |

The results shown in Table 2 reveals that the light-emitting device 1 of one embodiment of the present invention has favorable operating characteristics such as current-voltage characteristics, power efficiency, and emission efficiency.

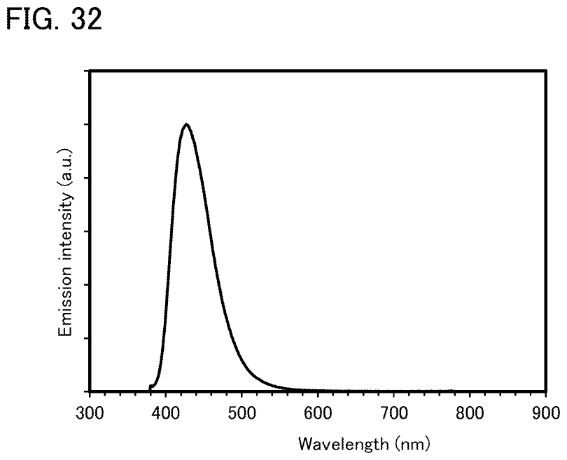

FIG. 32 shows an electroluminescence spectrum of the light-emitting device 1 to which current flows at a current density of 2.5 mA/cm². As shown in FIG. 32, the electroluminescence spectrum of the light-emitting device 1 has a peak at around 427 nm, suggesting that the peak is derived from light emission of 2,8tBuCz2Bdfpy contained in the light-emitting layer 913.

### Example 5

In this example, a light-emitting device 2 containing bis{N-9-(3,5-di-tert-butylphenyl)-9H-carbazol-2-yl]-N-phenyl}-11-methyl-benzo[1″,2″:4,5;5″,4″:4′,5′]difuro[2,3-b:2′,3′-b′]dipyridine-2,8-diamine (abbreviation: 2,8mmtBuPCA2Bdfpy) described in Example 2 and a host material, 3,5-bis[3-(9H-carbazol-9-yl)phenyl]pyridine (abbreviation: 35DCzPPy), in a light-emitting layer is regarded as a light-emitting device of one embodiment of the present invention, and an element structure and characteristics of the light-emitting device 2 will be described. Table 3 shows specific components of the light-emitting device 2 used in this example. Chemical formulae of materials used in this example are shown below.

TABLE 3

| | | Hole- | Hole- | Light- | | | Electron- | |
|---|---|---|---|---|---|---|---|---|
| | First | injection | transport | emitting | Electron-transport | | injection | Second |
| | electrode | layer | layer | layer | layer | | layer | electrode |
| Light- | ITSO | DBT3P-II:MoOx | mCzFLP | * | 35DCzPPy | TmPyPB | LiF | Al |
| emitting | (70 nm) | (1:0.5 30 nm) | (20 nm) | | (10 nm) | (15 nm) | (1 nm) | (200 nm) |
| device 2 | | | | | | | | |

* 35DCzPPy:2,8mmtBuPCA2Bdfpy (0.97:0.03, 30 nm)

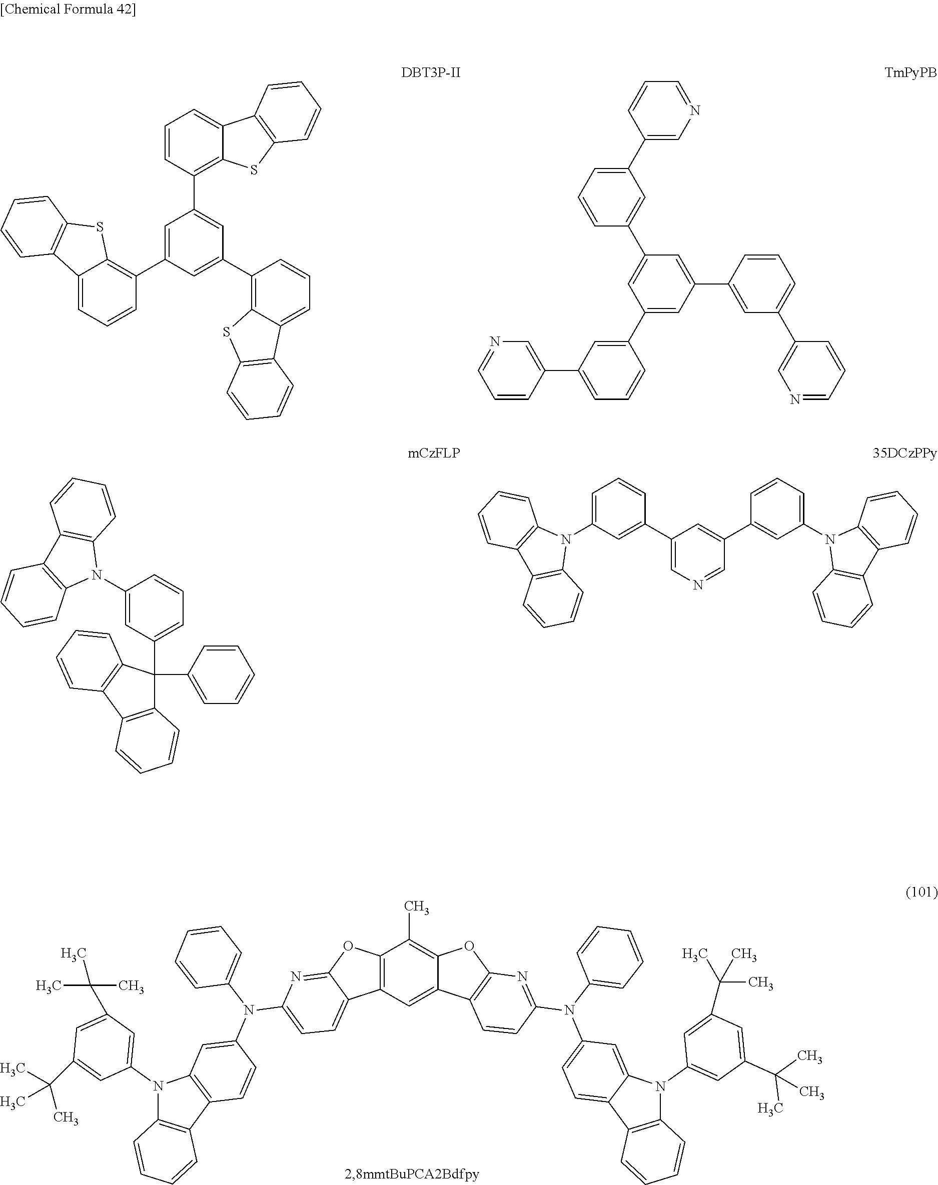 [SMILES: c1ccc2c(c1)sc1c(-c3cc(-c4cccc5c4sc4ccccc45)cc(-c4cccc5c4sc4ccccc45)c3)cccc12 ; c1cncc(-c2cccc(-c3cc(-c4cccc(-c5cccnc5)c4)cc(-c4cccc(-c5cccnc5)c4)c3)c2)c1 ; c1ccc(C2(c3cccc(-n4c5ccccc5c5ccccc54)c3)c3ccccc3-c3ccccc32)cc1 ; c1cc(-c2cncc(-c3cccc(-n4c5ccccc5c5ccccc54)c3)c2)cc(-n2c3ccccc3c3ccccc32)c1 ; Cc1c2oc3nc(N(c4ccccc4)c4ccc5c6ccccc6n(-c6cc(C(C)(C)C)cc(C(C)(C)C)c6)c5c4)ccc3c2cc2c1oc1nc(N(c3ccccc3)c3ccc4c5ccccc5n(-c5cc(C(C)(C)C)cc(C(C)(C)C)c5)c4c3)ccc12]

### <<Fabrication of Light-Emitting Device 2>>

The light-emitting device 2 described in this example has a structure similar to that of the light-emitting device described in Example 4 with use of FIG. 31, in which the hole-injection layer 911, the hole-transport layer 912, the light-emitting layer 913, the electron-transport layer 914, and the electron-injection layer 915 are stacked in this order over the first electrode 901 formed over the substrate 900, and the second electrode 903 is stacked over the electron-injection layer 915.

For the formation of the hole-injection layer 911, 4,4′,4″-(benzene-1,3,5-triyl)tri(dibenzothiophene) (abbreviation: DBT3P-II) and molybdenum oxide (abbreviation: MoOx) were co-evaporated to have a weight ratio of 1:0.5 (=DBT3P-II: MoOx) and a thickness of 30 nm. The hole-transport layer 912 was formed to a thickness of 20 nm by evaporation using 9-[3-(9-phenyl-9H-fluoren-9-yl)phenyl]-9H-carbazole (abbreviation: mCzFLP).

For the formation of the light-emitting layer 913, 35DCzPPy and 2,8mmtBuPCA2Bdfpy were co-evaporated to have a weight ratio of 0.97:0.03 (=35DCzPPy: 2,8mmtBuPCA2Bdfpy) and a thickness of 30 nm.

For the formation of the electron-transport layer 914, 3,5-bis[3-(9H-carbazol-9-yl)phenyl]pyridine (abbreviation: 35DCzPPy) was evaporated to a thickness 10 nm, and then 1,3,5-tri[3-(3-pyridyl)phenyl]benzene (abbreviation: TmPyPB) was evaporated to a thickness of 15 nm.

### <<Operating Characteristics of Light-Emitting Device 2>>

Operating characteristics of the fabricated light-emitting device 2 were measured. Note that the measurement was conducted at room temperature (in an atmosphere maintained at 25° C.). The initial values of main characteristics of the light-emitting device 2 at approximately 1000 cd/m² are listed in Table 4 below.

TABLE 4

| | | | | | | | | External |
|---|---|---|---|---|---|---|---|---|
| | | | Current | | | Current | Power | quantum |
| | Voltage | Current | density | Chromaticity | Luminance | efficiency | efficiency | efficiency |
| | (V) | (mA) | (mA/cm²) | (x, y) | (cd/m²) | (cd/A) | (lm/W) | (%) |
| Light- | 4.0 | 0.20 | 4.9 | (0.152, | 101 | 2.1 | 1.6 | 5.1 |
| emitting | | | | 0.0449) | | | | |
| device 2 | | | | | | | | |

The results shown in Table 4 reveal that the light-emitting device 2 of one embodiment of the present invention has favorable operating characteristics such as current-voltage characteristics, power efficiency, and emission efficiency.

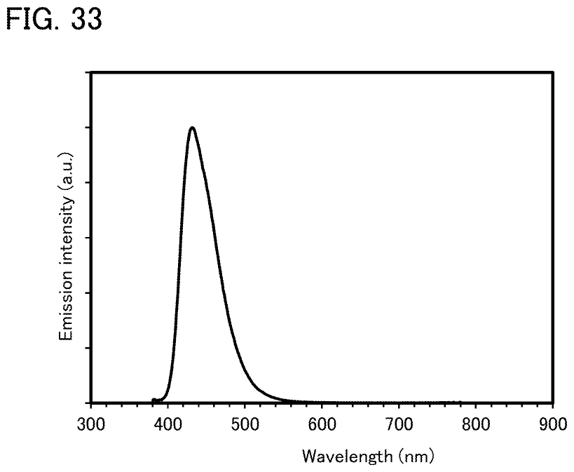

FIG. 33 shows an electroluminescence spectrum of the light-emitting device 2 to which current flows at a current density of 2.5 mA/cm 2. As shown in FIG. 33, the electroluminescence spectrum of the light-emitting device 2 has a peak at around 430 nm, suggesting that the peak is derived from light emission of 2,8mmtBuPCA2Bdfpy contained in the light-emitting layer 913.

### Example 6

In this example, a light-emitting device 3 containing 11CN-2,8tBuCz2Bdfpy described in Example 3 and a host material, 9,9′-(pyrimidine-4,6-diyldi-3,1-phenylene)bis(9H-carbazole) (abbreviation: 4,6mCzP2Pm), in a light-emitting layer is regarded as a light-emitting device of one embodiment of the present invention, and an element structure and characteristics of the light-emitting device 3 will be described. Table 5 shows specific components of the light-emitting device used in this example. Chemical formulae of materials used in this example are shown below.

TABLE 5

| | | Hole- | Hole- | Light- | | | Electron- | |
|---|---|---|---|---|---|---|---|---|
| | First | injection | transport | emitting | Electron-transport | | injection | Second |
| | electrode | layer | layer | layer | layer | | layer | electrode |
| Light- | ITSO | DBT3P-II:MoOx | mCzFLP | * | 35DCzPPy | TmPyPB | LiF | Al |
| emitting | (70 nm) | (1:0.5 30 nm) | (20 nm) | | (10 nm) | (15 nm) | (1 nm) | (200 nm) |
| device 3 | | | | | | | | |

* 4,6mCzP2Pm:11CN-2,8tBuCz2Bdfpy (0.9:0.1, 30 nm)

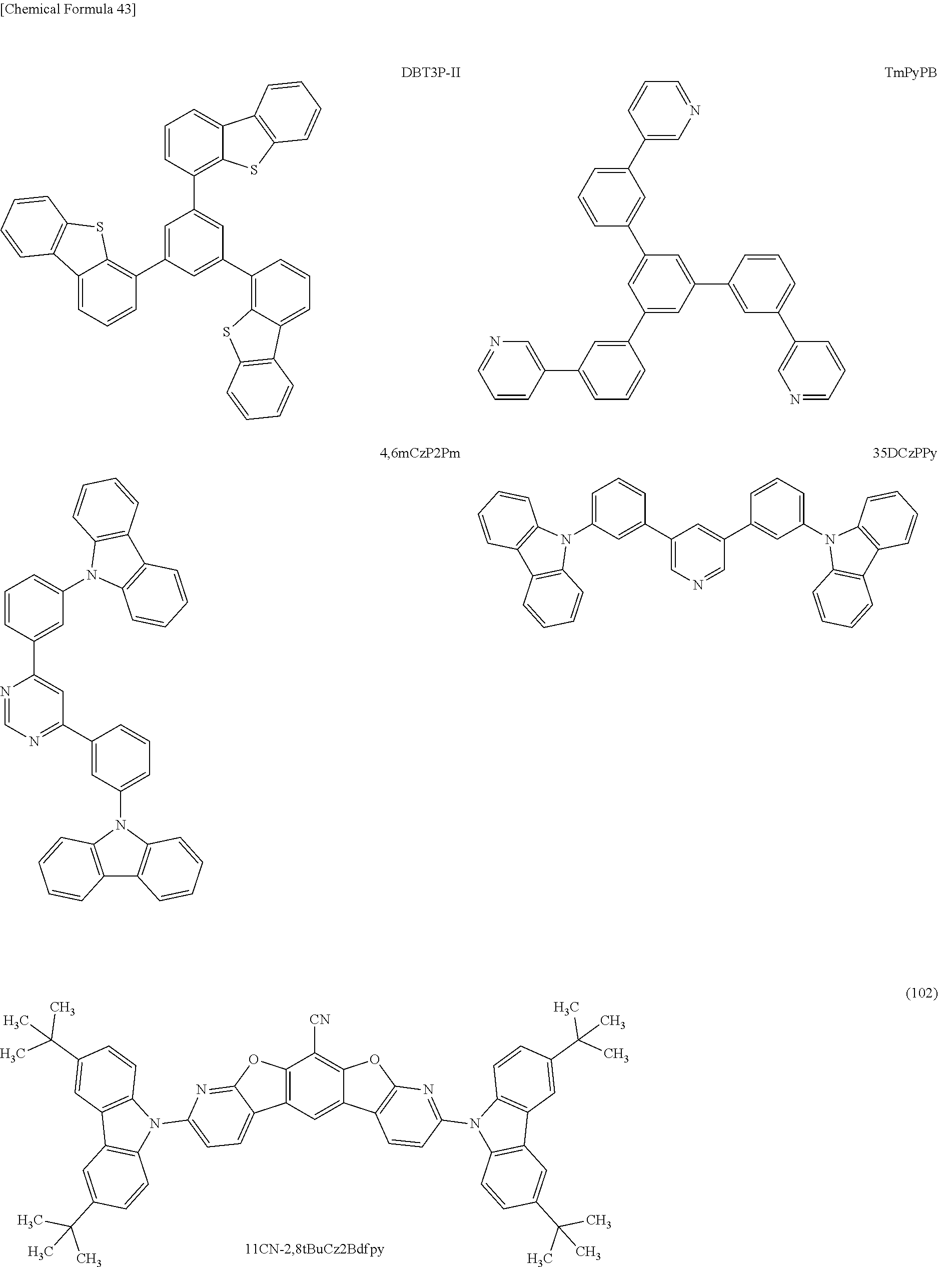 [SMILES: c1ccc2c(c1)sc1c(-c3cc(-c4cccc5c4sc4ccccc45)cc(-c4cccc5c4sc4ccccc45)c3)cccc12 ; c1cncc(-c2cccc(-c3cc(-c4cccc(-c5cccnc5)c4)cc(-c4cccc(-c5cccnc5)c4)c3)c2)c1 ; c1cc(-c2cc(-c3cccc(-n4c5ccccc5c5ccccc54)c3)ncn2)cc(-n2c3ccccc3c3ccccc32)c1 ; c1cc(-c2cncc(-c3cccc(-n4c5ccccc5c5ccccc54)c3)c2)cc(-n2c3ccccc3c3ccccc32)c1 ; CC(C)(C)c1ccc2c(c1)c1cc(C(C)(C)C)ccc1n2-c1ccc2c(n1)oc1c(C#N)c3oc4nc(-n5c6ccc(C(C)(C)C)cc6c6cc(C(C)(C)C)ccc65)ccc4c3cc12]

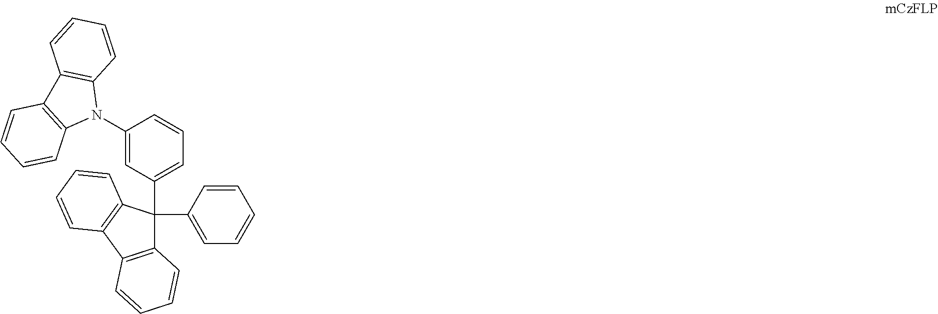 [SMILES: c1ccc(C2(c3cccc(-n4c5ccccc5c5ccccc54)c3)c3ccccc3-c3ccccc32)cc1]

### <<Fabrication of Light-Emitting Device 3>>

The light-emitting device 3 described in this example has a structure similar to that of the light-emitting device described in Example 4 with use of FIG. 31, in which the hole-injection layer 911, the hole-transport layer 912, the light-emitting layer 913, the electron-transport layer 914, and the electron-injection layer 915 are stacked in this order over the first electrode 901 formed over the substrate 900, and the second electrode 903 is stacked over the electron-injection layer 915.

For the formation of the hole-injection layer 911, 4,4′,4″-(benzene-1,3,5-triyl)tri(dibenzothiophene) (abbreviation: DBT3P-II) and molybdenum oxide (abbreviation: MoOx) were co-evaporated to have a weight ratio of 1:0.5 (=DBT3P-II: MoOx) and a thickness of 30 nm. The hole-transport layer 912 was formed to a thickness of 20 nm by evaporation using 9-[3-(9-phenyl-9H-fluoren-9-yl)phenyl]-9H-carbazole (abbreviation: mCzFLP).

For the formation of the light-emitting layer 913, 4,6mCzP2Pm and 11CN-2,8tBuCz2Bdfpy were co-evaporated to have a weight ratio of 0.9:0.1 (=4,6mCzP2Pm: 11CN-2,8tBuCz2Bdfpy) and a thickness of 30 nm.

For the formation of the electron-transport layer 914, 3,5-bis[3-(9H-carbazol-9-yl)phenyl]pyridine (abbreviation: 35DCzPPy) was evaporated to a thickness of 10 nm, and then 1,3,5-tri[3-(3-pyridyl)phenyl]benzene (abbreviation: TmPyPB) was evaporated to have a thickness of 15 nm.

### <<Operating Characteristics of Light-Emitting Device 3>>

Operating characteristics of the fabricated light-emitting device 3 were measured. Note that the measurement was conducted at room temperature (in an atmosphere maintained at 25° C.). The initial values of main characteristics of the light-emitting device 3 at approximately 1000 cd/m² are listed in Table 5 below.

TABLE 6

| | | | | | | | | External |
|---|---|---|---|---|---|---|---|---|
| | | | Current | | | Current | Power | quantum |
| | Voltage | Current | density | Chromaticity | Luminance | efficiency | efficiency | efficiency |
| | (V) | (mA) | (mA/cm²) | (x, y) | (cd/m²) | (cd/A) | (lm/W) | (%) |
| Light- | 3.6 | 0.041 | 1.02 | (0.139, | 100 | 10 | 9 | 7.0 |
| emitting | | | | 0.185) | | | | |
| device 3 | | | | | | | | |

The results shown in Table 5 reveals that the light-emitting device 3 of one embodiment of the present invention has favorable operating characteristics such as current-voltage characteristics, power efficiency, and emission efficiency.

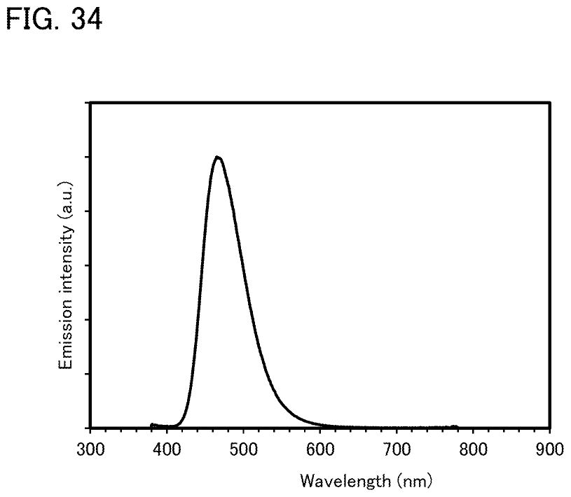

FIG. 34 shows an electroluminescence spectrum of the light-emitting device 3 to which current flows at a current density of 2.5 mA/cm². As shown in FIG. 34, the electroluminescence spectrum of the light-emitting device 3 has a peak at around 465 nm, suggesting that the peak is derived from light emission of 11CN-2,8tBuCz2Bdfpy contained in the light-emitting layer 913.

### REFERENCE NUMERALS

101: first electrode, 102: second electrode, 103, 103a, 103b, 103c: EL layer, 103B, 103G, 103R: EL layer, 103P, 103Q: EL layer, 104, 104a, 104b: hole-injection/transport layer, 104B, 104G, 104R: hole-injection/transport layer, 104P, 104Q: hole-injection/transport layer, 106, 106B, 106G, 106R: charge-generation layer, 107, 107B, 107G, 107R: insulating layer, 108, 108B, 108G, 108R, 108Q: electron-transport layer, 109: electron-injection layer, 111, 111a, 111b: hole-injection layer, 112, 112a, 112b: hole-transport layer, 113, 113a, 113b, 113c: light-emitting layer, 114, 114b: electron-transport layer, 115, 115b: electron-injection layer, 231: display region, 400: substrate, 401: first electrode, 403: EL layer, 404: second electrode, 405, 406: sealant, 407: sealing substrate, 412: pad, 420: IC chip, 501C: insulating film, 501D: insulating film, 504: conductive film, 506: insulating film, 508: semiconductor film, 508A: region, 508B: region, 508C: region, 510: first substrate, 512A: conductive film, 512B: conductive film, 519: terminal, 520: functional layer, 524: conductive film, 528: partition, 528B: opening, 528G: opening, 528R: opening, 530B: pixel circuit, 530G: pixel circuit, 540: insulating layer, 550B: light-emitting device, 550G: light-emitting device, 550R: light-emitting device, 551B: electrode, 551G: electrode, 551R: electrode, 552: electrode, 573: insulating layer, 580: space, 700: light-emitting apparatus, 702B: pixel, 702G: pixel, 702R: pixel, 703: pixel, 705: insulating layer, 770: substrate, 951: substrate, 952: electrode, 953: insulating layer, 954: partition layer, 955: EL layer, 956: electrode, 5200B: electronic device, 5210: arithmetic device, 5220: input/output device, 5230: display portion, 5240: input portion, 5250: sensing portion, 5290: communication portion, 8001: ceiling light, 8002: foot light, 8003: sheet-like lighting, 8004: lighting device, 8005: desk lamp, 8006: light source

1. An organic compound represented by General Formula (G1), 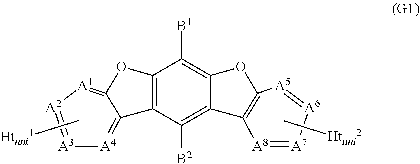
wherein at least one or two of A¹ to A⁴ represent nitrogen, and the others represent carbon,
wherein at least one or two of A⁵ to A⁸ represent nitrogen, and the others represent carbon,
wherein B¹ and B² each independently represent hydrogen, an alkyl group having 1 to 6 carbon atoms, or a cyano group, and
wherein Htᵤₙᵢ¹ and Htᵤₙᵢ² each independently represent a hole-transport skeleton.

2. The organic compound according to claim 1,
wherein the Htᵤₙᵢ¹ and the Htᵤₙᵢ² each independently has a carbazolyl group or an amino group.

3. The organic compound according to claim 1,
wherein the Htᵤₙᵢ¹ and the Htᵤₙᵢ¹ are each independently represented by General Formula (Ht-1) or (Ht-2) below, 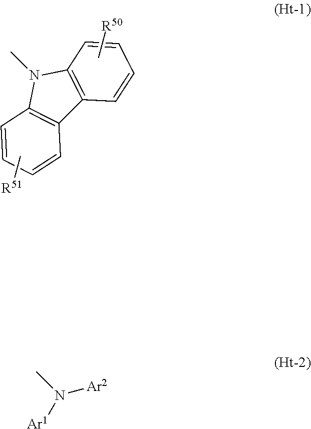
wherein in the formula, R⁵⁰ and R⁵¹ each represent 1 to 4 substituents and independently represent any one of hydrogen, an alkyl group having 1 to 6 carbon atoms, and a substituted or unsubstituted phenyl group, and
wherein Ar¹ and Ar² represent any one of a substituted or unsubstituted phenyl group, a substituted or unsubstituted naphthyl group, a substituted or unsubstituted carbazolyl group, a substituted or unsubstituted fluorenyl group, a substituted or unsubstituted dibenzofuranyl group, and a substituted or unsubstituted dibenzothiophenyl group.

4. The organic compound according to claim 1, wherein General Formula (G1) is represented by General Formula (G2), 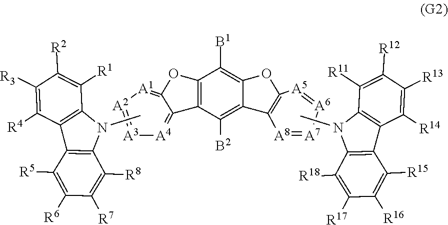
wherein R¹ to R⁸ and R¹¹ to R¹⁸ each independently represent hydrogen, an alkyl group having 1 to 6 carbon atoms, a substituted or unsubstituted monocyclic saturated hydrocarbon group having 5 to 7 carbon atoms, a substituted or unsubstituted polycyclic saturated hydrocarbon group having 7 to 10 carbon atoms, a substituted or unsubstituted aryl group having 6 to 13 carbon atoms, or a substituted or unsubstituted heteroaryl group having 3 to 12 carbon atoms.

5. The organic compound according to claim 1, wherein General Formula (G1) is represented by General Formula (G3), 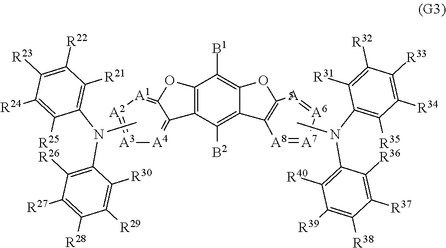
wherein R²¹ to R³⁰ and R³¹ to R⁴⁰ each independently represent hydrogen, an alkyl group having 1 to 6 carbon atoms, a substituted or unsubstituted monocyclic saturated hydrocarbon group having 5 to 7 carbon atoms, a substituted or unsubstituted polycyclic saturated hydrocarbon group having 7 to 10 carbon atoms, a substituted or unsubstituted aryl group having 6 to 13 carbon atoms, or a substituted or unsubstituted heteroaryl group having 3 to 12 carbon atoms.

6. The organic compound according to claim 4, wherein General Formula (G2) is represented by General Formula (G4),

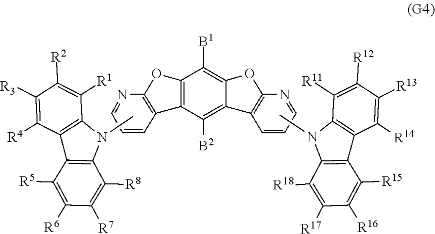

7. The organic compound according to claim 5, wherein General Formula (G3) is represented by General Formula (G5),

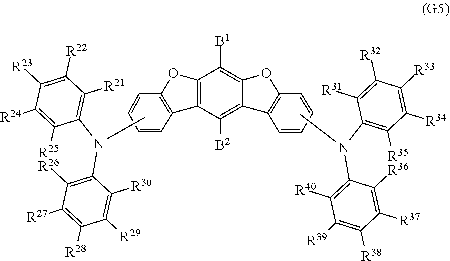

8. An organic compound represented by any one of Structural Formulae (100), (101), and (102),

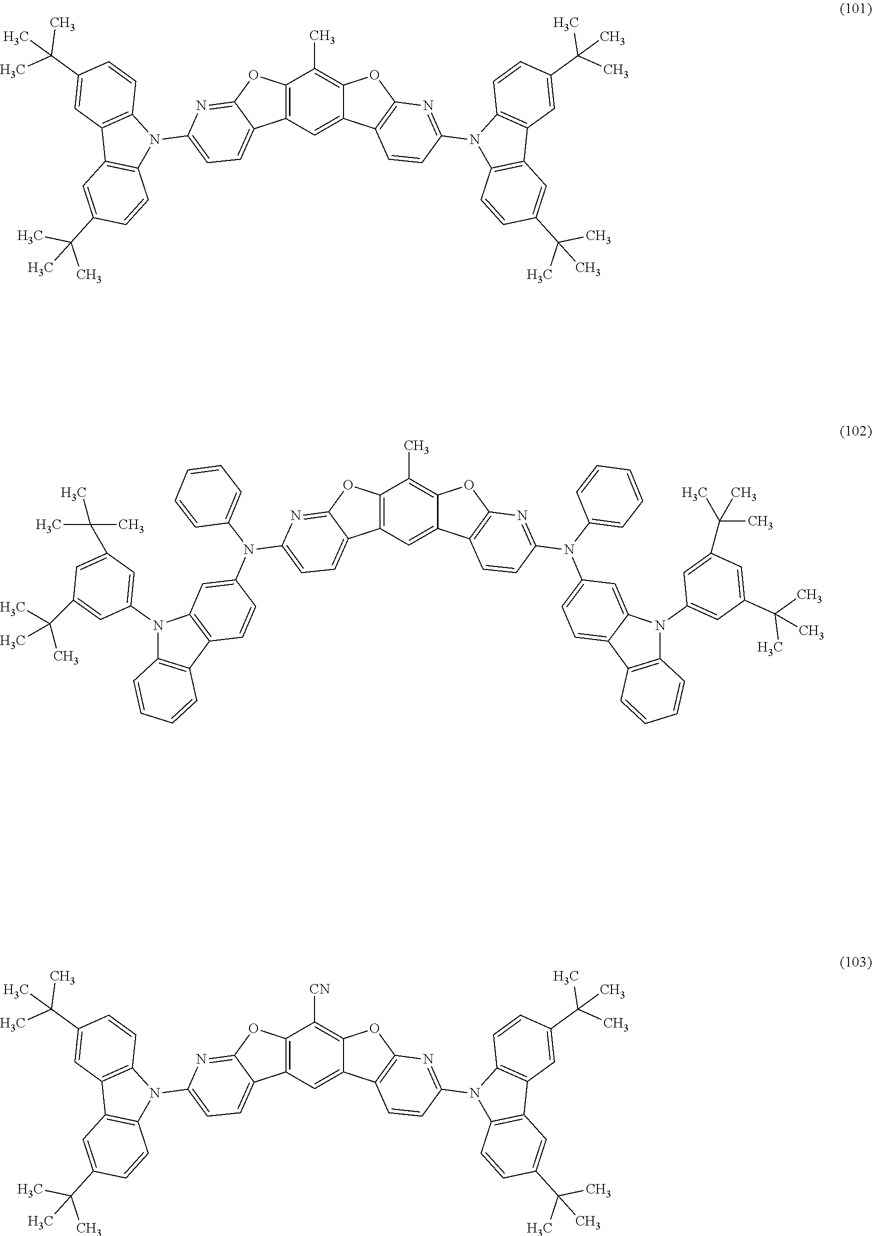

9. A light-emitting device comprising the organic compound according to claim 1.

10. A light-emitting device comprising an EL layer between a pair of electrodes,
wherein the EL layer comprises the organic compound according to claim 1.

11. A light-emitting device comprising an EL layer between a pair of electrodes,
wherein the EL layer comprises a light-emitting layer, and
wherein the light-emitting layer comprises the organic compound according to claim 1.

12. A light-emitting apparatus comprising:
the light-emitting device according to claim 9; and
at least one of a transistor and a substrate.

13. An electronic device comprising:
the light-emitting apparatus according to claim 12; and
at least one of a microphone, a camera, an operation button, an external connection portion, and a speaker.

14. A lighting device comprising:
the light-emitting device according to claim 9; and
at least one of a housing, a cover, and a support.

15. A light-emitting device comprising the organic compound according to claim 8.

16. A light-emitting device comprising an EL layer between a pair of electrodes,
wherein the EL layer comprises the organic compound according to claim 8.

17. A light-emitting device comprising an EL layer between a pair of electrodes,
wherein the EL layer comprises a light-emitting layer, and
wherein the light-emitting layer comprises the organic compound according to claim 8.

18. A light-emitting apparatus comprising:
the light-emitting device according to claim 15; and
at least one of a transistor and a substrate.

19. An electronic device comprising:
the light-emitting apparatus according to claim 18; and
at least one of a microphone, a camera, an operation button, an external connection portion, and a speaker.

20. A lighting device comprising:
the light-emitting device according to claim 15; and
at least one of a housing, a cover, and a support.
